(19) United States
(12) Patent Application Publication
(10) Pub. No.: US 2015/0185368 A1
(43) Pub. Date: Jul. 2, 2015

(54) OPTICAL FILM, POLARIZING PLATE AND LIQUID CRYSTAL DISPLAY USING THE SAME, AND COMPOUND

(71) Applicant: FUJIFILM Corporation, Tokyo (JP)

(72) Inventors: Hisato NAGASE, Kanagawa (JP); Hiroyuki HAGIO, Kanagawa (JP); Hiroyuki ISHIKAWA, Kanagawa (JP); Naoya SHIMOJU, Kanagawa (JP); Aiko YOSHIDA, Kanagawa (JP); Yu NAITO, Kanagawa (JP)

(73) Assignee: FUJIFILM CORPORATION, Tokyo (JP)

(21) Appl. No.: 14/570,499

(22) Filed: Dec. 15, 2014

(30) Foreign Application Priority Data
Dec. 27, 2013 (JP) 2013-273194
Nov. 17, 2014 (JP) 2014-233161

(51) Int. Cl. G02B 1/14; G02B 5/30; G02F 1/1335; C07D 239/62

(52) U.S. Cl. CPC G02B 1/14; C07D 239/62; G02B 5/3025; G02F 1/133528

(57) ABSTRACT

An optical film containing a cellulose acylate, and a compound represented by formula (I); a polarizing plate and a liquid crystal display device using the optical film; and a compound represented by formula (I):

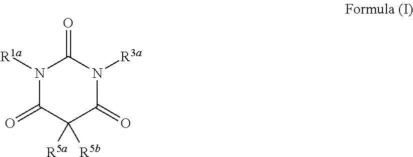

wherein R^{1a} and R^{1a} each independently represent a hydrogen atom, an alkyl group, an alkenyl group, a cycloalkyl group, a cycloalkenyl group, an aryl group, or a heterocyclic group; R^{5a} represents a halogen atom, a cyano group, or a substituent, via a heteroatom or the carbon atom, which has a specific unsaturated moiety in an a-position; R^{5b} represents a substituent; and each group of R^{1a}, R^{3a} and R^{5b} may be substituted with a substituent.

### CROSS-REFERENCE TO RELATED APPLICATION

This non-provisional application claims priority under 35 U.S.C. §119 (a) on Patent Application No. 2013-273194 filed in Japan on Dec. 27, 2013, and Patent Application No. 2014-233161 filed in Japan on Nov. 17, 2014, each of which is entirely herein incorporated by reference.

### TECHNICAL FIELD

The present invention relates to an optical film, a polarizing plate and a liquid crystal display using the same, and a compound.

### BACKGROUND ART

Optical films, such as cellulose acylate films, are used for various liquid crystal displays, as optical elements thereof, such as supports for optical compensation films, and protective films for polarizing plates.

In addition to an indoor use of the liquid crystal display, such as a TV use, a chance of the outdoor use thereof is increased, for example, use as a mobile device. As a result, development of a liquid crystal display is required, which is impervious to the use under the conditions of higher temperature and higher humidity than ever before.

Further, a demand for the liquid crystal display to be impervious to more various uses even under unforgiving conditions is growing, and durability at a higher level than ever before has been required from year to year.

In addition, the liquid crystal display has been increased in size and decreased in thickness mainly in TV applications in recent years, and thus the optical film of a constitutional member is also required to be thinned in accordance therewith. An appropriate hardness and favorable cutting property have been considered to be important for the optical film from the viewpoint of workability as well, and the thinned optical film is further required to be improved in the hardness and cutting property.

In the optical film using a cellulose acylate film, it is known that a specific compound is contained in the film, for further improvement in the performance, or in order to solve various problems in the properties as an optical film or the production thereof

For example, it is proposed a barbituric acid compound having a hydrogen atom at one of the 5-position and having a group with a specific Hammett's σm or σp value at the other of the 5-position (see Patent Literature 1), in order to suppress the fluctuation of the retardation of the optical film due to the environmental humidity. In addition, it is proposed a barbituric acid compound having a hydrogen atom at one of the 5-position and having an aryl group at the other of the 5-position, in order to improve the durability of polarizer (see Patent Literature 2). These are compounds, each of which physicochemically functions as an acid, since both of them have a hydrogen atom at the 5-position. It is also proposed that those are further developed, and that a specific organic acid is contained in the optical film, for the improvement in peeling-off property from a support at the time of the solution film formation and the improvement in durability of the polarizer (see Patent Literature 3).

### CITATION LIST

### Patent Literature

Patent Literature1: JP-A-2011-118135 (“JP-A” means unexamined published Japanese patent application)
Patent Literature2: JP-A-2011-126968
Patent Literature3: JP-A-2012-72348

### Technical Problem

The inventors of the present invention have conducted intensive investigations. As a result, the inventors have found that the compatibility of brittleness with surface hardness is a problem for the thinned cellulose acylate film, in order to maintain the workability equivalent to the film of the related art, which has a sufficient thickness. In the research conducted by the inventors, the inventors have found that some compounds of the barbituric acid derivatives, each of which functions as an acid, exhibit the effects of increasing the hardness of the optical film. However, as a result of further investigations on the durability in various conditions other than the improvement in hardness of the optical film, it is newly demonstrated a problem that the optical film is colored in a specific condition.

In view of such circumstances, the present invention is contemplated for providing: an optical film, which exhibits a hardness and light resistance of the optical film, and which particularly is able to suppress the coloration of the optical film in view of the light resistance; a polarizing plate and a liquid crystal display, each of which maintains the optical properties to be improved in durability, including the display unevenness, by using the same; and a compound which exhibits such performance.

### Solution to Problem

The inventors of the present invention have conducted investigations on various barbituric acid compounds. As a result, the inventors have found that a barbituric acid compound having a specific substituent is effective for solving the problem, whereby the present invention has been completed.

In addition, the inventors have found that the durability of a polarizing plate can be improved, by the use of a cellulose acylate film containing a barbituric acid having the specific structure, as a protective film of the polarizer.

According to the present invention, there is provided the following means:
<1> An optical film, containing a cellulose acylate, and at least one compound represented by formula (I):

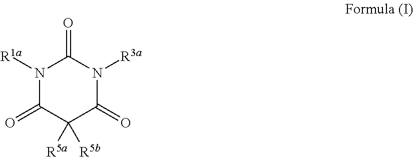

wherein R^{1a} and R^{3a} each independently represent a hydrogen atom, an alkyl group, an alkenyl group, a cycloalkyl group, a cycloalkenyl group, an aryl group, or a heterocyclic group; R^{5a} represents a halogen atom, a cyano group, or a substituent bonded to the barbituric acid skeleton via a heteroatom, —C(═X)—, —C(R^{5a})═Y—, or an ethynyl group; X represents an oxygen atom, a sulfur atom, or N(Ra); Ra represents a hydrogen atom, an alkyl group, an aryl group, a heterocyclic group, a hydroxy group, an acyloxy group, an alkoxy group, an aryloxy group, an alkylthio group, an arylthio group, an amino group, an alkylamino group, an arylamino group, or a heterocyclic amino group; R^{5x} represents a hydrogen atom or a substituent; Y represents a nitrogen atom; R^{5b} represents a substituent; and each group of R^{1a}, R^{3a} and R^{5b} may be substituted with a substituent.
<2> The optical film as described in the item <1>, wherein R^{5a} is a halogen atom, or a substituent bonded to the barbituric acid skeleton via a heteroatom or —C(═X)—.
<3> The optical film as described in the item <1> or <2>, wherein R^{5a} is a substituent bonded to the barbituric acid skeleton via an oxygen atom, a sulfur atom or a nitrogen atom.
<4> The optical film as described in any one of the items <1> to <3>, wherein R^{5a} is a hydroxy group, an alkoxy group, an aryloxy group or an acyloxy group.
<5> The optical film as described in any one of the items <1> to <4>, wherein R^{5a} is a hydroxy group.
<6> The optical film as described in any one of the items <1> to <5>, wherein R^{5b} is an alkyl group, an alkenyl group, a cycloalkyl group, a cycloalkenyl group or an aryl group.
<7> The optical film as described in any one of the items <1> to <6>, wherein a sum of ring structures presented in R^{1a}, R^{3a}, R^{5a} and R^{5b} is 2 or more.
<8> The optical film as described in any one of the items <1> to <7>, wherein at least one of R^{1a} and R^{3a} is an alkyl group, an alkenyl group, a cycloalkyl group, a cycloalkenyl group, an aryl group, or a heterocyclic group.
<9> The optical film as described in any one of the items <1> to <8>, wherein at least one of R^{1a} and R^{3a} is an aryl group, or an alkyl group substituted with an aryl group.
<10> The optical film as described in any one of the items <1> to <9>, further containing a compound represented by formula (A):

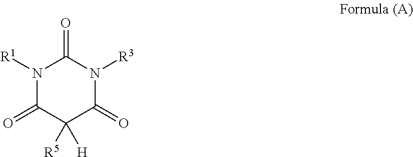

wherein R¹ and R³ each independently represent a hydrogen atom, an alkyl group, an alkenyl group, a cycloalkyl group, a cycloalkenyl group, an aryl group, or a heterocyclic group; R⁵ represents a hydrogen atom or a substituent; and each group of R¹, R³ and R⁵ may be substituted with a substituent.
<11> The optical film as described in the item <10>, wherein a sum of ring structures presented in R¹, R³and R⁵ is 2 or more.
<12> The optical film as described in the item <10> or <11>, wherein R¹, R³ and R⁵ each are a group having a ring structure.
<13> The optical film as described in the item <12>, wherein structures of R¹ and R^{1a} and structures of R³ and R^{3a} are respectively the same as each other, in the compound represented by formula (I) and the compound represented by formula (A) contained in the optical film.
<14> The optical film as described in the item <13>, wherein structures of R⁵ and R^{5b} are further the same as each other, in the compound represented by formula (I) and the compound represented by formula (A) contained in the optical film.
<15> The optical film as described in any one of the items <1> to <14>, wherein the total acyl substitution degree (A) of the cellulose acylate satisfies the following formula.

1.5≦A≦3.0
<16> The optical film as described in any one of the items <1> to <15>, wherein the acyl group of the cellulose acylate is an acetyl group, and the total acetyl substitution degree (B) of the cellulose acylate satisfies the following formula.

2.0≦B≦3.0
<17> A polarizing plate, containing a polarizer, and the optical film as described in any one of the items <1> to <16> provided on at least one face of the polarizer.
<18> A liquid crystal display, at least containing the polarizing plate as described in the item <17>, and a liquid crystal cell.
<19> A compound represented by formula (II):

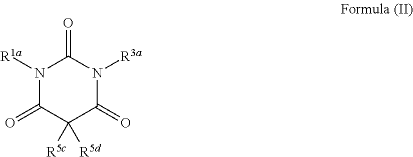

wherein R^{1a} and R^{3a} each independently represent a hydrogen atom, an alkyl group, an alkenyl group, a cycloalkyl group, a cycloalkenyl group, an aryl group, or a heterocyclic group; R^{5c} represents a hydroxy group; R^{5d} represents an alkyl group, an alkenyl group, a cycloalkyl group, a cycloalkenyl group, or an aryl group; each group of R^{1a}, R^{3a} and R^{5d} may be substituted with a substituent; and a sum of ring structures presented in R^{1a}, R^{3a}, and R^{5d} is 2 or more.

Note that, in this specification, any numerical expressions in a style of “. . . to . . . ”will be used to indicate a range including the lower and upper limits represented by the numerals given before and after “to”, respectively.

Herein, in this specification, unless otherwise specified, a group, which is able to have a substituent (for example, a group having an alkyl moiety, an aryl moiety, or a heterocyclic moiety), may have a substituent. For example, the alkyl group is an alkyl group, which may have a substituent, and the aryl group or the aromatic group is an aryl group or an aromatic group, each of which may have a substituent.

In addition, in the case where any atom has at least two substituents and the case where each of the adjacent bonded atoms has a substituent, these substituents may bond to each other to form a ring.

Moreover, in the case where a plurality of groups represented by the same symbol are present and the case where a plurality of groups represented by the same symbol are present as a result of a plurality of repeatings, these may be the same as or different from each other.

When a plurality of substituents, linking groups or the like (hereinafter, referred to as “substituent(s) or the like”) are simultaneously or alternatively defined herein, respective substituents or the like, may be the same as or different from each other.

### ADVANTAGEOUS EFFECTS OF INVENTION

According to the present invention, it is possible to provide: an optical film, which exhibits a hardness and light resistance of the optical film, and in which the coloration of the optical film is suppressed particularly, in view of the light resistance; a polarizing plate and a liquid crystal display, each of which maintain the optical properties and are improved in durability, including the display unevenness, by using the optical film; and a compound, which exhibits such performance.

Other and further objects, features and advantages of the invention will appear more fully from the following description, appropriately referring to the accompanying drawings.

### BRIEF DESCRIPTION OF THE DRAWING

FIG. 1 is an example diagrammatically showing an internal structure of the liquid crystal display of the present invention.

FIG. 2 is an example diagrammatically showing another internal structure of the liquid crystal display of the present invention.

### DETAILED DESCRIPTION OF THE INVENTION

Hereinafter, the present invention is described in detail referring to the embodiments.

### [Optical Film]

The optical film of the present invention is composed of at least one layer of a cellulose acylate film containing a cellulose acylate and at least one compound represented by formula (I). In addition, the cellulose acylate film may be constituted by a plurality of layers, and the compound represented by formula (I) may be contained in any of the layers or may be contained in all of the layers.

Herein, the cellulose acylate film or layer means that the cellulose acylate is contained at 50% by mass or more in the resin component constituting the film or layer, the content of cellulose acylate in the resin component is preferably 60% by mass or more and more preferably 80% by mass or more.

On the other hand, the optical film of the present invention does not contain the cellulose acylate in another layer as a resin component other than the above cellulose acylate film, or the cellulose acylate may form a multilayer configuration together with a layer at less than 50% by mass of the entire resin component even if it is contained. As such a layer, a layer that is specialized in a specific function may be exemplified, and, for example, a hard coat layer may be mentioned.

For example, an anti-glare layer, a clear hard coat layer, an antireflective layer, an antistatic layer, and an antifouling layer may be mentioned, in addition to the hard coat layer. It is a preferred embodiment in the present invention that these layers are provided on the hard coat layer.

The optical film of the present invention is useful in various applications, such as a polarizing plate protective film, and a surface protective film disposed on the image display surface.

### <<Cellulose Acylate Film>>

In the present invention, as described above, the cellulose acylate film is composed of a film in which the proportion of cellulose acylate in the components constituting the resin is 50% by mass or more, and it is an optical film in the narrow sense in the present invention.

The cellulose acylate film may be either a single layer or a layered product having at least two layers, as mentioned above. In the case where the cellulose acylate film is the layered product having at least two layers, a double-layered structure or a three-layered structure is preferable, and a three-layered structure is more preferable. In the case of a three-layered structure, it is preferable to have one layer of a core layer (that is, it is the thickest layer, and it is also referred to as the base layer hereinafter), and a skin layer A and a skin layer B, which sandwich the core layer. That is to say, the cellulose acylate film of the present invention preferably has the three-layered structure formed of: skin layer B/core layer/ skin layer A. The skin layer A is a layer brought into contact with the metal support, which will be described below, and the skin layer B is a layer at the interface with the air on the side opposite to the metal support, when the cellulose acylate film is produced by the solution film formation. It is noted that, generally, both the skin layer A and the skin layer B are also referred to as a skin layer (or a surface layer).

In the present invention, the cellulose acylate film contains a cellulose acylate, and at least one compound represented by formula (I).

### <Compound Represented by Formula (I)>

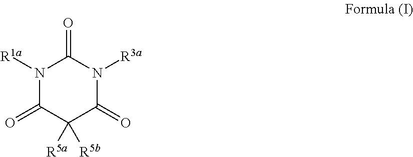

In formula (I), R^{1a} and R^{3a} each independently represent a hydrogen atom, an alkyl group, an alkenyl group, a cycloalkyl group, a cycloalkenyl group, an aryl group, or a heterocyclic group. R^{5a} represents a halogen atom, a cyano group, or a substituent bonded to the barbituric acid skeleton via a heteroatom, —C(═X)—, —C(R^{5x})═Y— or an ethynyl group. Herein, X represents an oxygen atom, a sulfur atom, or N(Ra); Ra represents a hydrogen atom, an alkyl group, an aryl group, a heterocyclic group, a hydroxy group, an acyloxy group, an alkoxy group, an aryloxy group, an alkylthio group, an arylthio group, an amino group, an alkylamino group, an arylamino group, or a heterocyclic amino group; R^{5x} represents a hydrogen atom or a substituent; and Y represents a nitrogen atom. R^{5b} represents a substituent. Each group of R^{1a}, R^{3a} and R^{5b} may be substituted with a substituent.

The mechanism of the compound represented by formula (I) is not clear, but is presumed as follows. The compound represented by formula (I) is a 6-membered ring in the same manner as β-glucose, which is the basic unit of cellulose, in addition to having three carbonyl groups and two nitrogen atoms at the partial structure which constitutes the basic skeleton of barbituric acid, R^{5a} is considered to have a structure in which the atom bonded to the 6-membered ring is a polar atom (for example, an oxygen atom, a sulfur atom, a nitrogen atom, and a halogen atom), a polar carbon to be δ+ by the electronic effect of the heteroatom is present as in a cyano group or an acyl group. Thus, the atom approaches to the hydroxy group, the ether bond, and the position similar thereto of this β-glucose, and is able to interact with each of these polar groups. Then, it is possible for the compound represented by formula (I), more effectively, to interact with the cellulose acylate through the hydrogen bonding and the like and to be present near the main chain of cellulose acylate. As a result, it is considered that the compound represented by formula (I) decreases the free volume in cellulose acylate present between the main chains of cellulose acylate so as to contribute to the improvement or maintenance of the hardness.

In addition, the compound represented by formula (I) does not form any enol form, since a hydrogen atom is not substituted at the 5-position. By having such a structure, the absorption wavelength of the compound represented by formula (I) becomes a short wavelength, and it is possible to suppress the light absorption in the long wavelength ultraviolet region. Thus, it is possible to suppress the coloration of the optical film in an environment in which light reaches, and it is believed that the compound represented by formula (I) contributes to the provision of a liquid crystal display excellent in display performance.

Furthermore, a polarizing plate using a film containing the compound represented by formula (I) as a polarizing plate protective film, can suppress the increase in the perpendicular transmittance over time period at a high temperature and a high humidity. It is believed that this is because the effect of decreasing the water-vapor transmission ratio by decreasing the free volume in cellulose acylate is remarkably great.

The number of carbon atoms of the alkyl group of R^{1a} and R^{3a} in formula (I) is preferably 1 to 20, more preferably 1 to 10, and further preferably 1 to 5.

Examples of the alkyl group include methyl, ethyl, isopropyl, n-butyl, t-butyl, 2-ethylhexyl, n-octyl, n-decyl, n-octadecyl, and isooctadecyl.

The alkyl group may have a substituent. Examples of such a substituent include those exemplified as the following substituent S.

### [Substituent S]

The substituent S include: alkyl groups (preferably those having from 1 to 20 carbon atoms, for example, methyl, ethyl, isopropyl, t-butyl, pentyl, heptyl, 1-ethylpentyl, 2-ethylhexyl, benzyl, 2-ethoxyethyl, 1-carboxymethyl); alkenyl groups (preferably those having from 2 to 20 carbon atoms, for example, vinyl, allyl, oleyl); alkynyl groups (preferably those having from 2 to 20 carbon atoms, for example, ethynyl, butadiynyl, phenylethynyl); cycloalkyl groups (preferably those having from 3 to 20 carbon atoms, for example, cyclopropyl, cyclopentyl, cyclohexyl, 4-methylcyclohexyl); aryl groups (preferably those having from 6 to 20 carbon atoms, for example, phenyl, 1-naphtyl, 4-methoxyphenyl, 2-chlorophenyl, 3-methylphenyl); heterocyclic groups (those preferably having from 0 to 20 carbon atoms and preferably having a ring-constituting heteroatom selected from an oxygen atom, a nitrogen atom or a sulfur atom, and those preferably having a 5-or 6-membered ring which may be condensed with a benzene ring or a hetero ring, and the ring may be a saturated ring, an unsaturated ring or an aromatic ring, for example, 2-pyridyl, 4-pyridyl, 2-imidazolyl, 2-benzoimidazolyl, 2-thiazolyl, 2-oxazolyl); alkoxy groups (preferably those having from 1 to 20 carbon atoms, for example, methoxy, ethoxy, isopropyloxy, benzyloxy); aryloxy groups (preferably those having from 6 to 20 carbon atoms, for example, phenoxy, 1-naphthyloxy, 3-methylphenoxy, 4-methoxyphenoxy); alkylthio groups (preferably those having from 1 to 20 carbon atoms, for example, methylthio, ethylthio, isopropylthio, benzylthio); arylthio groups (preferably those having from 6 to 20 carbon atoms, for example, phenylthio, 1-naphtylthio, 3-methylphenylthio, 4-methoxyphenylthio); acyl groups (those including an alkylcarbonyl group, an alkenylcarbonyl group, an arylcarbonyl group and a heterocyclic carbonyl group, and preferably having 20 or less carbon atoms, for example, acetyl, pivaloyl, acryloyl, methacryloyl, benzoyl, nicotinoyl); alkoxycarbonyl groups (preferably those having from 2 to 20 carbon atoms, for example, ethoxycarbonyl, 2-ethylhexyloxycarbonyl); aryloxycarbonyl groups (preferably those having from 7 to 20 carbon atoms, for example, phenyloxycarbonyl, naphthyloxycarbonyl); amino groups (those including an amino group, an alkylamino group, an arylamino group and a heterocyclic amino group, and preferably having from 0 to 20 carbon atoms, for example, amino, N,N-dimethylamino, N,N-diethylamino, N-ethylamino, anilino, 1-pyrrolidinyl, piperidino, morpholinyl); alkyl- or aryl-sulfonamido groups (preferably those having from 0 to 20 carbon atoms, for example, N,N-dimethylsulfonamido, N-phenylsulfonamido); alkyl- or aryl-sulfamoyl groups (preferably those having from 0 to 20 carbon atoms, for example, N,N-dimethylsulfamoyl, N-phenylsulfamoyl); acyloxy groups (preferably those having from 1 to 20 carbon atoms, for example, acetyloxy, benzoyloxy); alkyl- or aryl-carbamoyl groups (preferably those having from 1 to 20 carbon atoms, for example, N,N-dimethyl carbamoyl, N-phenylcarbamoyl); acylamino groups (preferably those having from 1 to 20 carbon atoms, for example, acetylamino, acryloylamino, benzoylamino, nicotine amido); a cyano group; a hydroxy group; a mercapto group; and a halogen atom (for example, fluorine atom, chlorine atom, bromine atom, iodine atom).

Any of these substituents may be further substituted with a substituent. Examples of such a substituent include those exemplified as the substituent S.

Examples thereof include an aralkyl group in which an alkyl group is substituted with an aryl group, and a group in which an alkyl group is substituted with an alkoxycarbonyl group or a cyano group.

Preferred examples of the substituent for substituting the alkyl group of R^{1a} and R^{3a} include an aryl group, an alkoxycarbonyl group, and a cyano group.

Examples of such a substituted alkyl group include an aralkyl group (preferably a benzyl group), and an alkyl group substituted with an alkoxycarbonyl group or a cyano group at the 2- or 3-position (preferably a 1-alkoxycarbonylmethyl group, 2-(alkoxycarbonyl)ethyl group, or a 2-cyanoethyl group).

The number of carbon atoms of the alkenyl group of R^{1a} and R^{3a} in formula (I) is preferably 2 to 20, more preferably 2 to 10, and further preferably 2 to 5.

Examples of the alkenyl group include vinyl, allyl, isopropenyl, 2-pentenyl, and oleyl.

The alkenyl group may have a substituent. Examples of such a substituent include those exemplified as the substituent S.

The number of carbon atoms of the cycloalkenyl group of R^{1a} and R^{3a} in formula (I) is preferably 5 to 20, more preferably 5 to 10, and further preferably 5 or 6.

Examples of the cycloalkenyl group include cyclopentenyl, and cyclohexenyl.

The cycloalkenyl group may have a substituent. Examples of such a substituent include those exemplified as the substituent S.

The number of carbon atoms of the aryl group of R^{1a} and R^{3a} in formula (I) is preferably 6 to 20, more preferably 6 to 10, and further preferably 6 to 8.

Examples of the aryl group include phenyl, and naphtyl.

The aryl group may have a substituent. Examples of such a substituent include those exemplified as the substituent S.

The number of carbon atoms of the heterocyclic group of R^{1a} and R^{3a} in formula (I) is preferably 0 to 20, more preferably 1 to 10, further preferably 2 to 10, and particularly preferably 2 to 5.

The hetero ring of the heterocyclic group is preferably a 5- or 6-membered hetero ring. The hetero ring may be substituted with a substituent, or may be condensed with a benzene ring, an aliphatic ring, or a hetero ring. Herein, examples of such a substituent include those exemplified as the substituent S.

Examples of the heteroatom for constituting the hetero ring of the heterocyclic group include a nitrogen atom, an oxygen atom, and a sulfur atom. The hetero ring may be an aromatic hetero ring or a non-aromatic hetero ring.

Examples of the hetero ring of the heterocyclic group include a thiophene ring, a furan ring, a pyrrole ring, a pyrazole ring, an imidazole ring, a thiazole ring, an oxazole ring, a triazole ring, a tetrazole ring, a pyridine ring, a pyrazine ring, a triazole ring, a pyrrolidine ring, a pyrroline ring, a pyrazolidine ring, a piperidine ring, a piperazine ring, a morpholine ring, a thiomorpholine ring, and a ring formed by condensing any one of these ring with a benzene ring (e.g. an indole ring, and a benzimidazole ring).

In formula (I), R^{5a} represents a halogen atom, a cyano group, or a substituent bonded to the barbituric acid skeleton via a heteroatom, —C(═X)—, —C(R^{5x})═Y— or an ethynyl group. Herein, X represents an oxygen atom, a sulfur atom, or N(Ra); Ra represents a hydrogen atom, an alkyl group, an aryl group, a heterocyclic group, a hydroxy group, an acyloxy group, an alkoxy group, an aryloxy group, an alkylthio group, an arylthio group, an amino group, an alkylamino group, an arylamino group, or a heterocyclic amino group; R^{5x} represents a hydrogen atom or a substituent; and Y represents a nitrogen atom.

With respect to R^{5a} in formula (I), the substituent bonded to the barbituric acid skeleton via a heteroatom, —C(═X)—, —C(R^{5x})═Y— or an ethynyl group is preferably a group represented by -L-Rz. Herein, L represents a divalent group, and is a heteroatom, —C(═)—, —C(R^{5x})═Y—, an ethynyl group, or a group composed of a combination thereof. Rz represents a hydrogen atom or a substituent. Examples of such a substituent include those exemplified as the substituent S. When Rz is a substituent, it is preferably an alkyl group, an aryl group, or a heterocyclic group; more preferably an alkyl group, or an aryl group.

Rz is preferably a hydrogen atom or the substituent, more preferably a hydrogen atom.

The heteroatom in R^{5a} is preferably an oxygen atom, a sulfur atom, or a nitrogen atom; more preferably an oxygen atom, or a nitrogen atom; and further preferably an oxygen atom.

Examples of the sulfur atom include —S—, —SO—, and —SO₂—. Of these, —S— or —SO₂— is preferable, and —S— is more preferable.

With respect to —C(═)—in R^{5a}, X is preferably an oxygen atom.

With respect to Ra in N(Ra) of R^{5a}, an alkyl group, an aryl group, a heterocyclic group, a hydroxy group, an acyloxy group, an alkoxy group, an aryloxy group, an alkylthio group, an arylthio group, an amino group, an alkylamino group, an arylamino group, or a heterocyclic amino group has the same meaning as the corresponding group in the substituent S, and the preferred range thereof is also the same.

Ra is preferably an alkyl group, an aryl group, a hydroxy group, an acyloxy group, or an alkoxy group.

With respect to —C(R^{5x})═Y— in R^{5a}, R^{5x} represents a hydrogen atom or a substituent. Examples of such a substituent include those exemplified as the substituent S. R^{5x} is preferably a hydrogen atom, an alkyl group, an aryl group, an amino group, an alkylamino group, an arylamino group, or a heterocyclic amino group.

Examples of the substituent bonded to the barbituric acid skeleton via an oxygen atom as represented by R^{5a} specifically include a hydroxy group, an alkoxy group, an aryloxy group, a heterocyclic oxy group, an acyloxy group, an alkyloxycarbonyloxy group, an alkylaminocarbonyloxy group, an arylaminocarbonyloxy group, a heterocyclicaminocarbonyloxy group, and a hydrazinocarbonyloxy group. Of these, it is preferably a hydroxy group, an alkoxy group, an aryloxy group, an acyloxy group, an alkyloxycarbonyloxy group, an alkylaminocarbonyloxy group, or an arylaminocarbonyloxy group; more preferably a hydroxy group, an alkoxy group, an aryloxy group, or an acyloxy group; and further preferably a hydroxy group.

Examples of the substituent bonded to the barbituric acid skeleton via a nitrogen atom as represented by R^{5a} specifically include an amino group, an alkylamino group, an arylamino group, a heterocyclic amino group, and a hydrazino group. Of these, it is preferably an alkylamino group, or an arylamino group.

Examples of the substituent bonded to the barbituric acid skeleton via a sulfur atom as represented by R^{5a} specifically include a mercapto group, an alkylthio group, an arylthio group, an alkyl- or aryl-sulfinyl group, an alkyl- or aryl-sulfonyl group, and an acylthio group. Of these, it is preferably a mercapto group, an alkylthio group, an arylthio group, an alkyl- or aryl-sulfinyl group, or an alkyl- or aryl-sulfonyl group; more preferably an alkylthio group, an arylthio group, or an alkyl- or aryl-sulfonyl group; and further preferably an alkylthio group, or an arylthio group.

Examples of the substituent bonded to the barbituric acid skeleton via —C(═)—as represented by R^{5a} include an acyl group, a thioacyl group, an alkoxycarbonyl group, an aryloxycarbonyl group, a carbamoyl group, and a hydrazinocarbonyl group. Of these, it is preferably an acyl group.

In R^{5a}, examples of the halogen atom include a fluorine atom, a chlorine atom, a bromine atom, and an iodine atom.

Of these, R^{5a} is preferably a halogen atom, or a substituent bonded to the barbituric acid skeleton via a heteroatom or —C(═)—; more preferably a halogen atom, or a substituent bonded to the barbituric acid skeleton via a heteroatom or —C(═O)—; further preferably a halogen atom, or a substituent bonded to the barbituric acid skeleton via a heteroatom; particularly preferably a halogen atom, or a substituent bonded to the barbituric acid skeleton via an oxygen atom or a nitrogen atom; further particularly preferably a halogen atom, or a substituent bonded to the barbituric acid skeleton via an oxygen atom; and most preferably a substituent bonded to the barbituric acid skeleton via an oxygen atom.

In formula (I), R^{5b} represents a substituent. Examples of such a substituent include those exemplified as the substituent S. It is preferably an alkyl group, an alkenyl group, a cycloalkyl group, a cycloalkenyl group, an aryl group, or a heterocyclic group; more preferably an alkyl group, an alkenyl group, a cycloalkyl group, a cycloalkenyl group, or an aryl group; and further preferably an alkyl group, a cycloalkyl group, or an aryl group.

Among the substituent represented by R^{5b}, the above-described alkyl group, alkenyl group, cycloalkyl group, cycloalkenyl group, aryl group and heterocyclic group have the same meaning as the alkyl group, alkenyl group, cycloalkyl group, cycloalkenyl group, aryl group and heterocyclic group exemplified as R^{1a} and R^{3a}, respectively, and preferable ranges are also the same.

Of these, R^{5b} is preferably a substituent having a ring structure.

Herein, the ring may be any one of a saturated carbocyclic ring, an unsaturated carbocyclic ring, an aromatic carbocyclic ring, and a heterocyclic ring, and any of these rings may be directly substituted to the barbituric acid skeleton or may be substituted via a linking group.

These rings are preferably a ring of the group mentioned in the cycloalkyl group, the cycloalkenyl group, the aryl group or the heterocycle of R^{1a} and R^{3a}; and more preferably a cyclohexane ring or a benzene ring.

R^{5b} is preferably a cyclohexyl group, a phenyl group, or a benzyl group; and particularly preferably a phenyl group, or a benzyl group.

The sum of the ring structures presented in R^{1a}, R^{3a}, R^{5a}, and R^{5b} in the compound represented by formula (I) according to the present invention, is preferably 1 or more, and more preferably from 1 to 6. In addition, the sum is still more preferably 2 or more, and particularly preferably from 2 to 6. Among them, the sum is preferably from 2 to 4, and most preferably 2 or 3.

Among these, in the case where the number of ring structures is three, a compound in which each of R^{1a}, R^{3a}, and R^{5b} has a ring structure is a preferred form. In addition, in the case where the number of ring structures is two, a compound in which each of R^{1a} and R^{3a} has a ring structure or a compound in which each of R^{1a} and R^{5b} has a ring structure is a preferred form.

With regard to the ring structure, those described in R^{5b} are preferable. Among these, those having a ring as a group selected from a cyclohexyl group, a phenyl group, and a benzyl group are preferable.

Meanwhile, the compound represented by formula (I) according to the present invention may have not only one but also a plurality of barbituric acid skeletons, in the molecule.

The number of barbituric acid skeletons is preferably 1 to 5, more preferably 1 to 3, further preferably 1 or 2, and particularly preferably 1.

It is preferable to contain the barbituric acid skeleton into a substituent in each of the defined groups in R¹, R^{3a}, R^{5a}, or R^{5b} in formula (I), respectively.

The following structures specifically illustrate the preferred structure of the portion other than R^{5a} of the compound represented by formula (I) according to the present invention.

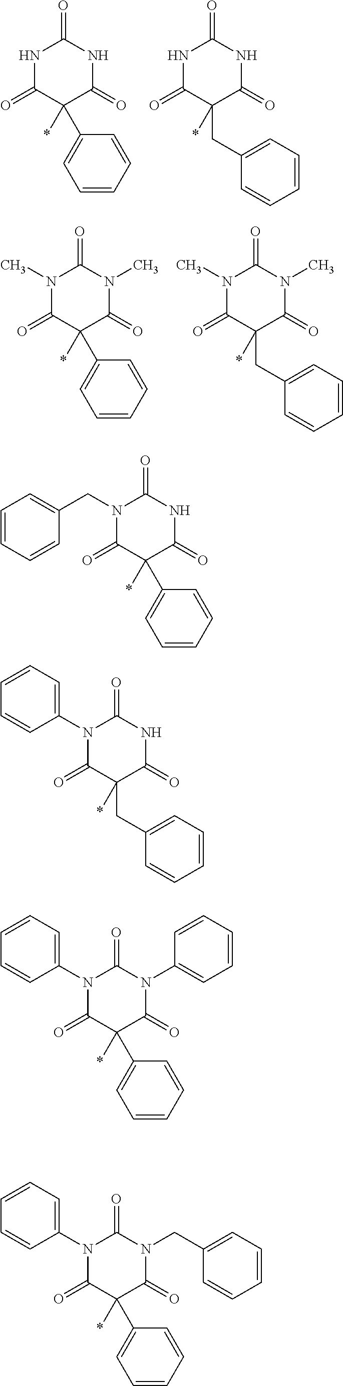

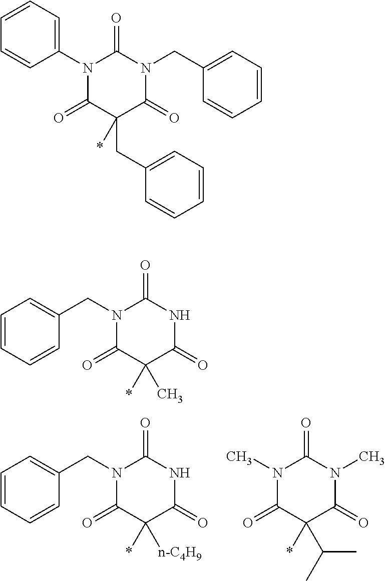

Herein, bonding to R^{5a} occurs at * portion.

### <Compound Represented by Formula (II)>

In the present invention, the compound represented by formula (I) is particularly preferably a compound represented by formula (II).

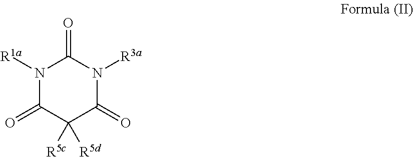

In formula (II), R^{1a} and R^{3a} each independently represent a hydrogen atom, an alkyl group, an alkenyl group, a cycloalkyl group, a cycloalkenyl group, an aryl group, or a heterocyclic group. R^{5c} represents a hydroxy group. R^{5d} represents an alkyl group, an alkenyl group, a cycloalkyl group, a cycloalkenyl group, or an aryl group. Each group of R^{1a}, R^{3a} and R^{5b} may be substituted with a substituent. Herein, one or more ring structures are contained in R^{1a}, R^{3a}, and R^{5d} in total.

Herein, R^{1a} and R^{3a} have the same meanings as R^{1a} and R^{3a} in formula (I), respectively; and preferable ranges are also the same. R^{5d} has the same meaning as the alkyl group, the alkenyl group, the cycloalkyl group, the cycloalkenyl group, or the aryl group, each of which is mentioned as the preferred group in R^{5b} in formula (I), and the preferred range thereof is also the same as the corresponding group of R^{5b}. The number of ring structures in R^{1a}, R^{3a}, and R^{5d} is preferably 2 or more, and more preferably 2 or 3.

Hereinafter, specific examples of the compound represented by formula (I) according to the present invention are shown, but the present invention is not limited thereto.

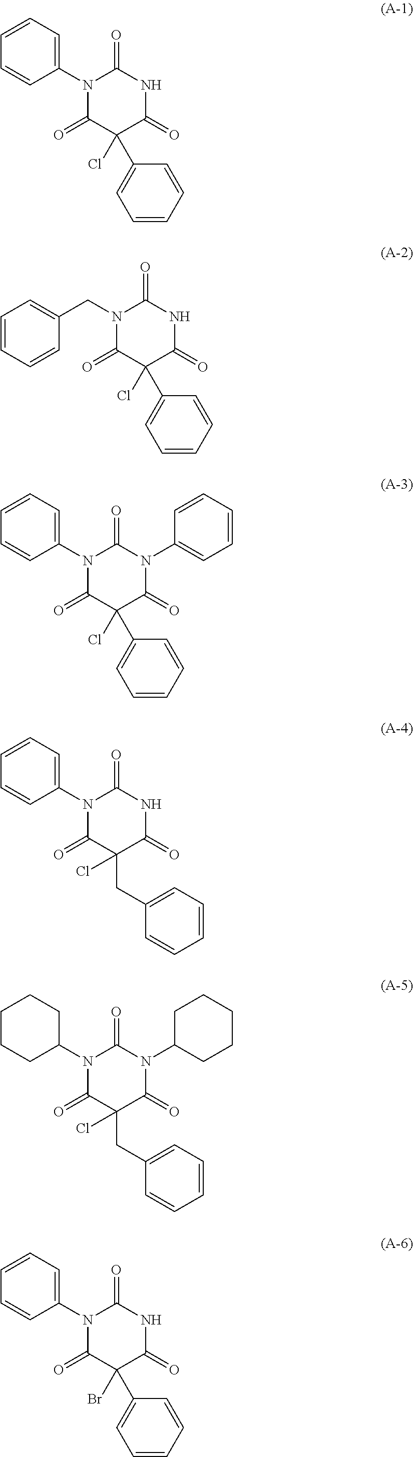

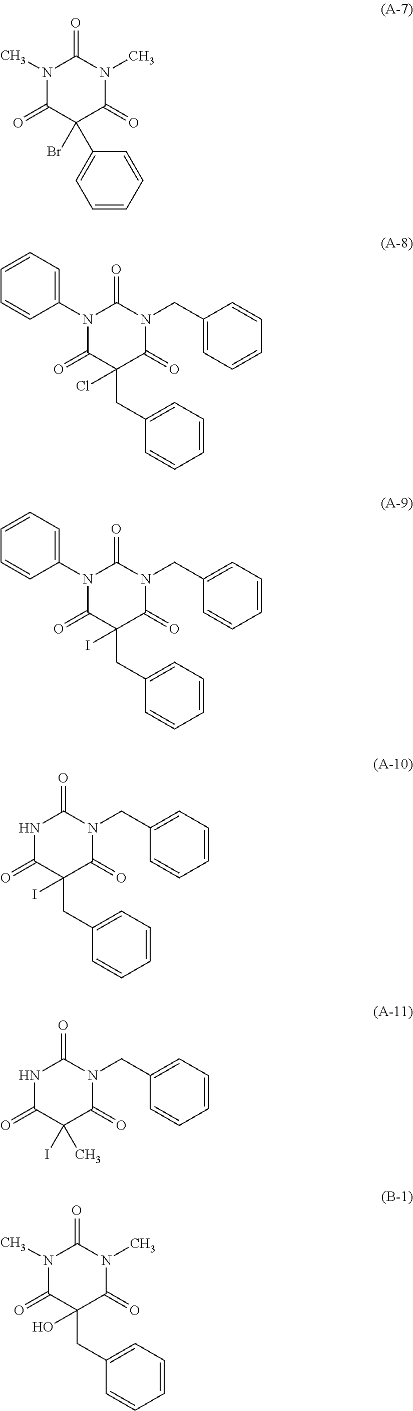 [SMILES: CN1C(=O)N(C)C(=O)C(Br)(c2ccccc2)C1=O ; O=C1N(Cc2ccccc2)C(=O)C(Cl)(Cc2ccccc2)C(=O)N1c1ccccc1 ; O=C1N(Cc2ccccc2)C(=O)C(I)(Cc2ccccc2)C(=O)N1c1ccccc1 ; O=C1NC(=O)C(I)(Cc2ccccc2)C(=O)N1Cc1ccccc1 ; CC1(I)C(=O)NC(=O)N(Cc2ccccc2)C1=O ; CN1C(=O)N(C)C(=O)C(O)(Cc2ccccc2)C1=O]

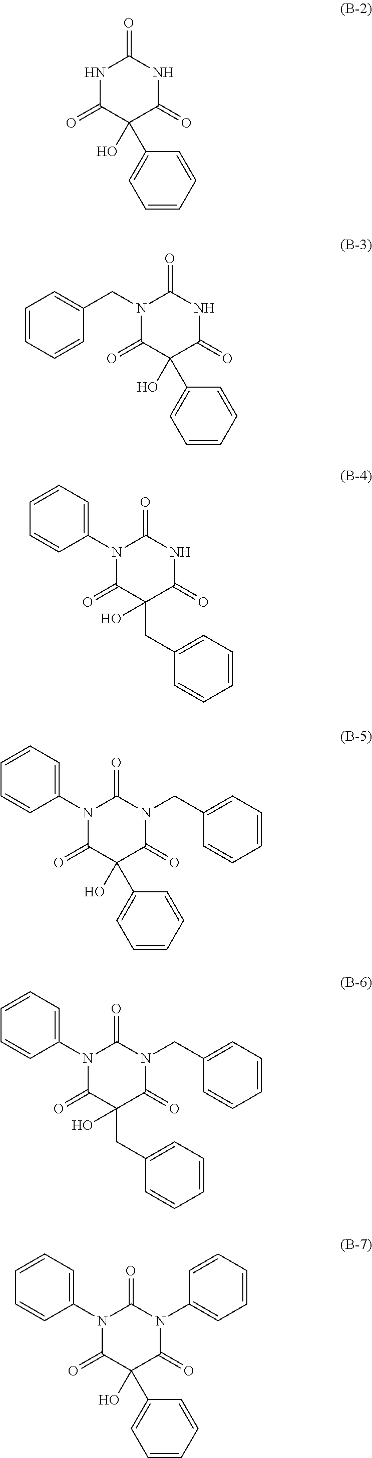 [SMILES: O=C1NC(=O)C(O)(c2ccccc2)C(=O)N1 ; O=C1NC(=O)C(O)(c2ccccc2)C(=O)N1Cc1ccccc1 ; O=C1NC(=O)C(O)(Cc2ccccc2)C(=O)N1c1ccccc1 ; O=C1N(Cc2ccccc2)C(=O)C(O)(c2ccccc2)C(=O)N1c1ccccc1 ; O=C1N(Cc2ccccc2)C(=O)C(O)(Cc2ccccc2)C(=O)N1c1ccccc1 ; O=C1N(c2ccccc2)C(=O)C(O)(c2ccccc2)C(=O)N1c1ccccc1]

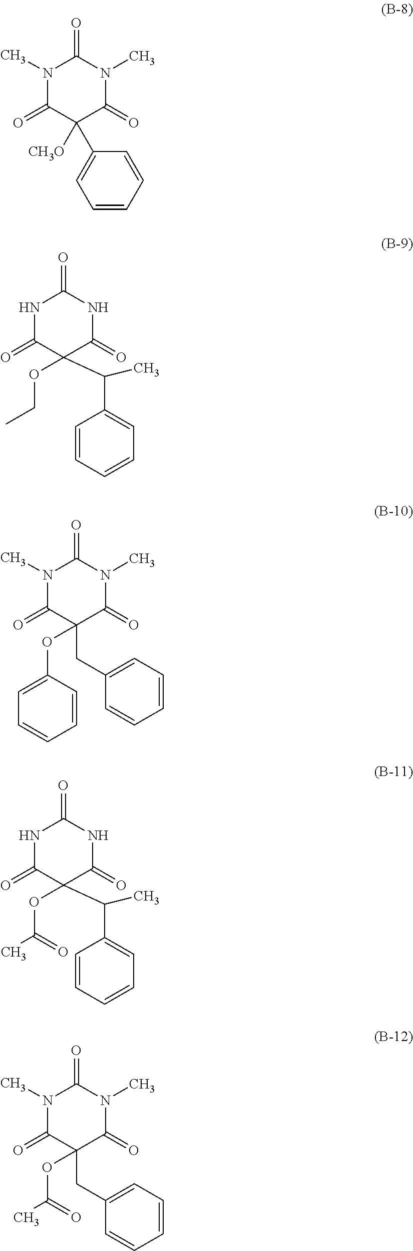

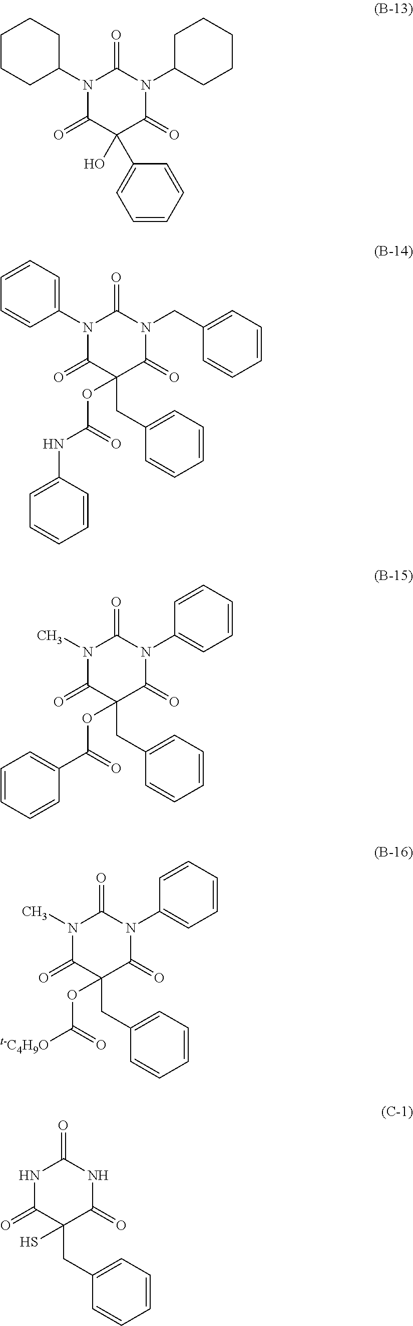

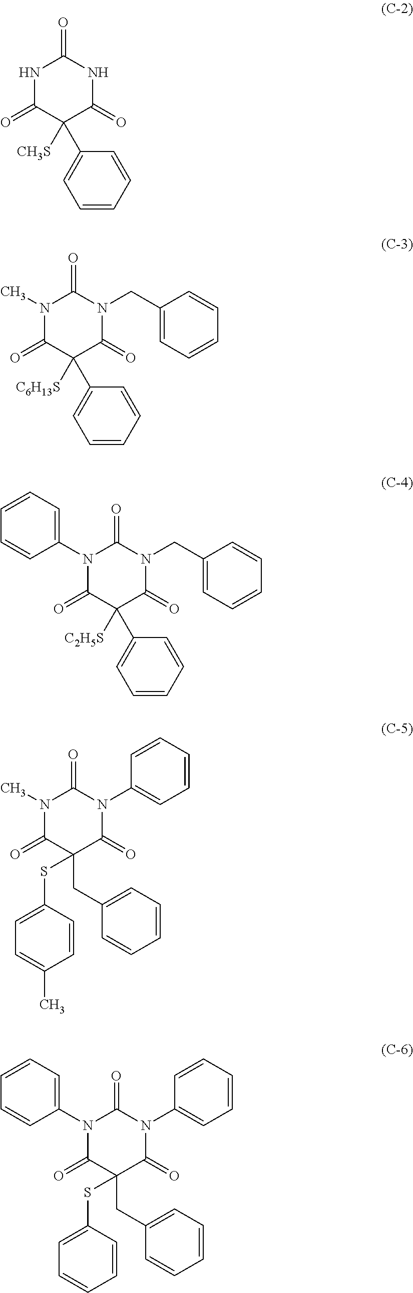

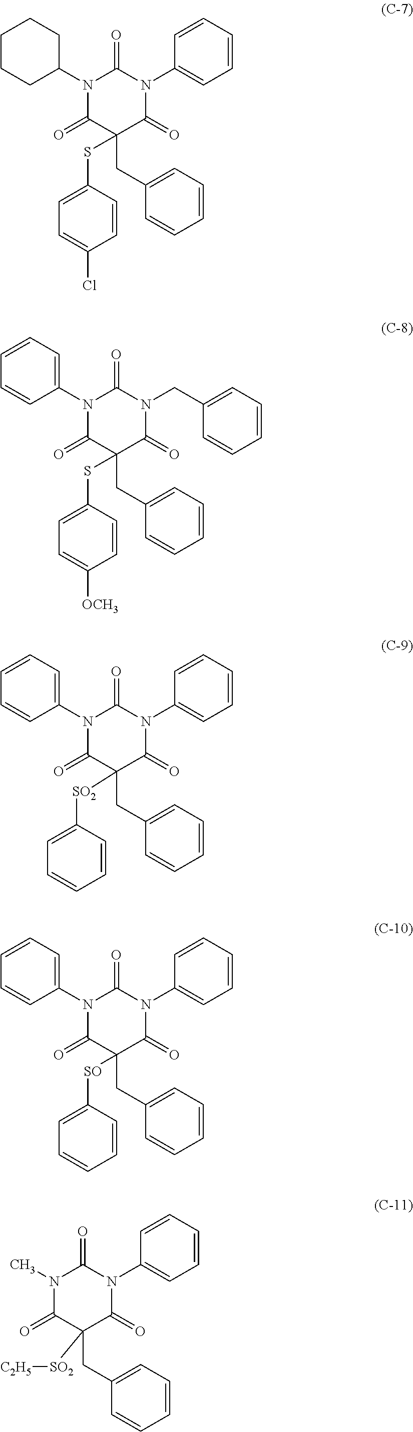

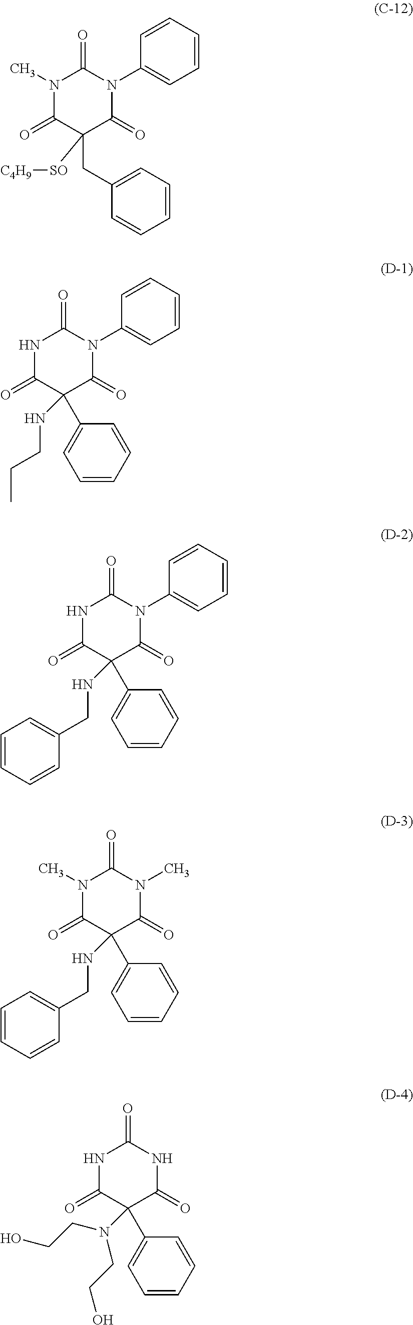

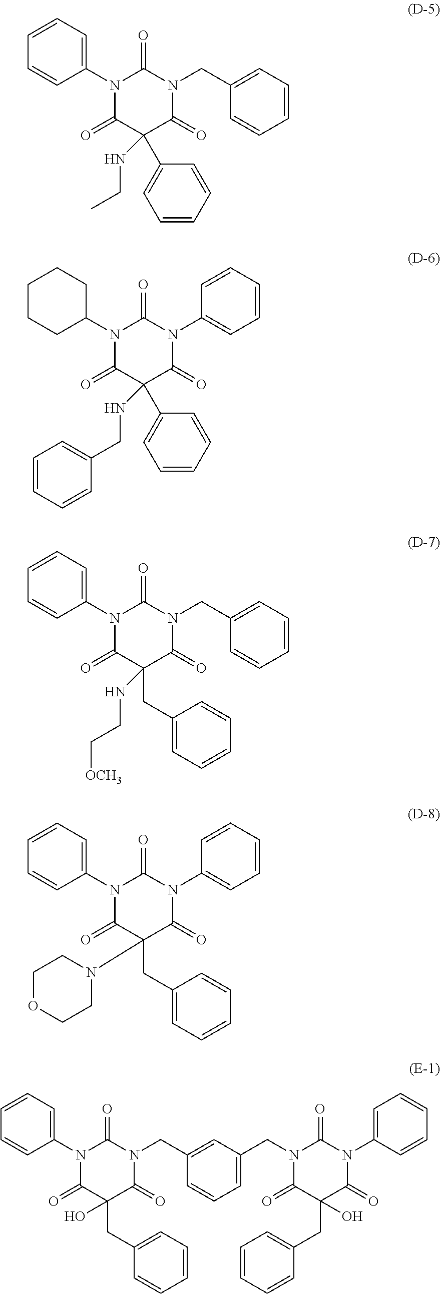

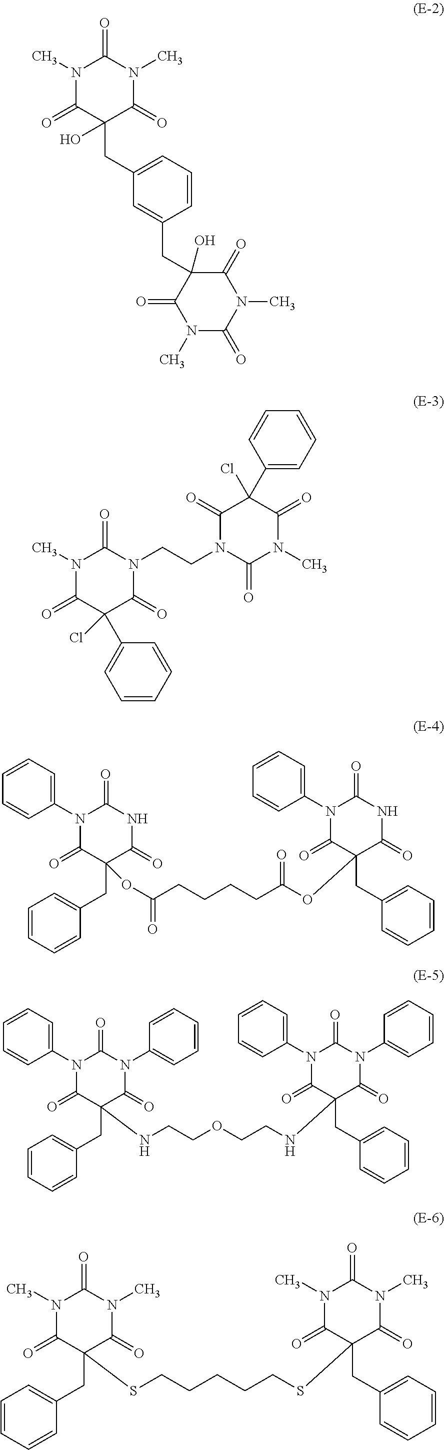 [SMILES: CN1C(=O)N(C)C(=O)C(O)(Cc2cccc(CC3(O)C(=O)N(C)C(=O)N(C)C3=O)c2)C1=O ; CN1C(=O)N(CCN2C(=O)N(C)C(=O)C(Cl)(c3ccccc3)C2=O)C(=O)C(Cl)(c2ccccc2)C1=O ; O=C(CCCCC(=O)OC1(Cc2ccccc2)C(=O)NC(=O)N(c2ccccc2)C1=O)OC1(Cc2ccccc2)C(=O)NC(=O)N(c2ccccc2)C1=O ; O=C1N(c2ccccc2)C(=O)C(Cc2ccccc2)(NCCOCCNC2(Cc3ccccc3)C(=O)N(c3ccccc3)C(=O)N(c3ccccc3)C2=O)C(=O)N1c1ccccc1 ; CN1C(=O)N(C)C(=O)C(Cc2ccccc2)(SCCCCCSC2(Cc3ccccc3)C(=O)N(C)C(=O)N(C)C2=O)C1=O]

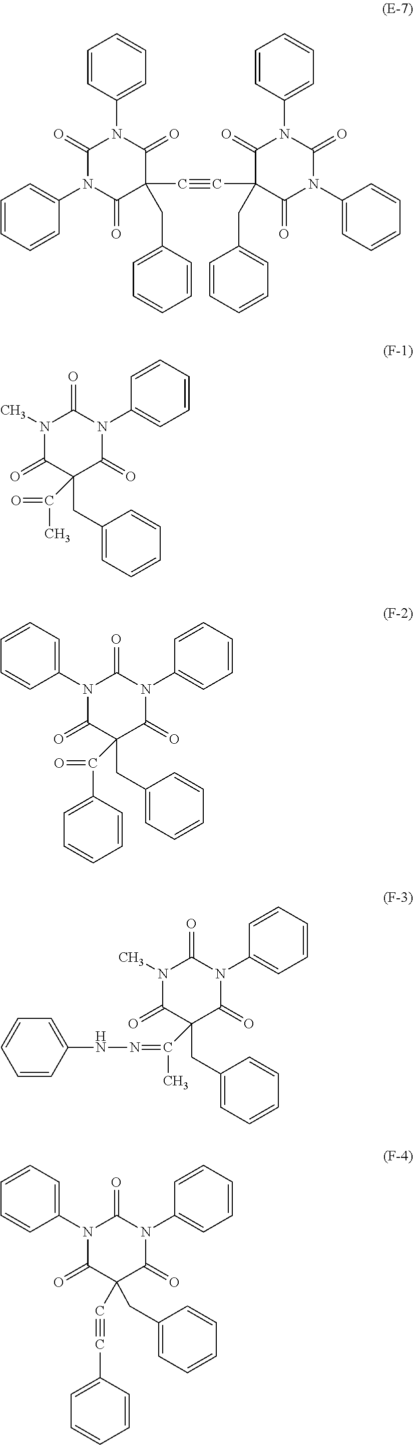 [SMILES: O=C1N(c2ccccc2)C(=O)C(C#CC2(Cc3ccccc3)C(=O)N(c3ccccc3)C(=O)N(c3ccccc3)C2=O)(Cc2ccccc2)C(=O)N1c1ccccc1 ; CC(=O)C1(Cc2ccccc2)C(=O)N(C)C(=O)N(c2ccccc2)C1=O ; O=C1N(c2ccccc2)C(=O)C(Cc2ccccc2)(C(=O)c2ccccc2)C(=O)N1c1ccccc1 ; CC(=NNc1ccccc1)C1(Cc2ccccc2)C(=O)N(C)C(=O)N(c2ccccc2)C1=O ; O=C1N(c2ccccc2)C(=O)C(C#Cc2ccccc2)(Cc2ccccc2)C(=O)N1c1ccccc1]

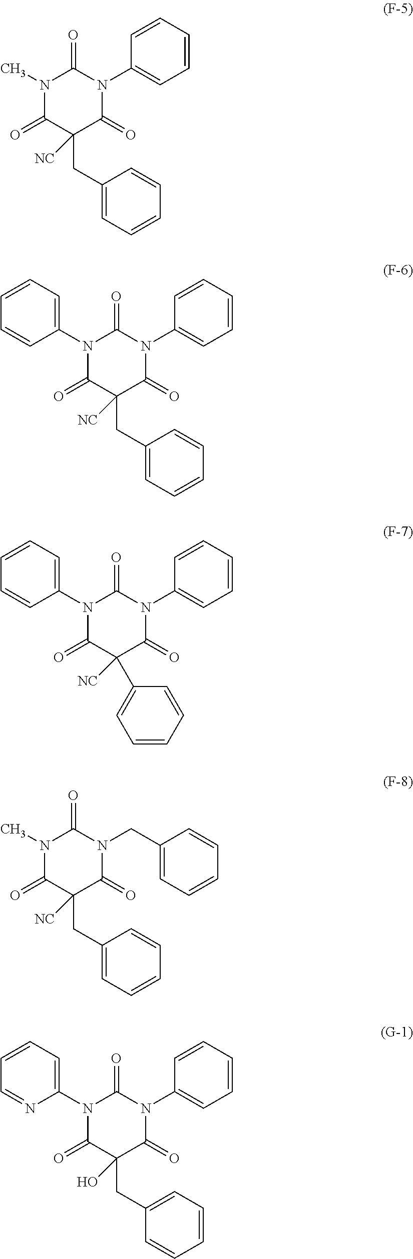 [SMILES: CN1C(=O)N(c2ccccc2)C(=O)C(C#N)(Cc2ccccc2)C1=O ; N#CC1(Cc2ccccc2)C(=O)N(c2ccccc2)C(=O)N(c2ccccc2)C1=O ; N#CC1(c2ccccc2)C(=O)N(c2ccccc2)C(=O)N(c2ccccc2)C1=O ; CN1C(=O)N(Cc2ccccc2)C(=O)C(C#N)(Cc2ccccc2)C1=O ; O=C1N(c2ccccc2)C(=O)C(O)(Cc2ccccc2)C(=O)N1c1ccccn1]

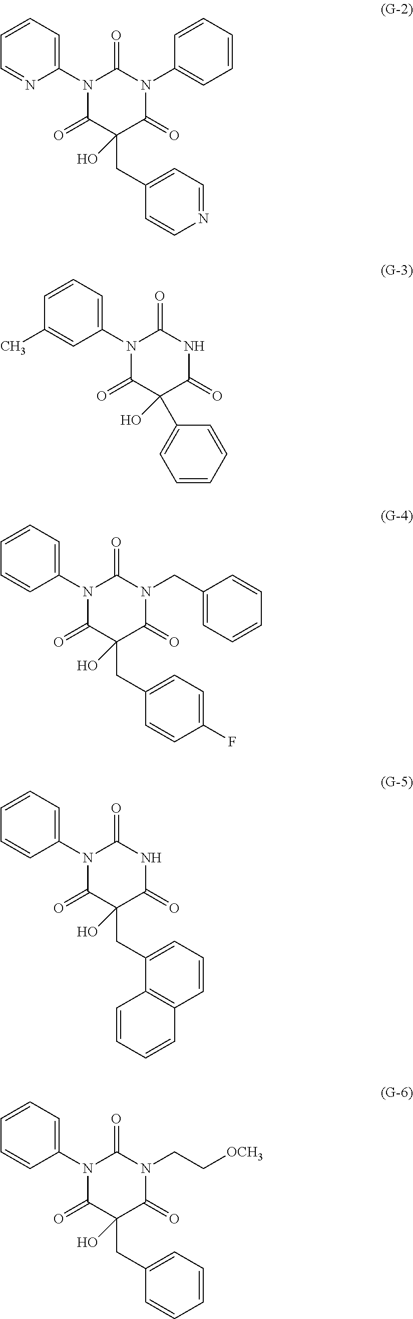 [SMILES: O=C1N(c2ccccc2)C(=O)C(O)(Cc2ccncc2)C(=O)N1c1ccccn1 ; Cc1cccc(N2C(=O)NC(=O)C(O)(c3ccccc3)C2=O)c1 ; O=C1N(Cc2ccccc2)C(=O)C(O)(Cc2ccc(F)cc2)C(=O)N1c1ccccc1 ; O=C1NC(=O)C(O)(Cc2cccc3ccccc23)C(=O)N1c1ccccc1 ; COCCN1C(=O)N(c2ccccc2)C(=O)C(O)(Cc2ccccc2)C1=O]

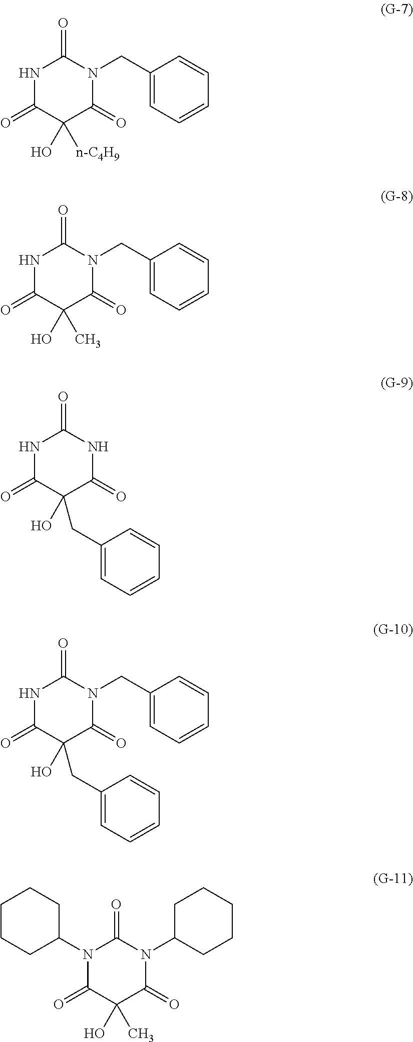

The compound represented by formula (I) is known to be able to synthesize by a method of synthesizing barbituric acid, based on condensation of a urea derivative with a malonic acid derivative. The barbituric acid having two substituents on the N atoms may be obtained, by heating a N,N′-disubstituted urea with malonyl chloride, or by heating with a combination of malonic acid and an activator, such as acetic anhydride. For example, methods described in: Journal of the American Chemical Society, vol. 61, p. 1015 (1939), Journal of Medicinal Chemistry, vol. 54, p. 2409 (2011), Tetrahedron Letters, vol. 40, p. 8029 (1999), and WO2007/150011, are preferably used.

Herein, both unsubstituted and substituted malonic acids are acceptable for use in the condensation. By using malonic acid having any of correspondent substituents for R⁵ so as to configure barbituric acid, the compounds represented by formula (I) according to the present invention can be synthesized. Further, the compound represented by formula (I) can be synthesized, alternatively by subjecting the barbituric acid unsubstituted at the 5-position, which is obtainable by condensing unsubstituted malonic acid with an urea derivative, to a nucleophilic substitution reaction, a Michael addition reaction, or the like.

The methods preferably used herein are described, for example, in Organic Letters, vol. 5, p. 2887 (2003), Journal of Medicinal Chemistry, vol. 17, p. 1194 (1974), and Journal of Organic Chemistry, vol. 68, p. 4684 (2003).

Methods of synthesizing the compound represented by formula (I) according to the present invention are not limited to those described above.

Although the content of the compound represented by formula (I) in the cellulose acylate film is not particularly limited, the content is preferably 0.001 to 50 parts by mass, more preferably 0.005 to 30 parts by mass, and particularly preferably 0.01 to 15 parts by mass, with respect to 100 parts by mass of cellulose acylate. By having such a content, the hardness and the suppression of the optical film coloration, which are the effects of the present invention, can be sufficiently exerted, and further, the transparency of the film is also maintained.

In the cellulose acylate film, in the case where two or more compounds represented by formula (I) are contained, it is preferable that the total amount thereof is within the range.

In the present invention, the combination use of a barbituric acid compound having a structure other than the compound represented by formula (I), may also be mentioned as a preferred aspect.

Such a compound is preferably a compound represented by formula (A).

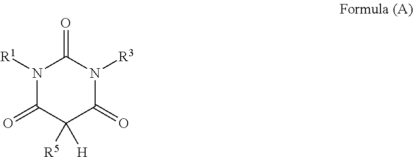

In formula (A), R¹ and R³ each independently represent a hydrogen atom, an alkyl group, an alkenyl group, a cycloalkyl group, a cycloalkenyl group, an aryl group, or a heterocyclic group; and R⁵ represents a hydrogen atom or a substituent. Each group of R¹, R³ and R⁵ may be substituted with a substituent.

R¹, R³ and R⁵ have the same meaning as R^{1a}, R^{3a} and R^{5b} in formula (I), and preferable ranges are also the same.

The sum of the ring structures present in R¹, R³, and R⁵ in the compound represented by formula (A) is preferably 1 or more, and more preferably from 1 to 6. In addition, the sum is still more preferably 2 or more, and particularly preferably from 2 to 6. Among them, the sum is preferably from 2 to 4, and most preferably 3.

Among these, a compound in which each of R¹, R³, and R⁵ has a ring structure is a preferred form.

Herein, in the case where the compound represented by formula (I) is used in combination with the compound represented by formula (A), the combination is preferable in which the structures of R¹ and R^{1a} are the same as each other and the structures of R³ and R^{3a} are the same as each other.

Meanwhile, herein, to be the same as each other means that, for example, when R¹ is a methyl group, R^{1a} is also a methyl group.

In the present invention, in addition to the above, the combination is particularly preferable in which the structures of R⁵ and R^{5b} are also the same as each other.

Hereinafter, specific examples of the compound represented by formula (A) are shown, but the present invention is not limited thereto.

Further, it is preferable that any of compounds described in JP-A-2011-118135 and JP-A-2011-126968 is also used in combination with the compound represented by formula (I).

Herein, “Ph” represents a phenyl group, “cHex” represents a cyclohexyl group, and “C₆H₄” represents a phenylene group. A group of ( ), such as C₆H₄(p-CH₃), represents a substituent to the phenyl group, and “p-” indicates that the group is at the p-position.

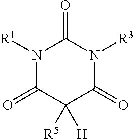

| Compound No. | R¹ | R³ | R⁵ |
|---|---|---|---|
| BA-1 | H | H | Ph |
| BA-2 | H | H | CH₂Ph |
| BA-3 | H | H | CHPh₂ |
| BA-4 | H | H | CH₂C₆H₄(p-CH₃) |
| BA-5 | H | H | CH₂C₆H₄(p-OCH₃) |
| BA-6 | H | H | CH₂C₆H₄(p-Cl) |
| BA-7 | CH₃ | CH₃ | Ph |
| BA-8 | CH₃ | CH₃ | CH₂Ph |
| BA-9 | H | Ph | Ph |
| BA-10 | H | CH₂Ph | Ph |
| BA-11 | H | Ph | CH₂Ph |
| BA-12 | H | CH₂Ph | CH₂Ph |
| BA-13 | H | CHPh₂ | Ph |
| BA-14 | H | cHex | cHex |
| BA-15 | Ph | Ph | Ph |
| BA-16 | Ph | Ph | CH₂Ph |
| BA-17 | Ph | Ph | n-C₄H₉ |
| BA-18 | Ph | Ph | CH(CH₃)Ph |
| BA-19 | Ph | C₆H₄(p-CH₃) | Ph |
| BA-20 | Ph | C₆H₄(p-OCH₃) | CH₂Ph |
| BA-21 | Ph | CH₂Ph | CH₂CH₂Ph |
| BA-22 | Ph | CH₂Ph | Ph |
| BA-23 | Ph | CH₂Ph | CH₂Ph |
| BA-24 | cHex | cHex | Ph |
| BA-25 | cHex | cHex | CH₂Ph |
| BA-26 | cHex | cHex | cHex |
| BA-27 | CH₂Ph | CH₂Ph | Ph |
| BA-28 | CH₂Ph | CH₂Ph | CH₂Ph |
| BA-29 | CH₂Ph | CH₂Ph | n-C₄H₉ |
| BA-30 | Ph | Ph | CH₂CH₂CN |
| BA-31 | Ph | Ph | CH₂CH₂COOC₂H₅ |
| BA-32 | Ph | CH₂CH₂OCH₃ | Ph |
| BA-33 | Ph | CH₂CH₂COOC₂H₅ | CH₂Ph |
| BA-34 | Ph | CH₂CH₂OH | CH₂Ph |
| BA-35 | CH₃ | CH₃ | n-C₄H₉ |
| BA-36 | H | CH₂Ph | n-C₄H₉ |
| BA-37 | H | CH₂Ph | CH₃ |

The compound represented by formula (A) can be synthesized according to the synthetic method of the compound represented by formula (I), and the documents cited in the description of the synthetic method of the compound represented by formula (I) can be applied to as it is.

The sum of the contents of the compound represented by formula (I) and the compound represented by formula (A) is preferably from 0.1 to 50 parts by mass, more preferably from 0.2 to 30 parts by mass, still more preferably 0.3 to 15 parts by mass, and particularly preferably from 0.3 to 10 parts by mass, with respect to 100 parts by mass of cellulose acylate.

### <Cellulose Acylate>

In the present invention, cellulose acylate is used as a main component of the cellulose acylate film. One kind of cellulose acylate may be used, or alternatively two or more kinds thereof may be used. For example, the cellulose acylate may be a cellulose acylate having only an acetyl group as the acyl substituent thereof; a cellulose acylate having a plurality of different acyl substituents as the acyl substituent thereof may be used; or alternatively, the cellulose acylate may be a mixture of cellulose acylates that are different from one another.

The cellulose material for cellulose acylate which is used in this invention includes cotton liter and wood pulp (hardwood pulp, softwood pulp), and cellulose acylate obtained from any such cellulose material are usable herein. Those cellulose material may be mixed for use herein. The cellulose materials are described in detail, for example, by Marusawa & Uda's in “Plastic Material Lecture (17), Cellulose Resin” by Nikkan Kogyo Shinbun (1970) and Hatsumei Kyokai's Disclosure Bulletin 2001-1745 (pp. 7-8), and those celluloses described therein may be usable herein.

In the present specification, the acyl group of the cellulose acylate may be one kind, or two or more kinds of acyl groups. It is preferable that the cellulose acylate used in the present invention has an acyl group having 2 or greater carbon atoms as a substituent. The acyl group having 2 or greater carbon atoms is not particularly limited such that it may be an aliphatic acyl group or an aromatic acyl group. Examples thereof include cellulosic alkylcarbonyl groups, alkenylcarbonyl groups, aromatic carbonyl groups, and aromatic alkylcarbonyl groups, each of which may have a substituted group. Preferable examples thereof include acetyl, propionyl, butanoyl, heptanoyl, hexanoyl, octanoyl, decanoyl, dodecanoyl, tridecanoyl, tetradecanoyl, hexadecanoyl, octadecanoyl, isobutanoyl, tert-butanoyl, cyclohexane carbonyl, oleoyl, benzoyl, naphthyl carbonyl, and cinnamoyl. Among these, acetyl, propionyl, butanoyl, decanoyl, octadecanoyl, tert-butanoyl, oleoyl, benzoyl, naphthyl carbonyl, and cinnamoyl are more preferred. Further, acetyl, propionyl and butanoyl are preferred.

It is preferable that the cellulose acylate used in the present invention has an acyl group having 2 to 4 carbon atoms as a substituent. When two or more kinds of acyl groups are used, it is preferable that one kind of the acyl groups is an acetyl group, and another kind of the acyl group having 2 to 4 carbon atoms is preferably propionyl group or butyryl group. By use of these cellulose acylates, a solution with a good solubility can be prepared. Especially in a non-chlorine organic solvent, preparation of a good solution becomes possible. Further, preparation of a solution having a low viscosity and a good filterability becomes possible.

In the present invention, particularly, a cellulose acylate having one acetyl group as the acyl group is preferably used, due to its excellent in hardness improvement effects by the compound represented by formula (I).

Hereinafter, cellulose acylate preferably used in the present invention is described in detail.

The glucose unit having β-1, β-4 bonds, which constitutes cellulose, has free hydroxy groups at the 2-, 3-, and 6-positions thereof. The cellulose acylate is a polymeric substance (polymer) in which a part of or all of these hydroxy groups is or are acylated.

The acyl substitution degree indicates a degree of acylation of the hydroxy groups located at the 2-, 3-, and 6-positions of cellulose. When each of the hydroxy groups at the 2-, 3-, and 6-positions of all of the glucose units is acylated, the total acyl substitution degree is 3. For example, when each of the hydroxy groups only at the 6-position of all of the glucose units is acylated, the total acyl substitution degree is 1. In the same manner, even if each of the hydroxy groups at either the 6- position or the 2-position of all of the glucose unit is acylated, the total acyl substitution degree is 1.

That is to say, the acyl substitution degree indicates a degree of acylation, provided that when all of the hydroxy groups of the glucose molecule are entirely acylated, the acyl substitution degree is 3.

The details of the measurement method of the acyl substitution degree are described by Tezuka, et al. in Carbohydrate, Res., 273 (1995) p. 83 to 91. The acyl substitution degree can be determined according to the method defined in ASTM-D817-96.

When the total acyl substitution degree of the cellulose acylate used in the present invention is A, the A is preferably from 1.5 to 3.0 (1.5≦A≦3.0), more preferably from 2.0 to 2.97, still more preferably from 2.5 to less than 2.97, and particularly preferably from 2.70 to 2.95.

When the acyl group of the cellulose acylate used in the present invention is only an acetyl group, if we take the total acetyl substitution degree is B, the B is preferably from 2.0 to 3.0 (2.0≦B≦3.0), more preferably from 2.0 to 2.97, still more preferably from 2.5 to less than 2.97, especially preferably from 2.55 to less than 2.97, more specially preferably from 2.60 to 2.96, and most preferably from 2.70 to 2.95.

Meanwhile, the effects of the compound represented by formula (I) according to the present invention is exerted particularly, with respect to the cellulose acylate in which A that is the total degree of acyl substitution is more than 2.00.

In the case where the cellulose acylate film of the optical film of the present invention is a laminate (multilayer configuration), the degree of acyl group substitution of the cellulose acylate in each layer may be uniform, or a plurality of cellulose acrylates which have different degrees of acyl group substitution or different acyl groups may be present in one layer in a mixed manner, in the cellulose acylate film.

In the case where an acid anhydride or an acid chloride is used as an acylating agent in acylation of the cellulose, methylene chloride or an organic acid, for example, acetic acid and the like, is used as an organic solvent which acts as a reaction solvent.

As for the catalyst, when the acylating agent is an acid anhydride, a protic catalyst, such as sulfuric acid, is preferably used. While, when the acylating agent is an acid chloride (for example, CH₃CH₂COCl), a basic compound is used.

A most common industrial method for the synthesis of a mixed fatty acid ester of cellulose, is a method of acylating cellulose with a mixed organic acid component that includes fatty acids corresponding to an acetyl group and to any other acyl group (acetic acid, propionic acid, valeric acid, and the like) or their acid anhydrides.

The cellulose acylate may be produced, for example, according to the method described in JP-A-10-45804.

In the film of the present invention, especially in the cellulose acylate film used in the present invention, it is the proportion of preferably from 5 to 99% by mass, more preferably from 20 to 99% by mass, and particularly preferably from 50 to 95% by mass, of the cellulose acylate, with respect to the total solid content of the film, from the viewpoint of water-vapor transmission ratio.

### <Other Additives>

To the optical film of the present invention, especially to the cellulose acylate film, a retardation-controlling agent (retardation-developing agent and retardation-reducing agent), and, as a plasticizer, a polycondensation ester compound (polymer), and a polyvalent ester of polyvalent alcohol, for example, a phthalic acid ester, a phosphoric acid ester, and the like, and further additives, such as a ultraviolet absorber, an antioxidant, and a matting agent, may be added.

In the present specification, when compound groups are described, they may be described incorporating therein the expression“-based”, for example, like a phosphoric acid ester-based compound. However, in this case, this means the same as the phosphoric acid ester compound.

### (Retardation-Reducing Agent)

In the present invention, as a retardation-reducing agent, a phosphoric acid ester-based compound, and a compound other than the non-phosphoric acid ester-based compound known as an additive for the cellulose acylate film, may be generally adopted.

The polymer retardation-reducing agent is selected from a phosphoric acid polyester-based polymer, a styrene-based polymer, an acrylic-based polymer, and their copolymers. Of these, an acrylic-based polymer and a styrene-based polymer are preferred. Further, at least one polymer having a negative intrinsic birefringence, such as a styrene-based polymer and an acrylic-based polymer, is preferably contained.

A low-molecular retardation-reducing agent that is the compound other than the non-phosphoric acid ester-based compound, is described below. These compounds may be a solid or an oily matter. That is, their melting point and boiling point are not particularly limited. For example, they may be a mixture of an ultraviolet absorber of 20° C. or lower and an ultraviolet absorber of 20° C. or higher, or a mixture of degradation inhibitors in the same manner. Further, an infrared-absorbing dye is described in, for example, JP-A-2001-194522. Further, as for the timing for their addition, additives may be added at any time in preparation processes of a cellulose acylate solution (dope). The dope preparation may be performed by incorporating the preparation process in which an additive is added, into the last preparation process of the dope preparation processes. Further, the addition amount of each material is not particularly limited, as long as their functions are exhibited.

The low-molecular retardation-reducing agent that is the compound other than the non-phosphoric acid ester-based compound is not particularly limited. Details thereof are described in paragraphs [0066] to [0085] of JP-A-2007-272177.

The compound represented by formula (1) as described in the paragraphs

to [0085] of JP-A-2007-272177 can be obtained by a condensation reaction of a sulfonyl chloride derivative and an amine derivative, as described in the above publication.

The compound represented by formula (2) described in JP-A-2007-272177 can be obtained, using a condensation agent (for example, dicyclohexylcarbodiimide (DCC), and the like), by a dehydration condensation reaction of a carboxylic acid and an amine, a substitution reaction of a carboxylic acid chloride derivative and an amine derivative, or the like.

It is more preferable, from the viewpoint of realizing an Nz factor, that the retardation-reducing agent is an Rth reducing agent. Of the retardation-reducing agents, examples of the Rth reducing agent include: an acrylic-based polymer, a styrene-based polymer, and also a low-molecular compound represented by any one of formulae (3) to (7) described in JP-A-2007-272177. Among them, an acrylic-based polymer and a styrene-based polymer are preferred, and an acrylic-based polymer is more preferred.

The content of the retardation-reducing agent is preferably set to the proportion of from 0.01 to 30% by mass, more preferably from 0.1 to 20% by mass, and particularly preferably from 0.1 to 10% by mass, with respect to the cellulosic resin. When the addition amount is set to 30% by mass or less, compatibility with the cellulosic resin can be improved, whereby the resultant film which has excellent transparency can be produced. When two or more retardation-reducing agents are used, it is preferable that the total amount thereof is within the range.

### (Retardation-Developing Agent)

The optical film of the present invention may contain at least one retardation-developing agent, in order to develop a value of retardation.

The retardation-developing agent is not particularly limited, and examples thereof include a material including a stick-shaped or disc-shaped compound, and a compound that shows retardation-developing property of the non-phosphoric acid ester-based compounds. As for the stick-shaped or disc-shaped compound, a compound having at least two aromatic rings can be preferably used as the retardation-developing agent.

The content of the retardation-developing agent composed of a stick-shaped compound is preferably from 0.1 to 30 parts by mass, and more preferably from 0.5 to 20 parts by mass, with respect to 100 parts by mass of the polymer component including cellulose acylate.

The disc-shaped compound, when compared to the stick-shaped compound, is excellent in Rth retardation-developing property, and therefore preferably used in the case where particularly large Rth retardation is required. Two or more retardation-developing agents may be used in combination.

The retardation-developing agent preferably has a maximum absorption in the wavelength region of from 250 to 400 nm, and preferably it has substantially no absorption in the visible region.

The details of the retardation-developing agent are described on page 49 of Journal of Technical Disclosure 2001-1745.

The content of the retardation-developing agent composed of a disc-shaped compound is preferably from 0.1 to 30 parts by mass, and more preferably from 0.5 to 20 parts by mass, with respect to 100 parts by mass of the polymer component including cellulose acylate.

The content of the disc-shaped compound that is contained in the retardation-developing agent is preferably less than 3 parts by mass, more preferably less than 2 parts by mass, and particularly preferably less than 1 part by mass, with respect to 100 parts by mass of cellulose acylate.

### [Plasticizer (Hydrophobizing Agent)]

In the optical film, particularly the cellulose acylate film, the water content or the water-vapor transmission ratio of the cellulose acylate film decreases when a plasticizer is contained in the cellulose acylate. Thus, the hydrolysis reaction of the cellulose acylate due to the moisture in the cellulose acylate film is suppressed. Further, the plasticizer makes it possible to suppress diffusion of additives in the cellulose acylate film to a polarizer layer under the conditions of high temperature and high humidity, whereby deterioration of polarizer properties can be improved.

The compound represented by formula (I) according to the present invention can also be used as a plasticizer, by being contained in the optical film, particularly the cellulose acylate film. In other words, it is possible to enhance the hardness of the cellulose acylate film, at the same time to control the glass transition temperature and to obtain the effects of improving the durability including the decreases in the water content and the water-vapor transmission ratio, as described above. Furthermore, the compound represented by formula (I) according to the present invention can exert the effects of enhancing the hardness, even in the case of using it with another plasticizer, in combination with the compound of formula (I). Thus, a plurality of plasticizers may be contained in the optical film, particularly in the cellulose acylate film, in combination with the compound of formula (I).

In the present invention, among the plasticizers in combination with the compound of formula (I), a polyvalent ester-based plasticizer is preferable in which ester groups are close in position in the molecule so as to clog. Specific examples of the polyvalent ester-based plasticizer include a polycondensation ester compound (hereinafter, referred to as “polycondensation ester-based plasticizer”), a polyvalent ester of polyvalent alcohol (hereinafter, referred to as “polyvalent alcohol ester-based plasticizer”), and a carbohydrate compound (hereinafter, referred to as “carbohydrate derivative-based plasticizer”). In the present invention, these compounds are excellent in exertion of the plasticizer effects as described above.

Hereinafter, plasticizers for use in the present invention are described in detail. (Polycondensation ester-based plasticizer)

The cellulose acylate film of the present invention also preferably contains a polycondensation ester-based plasticizer. By containing the polycondensation ester-based plasticizer, it is possible to achieve a cellulose ester film excellent in humidity stability and a polarizing plate excellent in durability.

The polycondensation ester-based plasticizer is obtained by bringing a divalent carboxylic acid compound and a diol compound into polycondensation.

The polycondensation ester-based plasticizer is preferably obtained by bringing at least one dicarboxylic acid represented by formula (a) and at least one diol represented by formula (b) into polycondensation.

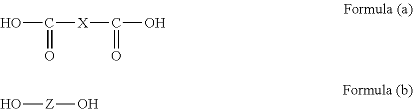

In formulae (a) and (b), X represents a divalent aliphatic group having 2 to 18 carbon atoms, a divalent aromatic group having 6 to 18 carbon atoms, or a divalent heterocyclic group having 2 to 18 carbon atoms; and Z represents a divalent aliphatic group having 2 to 8 carbon atoms.

Examples of the divalent carboxylic acid compound represented by formula (a) include an aliphatic carboxylic acid, an aromatic or heterocyclic carboxylic acid as described above, and an aliphatic carboxylic acid or an aromatic carboxylic acid is preferable.

On the other hand, examples of the diol compound include an aromatic or heterocyclic compound, in addition to the aliphatic compound represented by formula (b) above.

Out of these, the polycondensation ester-based plasticizer is preferably obtained from at least one dicarboxylic acid having an aromatic ring (also be called an aromatic dicarboxylic acid) and at least one aliphatic diol having the average carbon number of from 2.5 to 8.0. Further, it is also preferable that the dicarboxylic acid is a mixture of an aromatic dicarboxylic acid and at least one aliphatic dicarboxylic acid, that the diol is at least one aliphatic diol having the average carbon number of from 2.5 to 8.0, and that the polycondensation ester-based plasticizer is a polycondensation ester obtained from this dicarboxylic acid mixture and the diol.

The number-average molecular weight of the polycondensation ester-based plasticizer is preferably from 500 to 2,000, more preferably from 600 to 1,500, and still more preferably from 700 to 1,200. When the number average molecular weight of the polycondensation ester is 600 or greater, volatility becomes lower so that a film failure and process contamination due to sublimation under the high temperature condition during stretching of the resultant cellulose acylate film can be suppressed excellently.

Further, when the number average molecular weight of the polycondensation ester is 2,000 or less, compatibility with a cellulose acylate becomes higher so that the bleeding-out in film production and heat-stretching can be suppressed excellently.

In the case where a mixture of an aromatic dicarboxylic acid and an aliphatic dicarboxylic acid is used as the dicarboxylic acid component, the average carbon number of dicarboxylic acid components is preferably from 5.5 to 10.0, and more preferably from 5.6 to 8.0.

When the average carbon number of dicarboxylic acids is 5.5 or greater, a polarizing plate having excellent durability can be obtained. When the average carbon number of dicarboxylic acids is 10 or less, the compatibility with the cellulose acylate is excellent so that generation of the bleeding-out in film production process of the cellulose acylate film can be suppressed excellently.

Examples of the aromatic dicarboxylic acid, which is used for the synthesis of the polycondensation ester-based plasticizer, include phthalic acid, terephthalic acid, isophthalic acid, 1,5-naphthalenedicarboxylic acid, 1,4-naphthalenedicarboxylic acid, 1,8-naphthalenedicarboxylic acid, 2,8-naphthalenedicarboxylic acid, and 2,6-naphthalenedicarboxylic acid. Of these aromatic dicarboxylic acids, phthalic acid, terephthalic acid, and 2,6-naphthalenedicarboxylic acid are preferred; phthalic acid, and terephthalic acid are more preferred; and terephthalic acid is still more preferred.

The polycondensation ester obtained from a diol and a dicarboxylic acid including an aliphatic dicarboxylic acid, contains an aliphatic dicarboxylic acid residue.

Examples of the aliphatic dicarboxylic acid, which is used for synthesis of the polycondensation ester-based plasticizer, include oxalic acid, malonic acid, succinic acid, maleic acid, fumaric acid, glutaric acid, adipic acid, pimelic acid, suberic acid, azelaic acid, sebacic acid, dodecanedicarboxylic acid, and 1,4-cyclohexanedicarboxylic acid.

Examples of the diol, which synthesizes the polycondensation ester-based plasticizer, include an aromatic diol and an aliphatic diol, and in the present invention, it is preferable that the polycondensation ester is synthesized from at least an aliphatic diol.

The polycondensation ester-based plasticizer preferably contains an aliphatic diol residue having the average carbon number from 2.5 to 7.0, and more preferably contains an aliphatic diol residue having the average carbon number from 2.5 or 4.0.

When the average carbon number of aliphatic diol residues is 7.0 or less, compatibility with a cellulose acylate is improved, the bleeding-out, the increases of the loss on heating of the compound, and occurrence of surface state failure, which is considered to be caused by process contamination in drying process of a cellulose acylate web, can be suppressed excellently. In addition, when the average carbon number of aliphatic diol residues is 2.5 or greater, the synthesis is easy.

As the aliphatic diol used for synthesizing the polycondensation ester-based plasticizer, alkyl diols or alicyclic diols are preferred, for example, at least one of ethylene glycol, 1,2-propanediol, and 1,3-propanediol, and particularly preferably at least one of ethylene glycol, and 1,2-propanediol.

The terminal of the polycondensation ester-based plasticizer may be the diol or carboxylic acid as it is without being sealed (that is, the terminal of the polymer chain is —OH or CO₂H), or further sealing of the terminal may be conducted upon reaction with monocarboxylic acids or monoalcohols. When the terminal of the polycondensation ester-based plasticizer is sealed, it is possible to obtain an effect that the state at an ordinary temperature is hardly changed to a solid form, which results in good handling, and a cellulose acylate film having excellent humidity stability and capable of giving polarizer durability can be obtained.

As the polycondensation ester-based plasticizer, the compounds J-1 to J-38 described in JP-A-2012-234159, paragraphs [0062] to [0064] are also preferable.

In addition, a polycondensation ester-based plasticizer described below can also be preferably used in addition to those.

TABLE 1

| | Dicarboxylic acid | | | Diol | | | |
|---|---|---|---|---|---|---|---|
| | Aromatic | Aliphatic | Dicarboxylic acid | | | Diol | |
| | dicarboxylic acid | dicarboxylic acid | mol% ratio | Diol 1 | Diol 2 | mol% ratio | |
| No. | (dc1) | (dc2) | (dc1/dc2) | (do1) | (do2) | (do1/do2) | Terminal |
| J-40 | PA | AA | 25/75 | ED | — | 100/0 | Acetyl ester group |
| J-41 | PA | AA | 50/50 | ED | — | 100/0 | Acetyl ester group |
| J-42 | PA | — | 100/0 | ED | — | 100/0 | Acetyl ester group |
| J-43 | — | AA | 0/100 | ED | PD | 70/30 | Acetyl ester group |
| J-44 | — | AA | 0/100 | ED | PD | 50/50 | Acetyl ester group |

In Table 1, “PA” represents phthalic acid, “AA” represents adipic acid, “ED” represents ethanediol, and “PD” represents propanediol.

(Polyvalent Alcohol Ester-Based Plasticizer)

The polyhydric alcohol ester-based plasticizer used in the present invention is an ester derived from a polyhydric alcohol having two or more hydroxy groups at the alcohol moiety, and as the alcohol for the alcohol moiety, an alcohol is preferable, in which a saturated hydrocarbon which may be separated via an ether bond is substituted with two or more hydroxy groups, in addition to a hydroxy group.

A polyvalent alcohol that is a raw material for the polyvalent alcohol ester-based plasticizer is represented by formula (c). Formula (c)

Rα-(OH)m

In formula (c), Rα represents m-valent organic groups, and m represents a positive integer of 2 or greater.

The number of carbon atoms of the polyvalent alcohol is preferably 5 or more, and more preferably 5 to 20.

Examples of such a polyvalent alcohol include sugar alcohol, and glycols.

Specifically, triethyleneglycol, tetraethyleneglycol, dipropyleneglycol, tripropyleneglycol, sorbitol, trimethylol propane, and xylitol are preferred.

The acid moiety (the acyl moiety of the ester) of the polyhydric alcohol ester is preferably an acid moiety derived from a monocarboxylic acid. Examples of such an acid include an aliphatic monocarboxylic acid, an alicyclic monocarboxylic acid, and an aromatic monocarboxylic acid. When the alicyclic monocarboxylic acid or the aromatic monocarboxylic acid is used, it is preferable from the viewpoint of improving water-vapor transmission property and reservation property.

The number of carbon atoms of the aliphatic monocarboxylic acid is preferably 1 to 32, more preferably 1 to 20, and particularly preferably 1 to 10. It is preferable to contain acetic acid so as to increase the compatibility with the cellulose derivative, and it is also preferable to mix acetic acid with another monocarboxylic acid to be used.

Preferable examples of the aliphatic monocarboxylic acid include: saturated fatty acids, such as acetic acid, propionic acid, butyric acid, valeric acid, caproic acid, enanthic acid, caprylic acid, pelargonic acid, capric acid, 2-ethyl-hexane carboxylic acid, undecylic acid, lauric acid, tridecylic acid, myristic acid, pentadecylic acid, palmitic acid, heptadecylic acid, stearic acid, nonadecanoic acid, arachidic acid, behenic acid, rigniceric acid, cerotic acid, heptacosanic acid, montanic acid, melisic acid, and lacceric acid; and unsaturated fatty acids, such as undecylenic acid, oleic acid, sorbic acid, linolic acid, linolenic acid, and arachidonic acid.

Preferable examples of the alicyclic monocarboxylic acid include cyclopentane carboxylic acid, cyclohexane carboxylic acid, cyclooctane carboxylic acid, and their derivatives.

Preferable examples of the aromatic monocarboxylic acid include benzoic acid; those acids, such as toluic acid, in which an alkyl group is introduced into the benzene ring of benzoic acid; aromatic monocarboxylic acids having two or more benzene rings, such as biphenyl carboxylic acid, naphthalene carboxylic acid, and tetralin carboxylic acid; and their derivatives. Especially, benzoic acid is preferred.

Although the molecular weight of the polyvalent alcohol ester-based plasticizer is not particularly limited, the molecular weight is preferably from 300 to 3,000, and more preferably from 350 to 1,500. It is preferable to have a high molecular weight, from the viewpoint of excellent suppression of volatilization from the optical film, but it is preferable to have a low molecular weight, from the viewpoint of the water-vapor transmission property and the compatibility with a cellulose derivative.

The polyhydric alcohol ester-based plasticizer is preferably a compound described in, for example, paragraphs [0045] to [0049] of JP 2012-234159 A, which is preferably incorporated by reference.

### (Carbohydrate Derivative-Based Plasticizer)

As the carbohydrate derivative-based plasticizer, derivatives of carbohydrates including monosaccharides or from 2 to 10 monosaccharide units are preferred; and acylated ones of these derivatives are more preferred.

Preferred examples of the carbohydrates including monosaccharides or from 2 to 10 monosaccharide units include: ribose, arabinose, xylose, lyxose, glucose, fructose, mannose, galactose, trehalose, maltose, cellobiose, lactose, sucrose, sucralose, α-cyclodextrin, β-cyclodextrin, γ-cyclodextrin, δ-cyclodextrin, xylitol, and sorbitol. Further preferred examples thereof include: arabinose, xylose, glucose, fructose, mannose, galactose, maltose, cellobiose, sucrose, β-cyclodextrin, and γ-cyclodextrin; and particularly preferred examples thereof include: xylose, glucose, fructose, mannose, galactose, maltose, cellobiose, sucrose, xylitol, and sorbitol.

Preferred examples of the carbohydrate derivative-based plasticizer include maltose octaacetate, cellobiose octaacetate, sucrose octaacetate, xylose tetrapropionate, glucose pentapropionate, fructose pentapropionate, mannose pentapropionate, galactose pentapropionate, maltose octapropionate, cellobiose octapropionate, sucrose octapropionate, xylose tetrabenzoate, glucose pentabenzoate, fructose pentabenzoate, mannose pentabenzoate, galactose pentabenzoate, maltose octabenzoate, cellobiose octabenzoate, sucrose octabenzoate, xylitol pentabenzoate, and sorbitol hexabenzoate.

The carbohydrate derivative-based plasticizer preferably has a pyranose structure or a furanose structure.

As the carbohydrate derivative-based plasticizer, compounds described in JP-A-2012-234159, paragraphs [0030] to [0039] are preferable.

Meanwhile, in the present invention, the contents described in paragraphs to [0068] of JP 2012-234159 A are preferably adopted for the plasticizer, and the contents described in these paragraphs are preferably incorporated by reference.

The addition amount of the plasticizer is preferably from 1 to 20% by mass, with respect to 100 parts by mass of the cellulose acylate. When the content is 1% by mass or greater, an effect of improvement in polarizer durability can be easily achieved. While, on the other hand, when the content is 20% by mass or less, bleeding-out is suppressed. The content is more preferably from 2 to 15% by mass, and particularly preferably from 5 to 15% by mass. Meanwhile, two or more kinds of these plasticizers may be added. In the case of adding two or more kinds of those plasticizers, the specific example and the preferred range of the addition amount are the same as the above.

The timing of addition of the plasticizers to the cellulose acylate film is not particularly limited, as long as it is added at the time of film production. For example, it may be added at the time when the cellulose acylate is synthesized, or alternatively it may be mixed with the cellulose acylate at the time of preparing a dope.

### (Antioxidant)

The optical film of the present invention preferably contains an antioxidant. This antioxidant is possible to be added to a cellulose acylate solution. In the present invention, it is possible to add any antioxidant, for example, a phenol-based or hydroquinone-based antioxidant, such as 2,6-di-tert-butyl-4-methylphenol, 4,4′-thio-bis(6-tert-butyl-3-methylphenol), 1,1′-bis(4-hydroxyphenyecyclohexane, 2,2′-methylene-bis(4-ethyl-6-tert-butylphenol), 2,5-di-tert-butylhydroquinone, and pentaerythrityl-tetrakis[3-(3,5-di-tert-butyl-4-hydroxyphenyepropionate]. Further, it is preferable to add a phosphorus-based antioxidant, such as tris(4-methoxy-3,5-diphenyl)phosphite, tris(nonylphenyl)phosphite, tris(2,4-di-tert-butylphenyl)phosphite, bis(2,6-di-tert-butyl-4-methylphenyepentaerythritol diphosphite, and bis(2,4-di-tert-butylphenyl)pentaerythritol diphosphite.

As for the content of the antioxidant, the antioxidant is preferably added in the proportion of from 0.001 to 5.0 parts by mass, more preferably from 0.01 to 5.0 parts, with respect to 100 parts by mass of the cellulosic acylate.

### (Radical Scavenger)

The optical film of the present invention preferably contains a radical scavenger. A HALS (hindered amine-based light stabilizer) and a reductone are preferably used, as the radical scavenger.

The HALS is particularly preferably a compound having a 2,2,6,6-tetramethyl-piperidine ring, it is preferably a compound in which the 1-position of piperidine is a hydrogen atom, an alkyl group, an alkoxy group, a hydroxy group, an oxy radical group (—O—), an acyloxy group, or an acyl group, and it is more preferably a compound in which the 4-position thereof is a hydrogen atom, a hydroxy group, an acyloxy group, an amino group which may have a substituent, an alkoxy group, or an aryloxy group. In addition, it is also preferably a compound having from two to five 2,2,6,6-tetramethyl-piperidine rings in the molecule. Examples of such a compound include the compound described in paragraphs [0028] to [0052] of JP-A-2012-98698.

Examples of such a compound include Sunlizer HA-622 (trade name, manufactured by Sort K.K.); CHIMASSORB 2020FDL, TINUVIN 770DF, TINUVIN 152, TINUVIN 123, and FLAMESTAB NOR 116 FF (each trade name, manufactured by BASF Japan Ltd. (the former Chiba Specialty Chemicals)); CYASORB UV-3346, and CYASORB UV-3529 (each trade name, manufactured by SUN CHEMICAL Company Ltd.), and ADK STAB LA-72, and ADK STAB LA-81 (each trade name, manufactured by ADEKA Corporation).

Examples of the reductones include compounds exemplified in JP-A-6-27599, paragraphs [0014] to [0034]; compounds exemplified in JP-A-6-110163, paragraphs

to [0020]; and compounds exemplified in JP-A-8-114899, paragraphs [0022] to [0031].

In addition, it is possible to use preferably an oil-solubilized derivative of ascorbic acid or erythorbic acid, and examples thereof include L-ascorbyl stearate, L-ascorbyl tetraisopalmitate, L-ascorbyl palmitate, erythorbyl palmitate, and erythorbyl tetraisopalmitate. Among them, those having an ascorbic acid skeleton are preferable, and myristate, palmitate, and stearate of L-ascorbic acid are particularly preferable.

The content of the radical scavenger in the cellulose acylate film is preferably from 0.001 to 2.0 parts by mass, and more preferably from 0.01 to 1.0 parts by mass, with respect to 100 parts by mass of cellulose acylate.

### (Other Degradation Inhibitor)

As the degradation inhibitor of cellulose acylate, it is possible to use an additive, which is known as a peroxide decomposer, a radical inhibitor, or a metal deactivator. Examples of the additive include compounds described in JP-A-2006-251746, paragraphs [0074] to [0081] and [0082] to [0117].

The radical scavenger also exhibits the degradation preventing action, but an amine is also known as a degradation inhibitor. Examples thereof include compounds described in JP-A-5-194789, paragraphs [0009] to [0080]; and an aliphatic amine, such as tri-n-octylamine, triisooctylamine, tris(2-ethylhexyl)amine, and N,N-dimethyldodecylamine.

In addition, it is also preferable to use a polyvalent amine having two or more amino groups, and those having two or more primary or secondary amino groups are preferable, as the polyvalent amine. Examples of the compound having two or more amino groups include a nitrogen-containing heterocyclic compound (a compound having a pyrazolidine ring, a piperazine ring, or the like), and a polyamine-based compound (a compound which is a chain or cyclic polyamine and contains, for example, diethylenetriamine, tetraethylenepentamine, N,N′-bis(aminoethyl)-1,3-propanediamine, N,N,N′,N″,N″-pentakis(2-hydroxypropyl)diethylenetriamine, polyethyleneimine, modified polyethyleneimine, or cyclam as a basic skeleton).

Specific examples of such a polyvalent amine include diethylenetriamine, triethylenetetramine, tetraethylenepentamine, pentaethylenehexamine, dipropylenetriamine, tripropylenetetramine, aminoethylethanolamine, polyethyleneimine, ethylene oxide-modified polyethyleneimine, propylene oxide-modified polyethyleneimine, polyallylamine, polyvinylamine, N′,N′-tetrakis(2-hydroxyethyl)ethylenediamine, N,N,N′,N′-tetrakis(2-hydroxypropyl)ethylenediamine, and N,N,N′,N″,N″-pentakis(2-hydroxypropyl)diethylenetriamine. In addition, examples of the commercially available product include EPOMIN SP-006, SP-012, SP-018, and PP-061, manufactured by Nippon Shokubai Co., Ltd.

The content of the degradation inhibitor in the cellulose acylate film is preferably from 1 ppm to 10%, more preferably from 1 ppm to 5.0%, and still more preferably from 10 ppm to 1.0%, on the mass basis.

### (Ultraviolet Absorber)

The optical film of the present invention may contain an ultraviolet absorber, from the viewpoint of preventing deterioration of a resultant polarizing plate, a resultant liquid crystal, or the like. This ultraviolet absorber is possible to be added to a cellulose acylate solution. In the present invention, as the ultraviolet absorber, it is preferable to use those which have excellent absorption capacity of ultraviolet with wavelength 370 nm or less, and which exhibit a low absorption of visible light with wavelength 400 nm or longer, from the viewpoint of good property for the liquid crystal display. Examples of the ultraviolet absorber, which is preferably used in the present invention, include a hindered phenol-based compound, a hydroxybenzophenone-based compound, a benzotriazole-based compound, a salicylic acid ester-based compound, a benzophenone-based compound, a cyano acrylate-based compound, and a nickel complex-based compound.

The hindered phenol compound, although it is not particularly limited, is preferably at least one compound selected from the group consisting of 2,6-di-tert-butyl-p-cresol, pentaerythrityl-tetrakis[3-(3,5-di-tert-butyl-4-hydroxyphenyepropionate], N,N′-hexamethylene-bis(3,5-di-tert-butyl-4-hydroxy-hydrocinnamide), 1,3,5-trimethyl-2,4,6- tris(3,5-di-tert-butyl-4-hydroxybenzyl)benzene, and tris(3,5-di-tert-butyl-4-hydroxybenzyeisocyanurate.

The benzotriazole compound, although it is not particularly limited, is preferably at least one compound selected from the group consisting of 2-(2′-hydroxy-5′-methylphenyl)benzotriazole, 2,2-methylene-bis[4-(1,1,3,3-tetramethylbutyl)-6-(2H-benzotriazole-2-yl) phenol], (2,4-bis-(n-octylthio)-6-(4-hydroxy-3,5-di-tert-butylanilino)-1,3,5-triazine, triethyleneglycol-bis[3-(3-tert-butyl-5-methyl-4-hydroxyphenyl)propionate], N,N′-hexamethylene-bis(3,5-di-tert-butyl-4-hydroxy-hydrocinnamide), 1,3,5-trimethyl-2,4,6-tris(3,5-di-tert-butyl-4-hydroxybenzyl)benzene, 2-(2′-hydroxy-3′,5′-di-tert-butylphenyl)-5-chlorobenzotriazole, 2-(2′-hydroxy-3′,5′-di-tert-amylphenyl)-5-chlorobenzotriazole, 2,6-di-tert-butyl-p-cresol, pentaerythrityl-tetrakis[3-(3,5-di-tert-butyl-4-hydroxyphenyl) propionate], 2-(3,5-di-tert-amyl-2-hydroxyphenyl)benzotriazole, and 2-(2H-benzotriazole-2-yl)-6-(1-methyl-1-phenylethyl)-4-(1,1,3,3-tetramethylbutyl)phenol.

As these compounds, there is the Tinuvin series, such as Tinuvin 99-2, Tinuvin 109, Tinuvin 171, Tinuvin 320, Tinuvin 326, Tinuvin 327, Tinuvin 328, Tinuvin 329, Tinuvin 343, Tinuvin 900, Tinuvin 928, Tinuvin P, and Tinuvin PS, among the commercially available products, all of these are the products of BASF and can be preferably used.

The content of the ultraviolet absorber in the cellulose acylate film is preferably from 1 ppm to 10%, more preferably from 1 ppm to 5.0% and still more preferably from 10 ppm to 5.0%, on the mass basis.

### (Peeling Promoter)

Any peeling promoters may be added to the cellulose acylate film of the present invention.

The peeling promoter is preferably an organic acid, a polyvalent carboxylic acid derivative, a surfactant or a chelating agent. For example, compounds described in JP-A-2006-45497, paragraphs [0048] to [0081], compounds described in JP-A-2002-322294, paragraphs [0077] to [0086], and compounds described in JP-A-2012-72348, paragraphs [0030] to [0056], can be preferably used. The content of the peeling promoter in the cellulose acylate film is preferably from 1 ppm to 5.0%, more preferably from 1 ppm to 2.0%, on the mass basis.

Examples of the organic acid include compounds described in JP-A-2002-322294, paragraphs [0079] to [0082]. Specific examples thereof include citric acid, oxalic acid, adipic acid, succinic acid, malic acid, and tartaric acid.

As the organic acid, amino acids are also preferable. Examples thereof include asparagine, aspartic acid, adenine, alanine, β-alanine, arginine, isoleucine, glycin, glutamine, glutamic acid, serine, tyrosine, tryptophan, threonine, norleucine, valine, phenylalanine, methionine, lysine, and leucine.

The organic acid may be used as a free acid, and an alkali metal salt, an alkaline earth metal salt, and a salt of a heavy metal including a transition metal may be exemplified. Among the metals of the respective salts, examples of the alkali metal include lithium, potassium, and sodium, and examples of the alkaline earth metal include calcium, magnesium, barium, and strontium. Examples of the heavy metal including a transition metal include aluminum, zinc, tin, nickel, iron, lead, copper, and silver. In addition, a salt of a substituted or unsubstituted amine having 5 or less carbon atoms is also preferable, and examples of such a amine for a salt include ammonium, methylamine, ethylamine, propylamine, butylamine, dimethylamine, trimethylamine, triethylamine, hydroxyethylamine, bis(hydroxyethyl)amine, and tris(hydroxyethyl)amine The preferred metal is sodium among the alkali metals, and calcium and magnesium among the alkaline earth metals. Each of these alkali metals and alkaline earth metals can be used singly or in combination of two or more kinds thereof, and the alkali metals and the alkaline earth metals may be used in combination.

An ester compound and an amide compound are preferable, as the polyvalent carboxylic acid derivative.

The carboxylic acid component is a polyvalent carboxylic acid, and the carboxylic acid may be either an aliphatic carboxylic acid or an aromatic carboxylic acid but is preferably an aliphatic carboxylic acid. The aliphatic carboxylic acid may be saturated or unsaturated, may be a straight chain, branched chain, or cyclic aliphatic carboxylic acid, and may have a substituent. Examples of such a substituent include an alkyl group, an alkenyl group, an aryl group, a hydroxy group, an amino group, an alkoxy group, an alkenyloxy group, an acyloxy group, and an acylamino group.

Examples of the aromatic carboxylic acid include phthalic acid, terephthalicacid, isophthalicacid, and 1,3,5-benzene-tricarboxylic acid. Examples of the aliphatic carboxylic acid include oxalic acid, malonic acid, succinic acid, glutaric acid, adipic acid, and sebacic acid. Examples of the aliphatic carboxylic acid having a substituent include malic acid, citric acid, and tartaric acid.

In the polyvalent carboxylic acid ester, the group which is an alcohol component and is bonded to the oxygen atom of —C(═O)—O— of the ester functional group, is preferably a substituted or unsubstituted alkyl group [for example, methyl, ethyl, isopropyl, t-butyl, 2-ethylhexyl, and —CH₂CH₂O—(CH₂CH₂)n-C₂H₅], and an alkenyl group (for example, vinyl, allyl, 2-methyl-2-propenyl, 2-butenyl, and oleyl), and the total number of carbon atoms of the alcohol component (the group bonded to the oxygen atom) is preferably from 1 to 200, more preferably from 1 to 100, and still more preferably from 1 to 50. The substituent which the alkyl group or the alkenyl group may have, is preferably an alkoxy group, an alkenyloxy group, a hydroxy group, and an acyloxy group, and more preferably an alkoxy group. The alkoxy group or the alkenyloxy group is preferably those containing a (poly)oxyalkylene group, and particularly the (poly)oxyalkylene group is preferably a poly(oxyethylene) group, a (poly)oxypropylene group, and a (poly)oxybutylene group.

In addition, the raw material alcohol of the alcohol component may be monovalent or polyvalent, and examples of the polyhydric alcohol include ethylene glycol, propylene glycol, glycerin, and pentaerythritol, and those are also preferable in which the hydroxy group moiety (—OH) of these is a polyoxyalkyleneoxy group [for example, —(OCH₂CH₂)n-OH, —(OC₃H₆)ₙOH].

In the polyvalent carboxylic acid amide, the amine compound of the amine component may be either a primary or secondary, and is not particularly limited. The substituent to be substituted to the nitrogen atom of —C(═O)—N< of the amide functional group is preferably an alkyl group [for example, methyl, ethyl, isopropyl, t-butyl, 2-ethylhexyl, and —CH₂CH₂O—(CH₂CH₂)n-C₂H₅], and an alkenyl group (for example, vinyl, allyl, 2-methyl-2-propenyl, and 2-butenyl), and the total number of carbon atoms of the amine compound of the amine component is preferably from 1 to 200, more preferably from 1 to 100, and still more preferably from 1 to 50. The substituent which the alkyl group or the alkenyl group may have, is preferably an alkoxy group, an alkenyloxy group, a hydroxy group, an acyloxy group, an amino group, and an acylamino group, and more preferably an alkoxy group. The alkoxy group or the alkenyloxy group is preferably those containing a (poly)oxyalkylene group, and particularly the (poly)oxyalkylene group is preferably a polyoxyethylene) group, a (poly)oxypropylene group, and a (poly)oxybutylene group. In addition, it is also preferable that such a polyoxyalkylene moiety structure contains a branched polyoxyalkylene group, via glycerin.

In addition, the raw material amine compound of the amine component may be monovalent or polyvalent.

Among the polyvalent carboxylic acid derivatives, an organic acid monoglyceride having a carboxyl group which is unreacted and releasable is particularly preferable, and examples of the commercially available product thereof include Poem K-37V (glycerin citric acid/oleic acid ester) manufactured by Riken Vitamin Co., Ltd. and Step SS (glycerin stearyl acid/palmitic acid/succinic acid ester) manufactured by Kao Corporation.

As the surfactant, compounds described in JP-A-2006-45497, paragraphs [0050] to [0051], and compounds described in JP-A-2002-322294, paragraphs [0127] to [0128] are preferable used. Specific examples of the nonionic surfactant include a polyoxyethylene alkyl ether, a polyoxyethylene alkyl phenyl ether, polyoxyethylene/polyoxypropylene glycol, a fatty acid partial ester of polyhydric alcohol, a fatty acid partial ester of polyoxyethylene polyhydric alcohol, a polyoxyethylene fatty acid ester, a polyglycerin fatty acid ester, a fatty acid diethanolamide, a fatty acid partial ester of triethanolamine, and a polyether amine. In addition, examples of the commercially available product thereof include NYMEEN L-202, STAFOAM DO, and STAFOAM DL (NOF).

The chelating agent is a compound capable of coordinating (chelating) with a polyvalent metal ion, such as a metal ion, for example, an iron ion, or an alkaline earth metal ion, for example, a calcium ion. It is also possible to use any of various chelating agents, represented by amino(poly-carboxylic acid), amino(poly-phosphonic acid), alkylphosphonic acid, and phosphonocarboxylic acid. As the chelating agent, usable herein are compounds described in JP-B-6-8956 (“JP-B” means examined Japanese patent application), JP-A-11-190892, JP-A-2000-18038, JP-A-2010-158640, JP-A-2006-328203, JP-A-2005-68246, and JP-A-2006-306969.

Specific examples include ethylenediamine tetraacetic acid, hydroxyethylethylenediamine triacetic acid, diethylenetriamine pentaacetic acid, nitrilo triacetic acid, triethylenetetramine hexaacetic acid, cyclohexanediamine tetraacetic acid, hydroxyethylimino diacetic acid, ethylene glycol bis(2-aminoethyl ether) tetraacetic acid, 1,3-diaminopropane tetraacetic acid, phosphonic acid,l-hydroxyethylidene-1,1-diphosphonic acid, nitrilo-N,N,N-trimethylene phosphonic acid, ethylenediamine-N,N,N′,N′-tetramethylene phosphonic acid, ethylenediamine-di(o-hydroxyphenylacetic acid), DL-alanine-N,N-diacetic acid, aspartic acid-N,N′-diacetic acid, glutamic acid-N,N′-diacetic acid, serine-N,N′-diacetic acid, polyacrylic acid, an isoamylene/maleic acid copolymer, an acrylic acid/maleic acid copolymer, an acrylic acid/methacrylic acid copolymer, silicic acid, gluconic acid, hydroxybenzylimino diacetic acid, and imino diacetic acid. In addition, it is also preferable to use an oil-soluble chelating agent. It is possible to use Techrun DO (Nagase ChemteX Corporation), and CHELEST MZ-2 and CHELEST MZ-8 (Chelest Corporation) as a commercially available product.

### (Matting Agent)

A matting agent may be added to the optical film of the present invention, from the viewpoint of film lubricity (slipping property) and stable production. The matting agent may be either a matting agent composed of an inorganic compound or a matting agent composed of an organic compound.

Preferable examples of the matting agent composed of the inorganic compound include silicon-containing inorganic compounds (e.g., silicon dioxide, calcined calcium silicate, hydrated calcium silicate, aluminum silicate, magnesium silicate, etc.), titanium oxide, zinc oxide, aluminum oxide, barium oxide, zirconium oxide, strontium oxide, antimony oxide, tin oxide, tin/antimony oxide, calcium carbonate, talc, clay, calcined kaolin, calcium phosphate. Further, silicon-containing inorganic compounds and zirconium oxide are more preferred. Silicon dioxide is particularly preferred for use, because it is capable of reducing haze (turbidity) of the cellulose acylate film.

As fine particles of silicon dioxide, for example, commercial products which have trade names, such as Aerosil R972, R974, R812, 200, 300, R202, OX50, and TT600 (all by Nippon Aerosil), are usable. As fine particles of zirconium oxide, for example, commercial products which have trade names, such as Aerosil R976, and R811 (both by Nippon Aerosil), are usable.

Preferable examples of the matting agent composed of the organic compound include polymers, such as silicone resins, fluororesins, and acrylic resins. Above all, silicone resins are more preferred. Of the silicone resins, those having a three-dimensional network structure are particularly preferred. For example, it is possible to use commercially available products having trade names of Tospearl 103, Tospearl 105, Tospearl 108, Tospearl 120, Tospearl 145, Tospearl 3120, and Tospearl 240 (all manufactured by Toshiba Silicone Co., Ltd.).

When the matting agent is added to the cellulose acylate solution, the method is not particularly limited, and any method may be used with no problem, as long as a desired cellulose acylate solution can be obtained. For example, the additive may be added in the stage where a cellulose acylate is mixed with a solvent; or after preparing a mixture solution from a cellulose acylate and a solvent, the additive may be added thereto.

Further, a “just before” addition method, which the additive may be added to and mixed with a dope just before casting of the dope, may be used, and the mixing is conducted by screw kneading provided on-line. Specifically, a static mixer like an in-line mixer is preferred. As the in-line mixer, for example, a static mixer, SWJ (Toray's static intratubular mixer, Hi-Mixer, manufactured by Toray Engineering Co., Ltd.) is preferred.

Regarding the in-line addition, JP-A-2003-053752 describes a method of producing a cellulose acylate film, in which, for the purpose of preventing concentration unevenness and/or particle aggregation, the distance L between a nozzle tip, through which an additive liquid having a composition different from that of a main raw material dope is added, and a start end of the in-line mixer, is controlled to be at most 5 times the inner diameter d of the main raw material-feeding pipe, thereby preventing concentration unevenness, aggregation of matting particles, and the like. JP-A-2003-053752 discloses a more preferred embodiment, in which the distance (L) between the nozzle tip opening, through which the additive liquid having a composition different from that of the main raw material dope is added, and the start end of the in-line mixer is controlled to be at most 10 times the inner diameter (d) of the feeding nozzle tip opening, and the in-line mixer is a static non-stirring tubular mixer or a dynamic stirring tubular mixer. More specifically, JP-A-2003-053752 discloses that the flow rate of the cellulose acylate film main raw material dope/in-line additive liquid is from 10/1 to 500/1, and preferably from 50/1 to 200/1. JP-A-2003-014933 discloses a method of providing a phase difference film which is free from a trouble of additive bleeding-out and a trouble of interlayer peeling-off and which has good lubricity and excellent transparency; and regarding the method of adding additives to the film, the patent reference discloses that the additive may be added to a dissolving tank, or the additive or a solution or dispersion of the additive may be added to the dope during solution sending in the process of from the dissolving tank to a co-casting die, and further discloses that in the latter case, a mixing means, such as a static mixer, is provided therein, for the purpose of enhancing the mixing efficiency.

The matting agent is particularly preferably contained in the proportion of from 0.05 to 1.0% by mass, with respect to the film. By setting to such a value, the haze of the cellulose acylate film does not increase, and, in the case where it is practically used in the LCD, it is possible to contribute to the suppression of inconveniences, such as a decrease in contrast and the occurrence of bright spot, and it is also possible to realize scratch resistance (excoriation resistance) in addition to the above.

### <Physical Properties of Cellulose Acylate Film>

### (Hardness)

With regard to the surface hardness, the Knoop hardness by the Knoop method using a Knoop indenter is high and the pencil hardness is also favorable. The Knoop hardness can be measured by a hardness tester with a Knoop indenter as the indenter, for example, the “FISCHERSCOPE H100Vp-type hardness tester” manufactured by Fischer Instruments.

The pencil hardness can be evaluated, for example, by the pencil hardness evaluation method regulated in JIS-K5400, using a test pencil regulated in JIS-S6006.

The compound represented by formula (I) according to the present invention is able to increase the hardness, such as the Knoop hardness of the cellulose acylate film, and the hardness can be adjusted by the kind or content of the compound represented by formula (I).

### (Elastic Modulus (Tensile Elastic Modulus))

The cellulose acylate film of the present invention exhibits practically-sufficient elastic modulus (tensile elastic modulus). The range of the elastic modulus, although it is not particularly limited, is preferably from 1.0 GPa to 7.0 GPa, and more preferably from 2.0 GPa to 6.5 GPa, from the viewpoint of production suitability and handling property. The compound represented by formula (I) according to the present invention acts such that the cellulose acylate film is hydrophobized by addition of the compound to a cellulose acylate, thereby improving elastic modulus. In this point, the present invention also has an advantage.

### (Photoelastic Coefficient)

The absolute value of photoelastic coefficient of the cellulose acylate film of the present invention is preferably 8.0×10⁻¹² m²/N or less, more preferably 6.0×10⁻¹² m²/N or less, and still more preferably 5.0×10⁻¹² m²/N or less. Lessening the photoelastic coefficient of the cellulose acylate film enables suppression of generation of unevenness under the conditions of high temperature and high humidity, upon mounting of the optical film of the present invention containing the cellulose acylate film into a liquid crystal display as a polarizing plate protective film. The photoelastic coefficient is measured and calculated in accordance with the following method, unless otherwise specified.

The lower limit of the photoelastic coefficient is not particularly limited, but it is practical to be 0.1×10⁻¹² m²/N or more.

A cellulose acylate film is cut into a specimen of 3.5 cm×12 cm, Re is measured under each load of non-load, 250 g, 500 g, 1,000 g and 1,500 g, using an ellipsometer (M 150 [trade name], manufactured by JASCO Corporation), and by the slope of a straight line of Re change to stress, the photoelastic coefficient is calculated.

### (Moisture Content)

The moisture content of the cellulose acylate film can be evaluated by measurement of equilibrium moisture content under the constant temperature and humidity. The equilibrium moisture content is obtained by the following method. That is, the moisture content of a sample, which has reached equilibrium after leaving it for 24 hours at the above temperature and humidity, is measured according to the Karl-Fischer method, and the thus-obtained moisture content (g) is divided by the sample mass (g), to obtain the equilibrium moisture content.

The moisture content of the cellulose acylate film of the present invention under the conditions of 25° C. and relative humidity of 80% is preferably 5% by mass or less, more preferably 4% by mass or less, and still more preferably less than 3% by mass. Lessening the moisture content of the cellulose acylate film enables suppression of generation of unevenness of the resultant display of a liquid crystal display, under the conditions of high temperature and high humidity, upon mounting of the optical film of the present invention containing the cellulose acylate film into the liquid crystal display as a polarizing plate protective film. The lower limit of the moisture content is not particularly limited, but it is practical to be 0.1% by mass or greater.

### (Water-Vapor Transmission Ratio)

The water-vapor transmission ratio of the cellulose acylate film can be measured and evaluated by the following method. That is, the mass of water-vapor, which passes through the sample for 24 hours in the atmosphere of temperature 40° C. and relative humidity 90%, is measured according to the water-vapor transmission ratio test (cup method) prescribed in JIS Z0208, and the thus-obtained value is converted to a mass of water-vapor passing through for 24 hours per m² of the sample area, to evaluate the water-vapor transmission ratio.

The water-vapor transmission ratio of the cellulose acylate film of the present invention is preferably from 500 to 2,000 g/m² ·day, and more preferably from 900 to 1,300 g/m²·day.

### (Haze)

The cellulose acylate film has a haze of preferably 1% or less, more preferably 0.7% or less, particularly preferably 0.5% or less. When the haze is lowered to the upper limit or less, the cellulose acylate film has advantages in that transparency of the film is more increased and thus the film becomes more usable as an optical film. The haze is measured according to the method below, unless otherwise specified. The lower limit of the haze is not particularly limited, but it is practical to be 0.001% by mass or greater.

With respect to the cellulose acylate film, the haze of the film specimens of 40 mm×80 mm in size is measured in an environment at 25° C. and 60% relative humidity, using a haze meter (HGM-2DP, Suga Test Instruments Co., Ltd.), in compliance with JIS K-7136.

### (Film Thickness)

The average film thickness of the cellulose acylate film is preferably from 10 to 100 μm, more preferably from 15 to 80 μm, and still more preferably from 15 to 70 μm. Setting the average film thickness to 20 μm or greater is preferable, because handling preferably in the production of a web film is improved. While, on the other hand, when the average film thickness is set to 70 μm or less, the response to humidity change becomes easy, and thus maintenance of the optical characteristics becomes easy.

Further, in the case where the cellulose acylate film has a multi-layered structure of three or more multi-layers, the film thickness of the core layer is preferably from 3 to 70 μm, and more preferably from 5 to 60 μm, and each of the film thicknesses of the skin layer A and skin layer B are more preferably from 0.5 to 20 μm, particularly preferably from 0.5 to 10 μm, and most preferably from 0.5 to 3 μm.

### (Width)

The film width of the cellulose acylate film is preferably from 700 to 3,000 mm, more preferably from 1,000 to 2,800 mm, and particularly preferably from 1,300 to 2,500 mm.

### <Production Method of Cellulose Acylate Film>

The production method of the cellulose acylate film of the present invention is not particularly limited, but the cellulose acylate film is preferably produced by a melt-casting method or a solvent-casting method. The production by a solvent-casting method is more preferable. Examples of production of a cellulose acylate film using a solvent-casting method are given in U.S. Pat. No. 2,336,310, U.S. Pat. No. 2,367,603, U.S. Pat. No. 2,492,078, U.S. Pat. No. 2,492,977, U.S. Pat. No. 2,492,978, U.S. Pat. No. 2,607,704, U.S. Pat. No. 2,739,069, U.S. Pat. No. 2,739,070, British Patent No. 640731, British Patent No. 736892, JP-B-45-4554, JP-B-49-5614, JP-A-60-176834, JP-A-60-203430, and JP-A-62-115035, and referred to herein. The cellulose acylate film may be stretched. Regarding the method and condition for stretching, for example, referred to are JP-A-62-115035, JP-A-4-152125, JP-A-4-284211, JP-A-4-298310, and JP-A-11-48271.

### (Casting Methods)

The casting methods for solutions include: a method for uniformly extruding a prepared dope from a pressure die onto a metal support; a doctor blade method for adjusting, with a blade, the film thickness of a dope once cast on a metal support; and a reverse roll coater method for adjusting it with a reverse rotating roll, but the method of using a pressure die is preferred. The pressure die includes a coat hanger type or a T-die type, and any of them may be preferably used. In addition to these methods exemplified herein, various methods of film production by casting a cellulose acylate solution, which are conventionally known in the art, may be employed. When each of conditions is set in consideration of the difference in the boiling points of solvents to be used, the same effects as the contents described in each publication can be obtained.

### Co-casting

In formation of the cellulose acylate film, a multi-layer casting method, such as a co-casting method, a sequential casting method and a coating method, is preferable. Especially, a simultaneous co-casting method is particularly preferred, from the viewpoints of stable production and reducing the production cost.

In the case where the film is produced according to a co-casting method or a sequential casting method, first, a cellulose acetate solution (dope) for each layer is prepared. The co-casting method (multilayer simultaneous casting method) is a casting method, in which individual layers are simultaneously cast by simultaneously extruding co-casting dopes onto a casting support (a band or a dram) from a casting Giesser through which the individual casting dopes for intended layers (the number of layers may be three or more) are simultaneously extruded via different slits and the like, and then at a suitable timing, the thus-formed film on the support is peeled-off and dried. In FIG. 3, the cross-sectional view shows a state in which casting is performed by simultaneously extruding three layers formed of dopes 1 for two surface layers and a dope 2 for a core layer on a casting support 4, using a co-casting Giesser 3.

The sequential casting method is a casting method, in which first a casting dope for first layer is extruded out and cast onto a casting support through a casting Giesser, then after it is dried or not dried, a casting dope for second layer is extruded through the casting Giesser and cast onto it, and if needed, three or more layers are sequentially formed by casting and laminating dopes in the same manner as the above, and then at a suitable timing, the resulting laminate is peeled-off from the support and dried, to form a cellulose acylate film. The coating method is generally a method, in which a core layer is formed in film-state by means of the solvent-casting method, then a coating solution for a surface layer is prepared, and then using a suitable coater, the coating solution is applied onto the core layer first on one surface thereof and next on the other surface thereof, or alternatively simultaneously on both surfaces thereof, and dried, to form a multi-layered cellulose acylate film.

As the endlessly running metal support for use in production of the cellulose acylate film, it is possible to use a dram the surface of which is mirror-finished by chromium plating, or a stainless belt (may be called as a band) the surface of which is mirror-finished by surface polish. One or at least two pressure dies may be used by arranging it or those, above the metal support. Preferably, one or two pressure dies are arranged. In the case where two or more pressure dies are arranged, a casting amount of the dope may be divided into moieties, which are suitable for the individual dies; or the casting dope may be fed to the die at a suitable proportion from a plurality of precision metering gear pumps. The temperature of the dope (resin solution) for use in casting is preferably from −10° C. to 55° C., and more preferably from 25° C. to 50° C. In this case, the solution temperature may be the same throughout the entire process, or may be different in different stages of the process. In the case where the temperatures are different in different stages, it is no problem as long as the dope has a desired temperature just before casting.

Further, the material of the above metal support, although it is not particularly limited, is preferably made of SUS (for example, SUS 316).

### (Peeling)

The method of producing the cellulose acylate film preferably includes a process of peeling-off the above dope film from the metal support. In the production method of the cellulose acylate film, the method of peeling-off it is not particularly limited, and the peeling property can be improved by any of methods known for peeling-off.

### (Stretching Process)

The method of producing the cellulose acylate film preferably includes a stretching process after film production. The stretching direction of the cellulose acylate film is preferable in any of a cellulose acylate film conveying direction (MD direction) and an orthogonal direction (TD direction) to the conveying direction, but the orthogonal direction (TD direction) to the cellulose acylate film conveying direction is particularly preferred, from the viewpoint of the subsequent polarizing plate-manufacturing process using the cellulose acylate film.

A method of stretching the film in the TD direction is described in, for example, JP-A-62-115035, JP-A-4-152125, JP-A-4-284211, JP-A-4-298310, JP-A-11-48271. In the case of stretching in the MD direction, the cellulose acylate film is stretched when the cellulose acylate film winding speed is set to be faster than the cellulose acylate film peeling-off speed, for example, by adjusting a speed of the cellulose acylate film-conveying roller. In the case of stretching in the TD direction, the cellulose acylate film may be stretched by conveying the cellulose acylate film while holding the width of the cellulose acylate film with a tenter, and extending the width of the tenter gradually. After drying the cellulose acylate film, the film may be also stretched by using a stretching machine (preferably uniaxial stretching by using a long stretching machine).

In the case where the cellulose acylate film is used as a protective film for a polarizer, the transmission axis of the polarizer and the in-plane slow axis of the cellulose acylate film are required to be arranged parallel to one another, in order to suppress the light leakage when viewed from oblique directions to the polarizing plate. The transmission axis of the roll film-shaped polarizer that is produced continuously is generally parallel to the width direction of the roll film. Therefore, in order to continuously sticking the above roll film-shaped polarizing element together with a protective film composed of the roll film-shaped cellulose acylate film, the in-plane slow axis of the roll film-shaped protective film is required to be parallel to the width direction of the cellulose acylate film. Accordingly, the film is preferably stretched to a larger extent in the TD direction. The stretching treatment may be conducted in the mid course of the film production process, or the original film obtained by rewinding the produced film may be subjected to a stretching treatment.

The stretching in the TD direction is preferably from 5 to 100%, more preferably from 5 to 80%, and particularly preferably from 5 to 40%. Meanwhile, non-stretching means that stretching is 0%. The stretching treatment may be conducted in the mid course of the film production process, or the original film obtained by rewinding the produced film may be subjected to a stretching treatment. In the former case, stretching may be conducted in the condition where a certain amount of a residual solvent is contained, and when the residual solvent amount, i.e. {(mass of residual volatile substance/mass of film after heat treatment)×100} (%), is from 0.05% to 50%, the stretching is preferably conducted. It is particularly preferable to conduct the stretching of from 5% to 80% in the condition where the residual solvent amount is from 0.05% to 5%.

### (Drying)

The method of producing the cellulose acylate film preferably includes: a step of drying the cellulose acylate film; and a step of stretching the thus-dried cellulose acylate film at a temperature, which is equal to or higher than {the glass transition temperature (Tg) −10° C.}, from the viewpoint of enhancing the retardation.

Drying of the dope provided on a metal support that is included in the production of the cellulose acylate film, generally includes: a method of blowing a hot air from a surface side of the metal support (a dram or a belt), that is to say, from the surface of a web provided on the metal support; a method of blowing a hot air from a back side of the dram or belt; a back-side liquid heat transfer method, in which a temperature-modulated liquid is brought into contact with the back side opposite to the casting side of the dram or belt, thereby heating the dram or belt through heat transfer to control a surface temperature; and the like. Among these, the back-side liquid heat transfer method is preferred. The surface temperature of the metal support before casting is conducted, is not particularly limited as long as it is not higher than the boiling point of a solvent, which is used for a dope. However, in order to promote drying or to make the dope lose fluidity on the metal support, the surface temperature is preferably set to a temperature, which is lower by 1° C. to 10° C. than the boiling point of the solvent having the lowest boiling point among the solvents used for the dope. However, this shall not apply in the case where the casting dope is cooled and then peeled-off without drying.

The adjustment of the cellulose acylate film thickness may be achieved by adjusting a concentration of the solid contained in the dope, a slit gap of the die nozzle, an extrusion pressure from a die, a speed of the metal support, or the like, so as to be a desired thickness.

The thus-obtained cellulose acylate film is preferably wound at the degree of from 100 to 10,000 m, more preferably from 500 to 7,000 m, and still more preferably from 1,000 to 6,000 m, in length per roll. At the time of winding, at least one end thereof is preferably subjected to knurling. The width of knurling is preferably from 3 mm to 50 mm and more preferably from 5 mm to 30 mm. The height thereof is preferably from 0.5 μm to 500 μm and more preferably from 1 μm to 200 μm. This may be either one-way press or two-way press.

When the optical film of the present invention is used as an optical compensation film for a large screen liquid crystal display, molding the film so as to be, for example, 1,470 mm or more in width, is preferred. Further, the aspect of the polarizing plate protective film of the present invention includes a film piece that is cut to a size capable of being mounted as it is in a liquid crystal display, as well as a film that is manufactured in a long shape by continuous production and wound in a roll shape. The polarizing plate protective film of the latter aspect is stored or conveyed as it is, and is used by cutting it to a desired size when the film is mounted in a liquid crystal display, or when the film and a polarizer or the like are stuck together in practice. Alternatively, the polarizing plate protective film is used by cutting it to a desired size when the film is mounted in a liquid crystal display in practice after sticking the film in a long shape as it is with a polarizer composed of a polyvinyl alcohol film or the like manufactured similarly in a long shape. As an aspect of the optical compensation film, which is wound in a roll shape, an aspect of a film, which is wound in a roll shape and has a roll length of 2,500 m or more, is exemplified.

### <<Hard Coat Layer>>

In the optical film of the present invention, the hard coat layer, which is provided on the cellulose acylate film if desired, is a layer for imparting hardness or scratch resistance to the optical film of the present invention. It is possible to form a hard coat layer exhibiting high adhesive property with respect to the cellulose acylate film in cooperation with the compound represented by formula (I), for example, by applying a coating composition on the cellulose acylate film and curing it. A filler and/or an additive may be added to the hard coat layer, thereby to make the hard coat layer itself have mechanical, electrical or optical physical performance or chemical performance, such as water repellency or oil repellency. The thickness of the hard coat layer is preferably 0.1 to 6 μm, more preferably from 3 to 6 μm. Having such a thin hard coat layer of which the thickness falls within the range, the resultant optical film containing the hard coat layer can have improved physical preferably in point of brittleness reduction and curling prevention, and can attain other advantages of weight saving and production cost cutting. Preferably, the hard coat layer is formed by curing a curable composition.

Preferably, the curable composition is prepared as a liquid coating composition. One example of the coating composition contains a monomer or an oligomer for a matrix formation binder, a polymer and an organic solvent. Curing the coating composition applied to the substrate film can form the intended hard coat layer. The curing reaction includes crosslinking or polymerization.

### (Monomer or Oligomer for Matrix Formation Binder)

Examples of the monomer or oligomer for the matrix formation binder usable in the present invention include ionizing radiation-curable polyfunctional monomers and polyfunctional oligomers. The polyfunctional monomers and the polyfunctional oligomers are preferably crosslinkable or polymerizable. The functional group in the ionizing radiation-curable polyfunctional monomers and polyfunctional oligomers is preferably polymerizable through exposure to light, electron beam or radiation; and above all, especially preferred is a photopolymerizing functional group.

Examples of the photopolymerizing functional group includes an unsaturated polymerizing functional group, such as a (meth)acryloyl group, a vinyl group, a styryl group, and an allyl group; and a ring-opening polymerizing functional group, such as those in epoxy compounds. Above all, preferred is a (meth)acryloyl group.

Specific examples of the photopolymerizing polyfunctional monomer having a photopolymerizing functional group include: (meth)acrylic diesters of alkylene glycols, such as neopentyl glycol acrylate, 1,6-hexanediol (meth)acrylate, and propylene glycol di(meth)acrylate; (meth)acrylic diesters of polyoxyalkylene glycols, such as triethylene glycol di(meth)acrylate, dipropylene glycol di(meth)acrylate, polyethylene glycol di(meth)acrylate, and polypropylene glycol di(meth)acrylate; (meth)acrylic diesters of polyvalent alcohols, such as pentaerythritol di(meth)acrylate; and (meth)acrylic diesters of ethylene oxide- or propylene oxide-adducts, such as 2,2-bis {4-(acryloxy.diethoxy)phenyl}propane, and 2,2-bis{4-(acryloxy.polypropoxy)phenyl}propane.

Further, urethane(eth)acrylates, polyester(meth)acrylates, isocyanuric acrylates, and epoxy(meth)acrylates are also preferred for use herein as the photopolymerizing polyfunctional monomer.

Of the above, more preferred are esters of polyvalent alcohols and (meth)acrylic acids, and even more preferred are polyfunctional monomers having at least three (meth)acryloyl groups in the molecule.

Specific examples thereof include (di)pentaerythritol tri(meth)acrylate, (di)pentaerythritol tetra(meth)acrylate, (di)pentaerythritol penta(meth)acrylate, (di)pentaerythritol hexa(meth)acrylate, tripentaerythritol triacrylate, tripentaerythritol hexaacrylate, trimethylolpropane tri(meth)acrylate, trimethylolethane tri(meth)acrylate, ditrimethylolpropane tetra(meth)acrylate, EO-modified trimethylolpropane tri(meth)acrylate, PO-modified trimethylolpropane tri(meth)acrylate, EO-modified phosphoric acid tri(meth)acrylate, 1,2,4-cyclohexane tetra(meth)acrylate, pentaglycerol triacrylate, 1,2,3-cyclohexane tetramethacrylate, polyester polyacrylate, and caprolactone-modified tris(acryloxyethyl)isocyanurate.

In this description, “(meth)acrylate”, “(meth)acrylic acid” and “(meth)acryloyl” each mean “acrylate or methacrylate”, “acrylic acid or methacrylic acid” and “acryloyl or methacryloyl”, respectively.

Further, oligomers or pre-polymers of polyfunctional compounds can be exemplified, such as resins having at least three (meth)acryloyl groups, for example, of polyester resins having a relatively low molecular weight, as well as polyether resins, acrylic resins, epoxy resins, urethane resins, alkyd resins, spiroacetal resins, polybutadiene resins, polythiol-polyene resins, and polyvalent alcohols.

As specific compounds of the polyfunctional acrylate-based compounds having at least three (meth)acryloyl groups, referred to is the description in JP-A-2007-256844, paragraph [0096], and the like.

As urethane (meth)acrylates, for example, there may be mentioned urethane (meth)acrylate-based compounds obtained by reacting a hydroxy group-containing compound, such as alcohol, polyol, and/or hydroxy group-containing (meth)acrylate, with an isocyanate, followed by optionally esterifying the polyurethane compound obtained through the reaction with (meth)acrylic acid.

As specific examples of those compounds, referred to is the description in JP-A-2007-256844, paragraph [0017], and the like.

Use of isocyanuric (meth)acrylates is preferred as reducing the curling of the formed film. Examples of the isocyanuric (meth)acrylates include isocyanuric diacrylates, and isocyanuric triacrylates; and as examples of those compounds, referred to is the description in JP-A-2007-256844, paragraphs [0018] to [0021], and the like.

An epoxy-based compound may further be used in the hard coat layer for reducing the shrinkage of the layer through curing. As the epoxy group-having monomers, usable are monomers having at least two epoxy groups in the molecule. Examples of the monomer include epoxy-based monomers described in JP-A-2004-264563, JP-A-2004-264564, JP-A-2005-37737, JP-A-2005-37738, JP-A-2005-140862, JP-A-2005-140863, JP-A-2002-322430, and the like. Also preferred is use of compounds having both epoxy-based and acrylic-based functional groups, such as glycidyl (meth)acrylate.

### (Polymer Compound)

The hard coat layer may contain a polymer compound. Adding a polymer compound to the layer is preferred, as capable of reducing the curing shrinkage of the layer and capable of facilitating the viscosity control of the coating liquid that takes an interest in the dispersion stability (coagulability) of resin particles. Other advantages of the polymer compound are that the polarity of the solidified matter in the drying step may be controlled to change the coagulation behavior of resin particles, and that the drying unevenness in the drying step can be reduced.

The polymer compound is already in the form of a polymer when it is added to the coating liquid. As the polymer compound of the type, preferred for use herein are, for example, cellulose esters (e.g. cellulose triacetate, cellulose diacetate, cellulose propionate, cellulose acetate propionate, cellulose acetate butyrate, and cellulose nitrate); and resins, such as urethanes, polyesters, (meth)acrylates (e.g. methyl methacrylate/methyl(meth)acrylate copolymer, methyl methacrylate/ethyl(meth)acrylate copolymer, methyl methacrylate/butyl(meth)acrylate copolymer, methyl methacrylate/styrene copolymer, methyl methacrylate/(meth) acrylic acid copolymer, and poly(methyl methacrylate)), and polystyrenes.

### (Curable Composition)

One example of the curable composition usable for forming the hard coat layer is a curable composition containing a (meth)acrylate-based compound. Preferably, the curable composition contains a photoradical polymerization initiator or a thermal radical polymerization initiator along with the (meth)acrylate-based compound, and if desired, may further contain a filler, a coating aid, and other additives. The curable composition may be cured through polymerization to be attained by exposure to ionizing radiation or to heat, in the presence of a photoradical polymerization initiator or a thermal radical polymerization initiator. Ionizing radiation curing and thermal curing may be combined. As the optical and thermal polymerization initiators, usable are commercial products, which are described in, for example, “Newest UV Curing Technology” (p. 159, issued by Kazuhiro Takausu, published by Technical Information Society of Japan, 1991), and Ciba Specialty Chemicals' catalogues.

Another example of the curable composition that can be used in forming the hard coat layer is a curable composition containing an epoxy-based compound. Preferably, the curable composition of the type contains an optical acid generator capable of generating a cation by the action of light applied thereto, along with the epoxy-based compound therein, and may optionally contain a filler, a coating aid, and other additives. The curable composition may be cured through polymerization to be attained by exposure to light, in the presence of the optical acid generator. Examples of the optical acid generator include ionic compounds, such as triarylsulfonium salts, and diaryliodonium salts; and nonionic compounds, such as sulfonic acid nitrobenzyl ester. Various types of any optical acid generators, such as the compounds described in “Imaging Organic Material” (edited by Organic Electronics Material Society of Japan, published by Bunshin Publishing, 1997), and the like.

A (meth)acrylate-based compound and an epoxy-based compound may be combined for use herein. In such a case, preferably, a photoradical polymerization initiator or a thermal polymerization initiator is combined with an optical cationic polymerization initiator.

The curable composition, which is particularly suitable for the formation of the hard coat layer, is a composition containing a (meth)acrylate-based compound, as used in Examples to be described below.

The curable composition is preferably prepared as a coating liquid. The coating liquid can be prepared, by dissolving and/or dispersing the above-mentioned ingredients in an organic solvent.

### (Property of Hard Coat Layer)

The hard coat layer formed on the cellulose acylate film of the optical film of the present invention exhibits high adhesive property with respect to the cellulose acylate film. In particular, the hard coat layer, which is formed on the cellulose acylate film containing the compound represented by formula (I) and which is composed of the suitable curable composition described above, is formed so as to exhibit higher adhesive property with respect to the cellulose acylate film, in cooperation of the curable composition with the compound represented by formula (I). Thus, the optical film of the present invention having such a cellulose acylate film and a hard coat layer is excellent in light resistance, since the adhesive property between the cellulose acylate film and the hard coat layer is maintained even when being irradiated with light or the like.

It is preferable that the hard coat layer is excellent in abrasion resistance. Concretely, when the layer is tested in a pencil hardness test (JIS-S6006) that is an index of abrasion resistance, the layer attains at least 3H.

### <<Polarizing Plate>>

The polarizing plate of the present invention includes at least a polarizer and the optical film of the present invention. The polarizing plate of the present invention preferably includes a polarizer and the optical film of the present invention provided on one side or both sides of the polarizer. Examples of the polarizer include an iodine-based polarizer, a dye-based polarizer using a dichroic dye, and a polyene-based polarizer. Ordinarily the iodine-based polarizer and the dye-based polarizer may be produced using a polyvinyl alcohol-based film. When the optical film of the present invention is used as a polarizing plate protective film, the production method of the polarizing plate is not particularly limited, and the polarizing plate may be produced according to a usual manner. For example, there is a method of subjecting the cellulose acylate film of the optical film of the present invention to an alkali treatment, besides preparing a polarizer by immersing a polyvinyl alcohol film in an iodine solution and stretching the film, and then sticking the thus-treated cellulose acylate film and both sides of the polarizer together with a completely-saponified polyvinyl alcohol aqueous solution. In place of the alkali treatment, an easy adhesion processing, as described in JP-A-6-94915 and JP-A-6-118232, may be used. Examples of the adhesive that is used for sticking the processed surface of the cellulose acylate film and both sides of polarize together, include polyvinyl alcohol-based adhesives, such as polyvinyl alcohol, and polyvinyl butyral; and vinyl-based latexes derived from butyl acrylate or the like.

The optical film of the present invention and the polarizer are preferably stuck together such that a transmission axis of the polarizer and a slow axis of the optical film of the present invention are substantially bisected at right angles. In the liquid crystal display of the present invention, it is preferable that the transmission axis of the polarizer and the slow axis of the optical film of the present invention are stuck together so as to be substantially bisected at right angles, be parallel, or 45° to each other.

Herein, to be substantially bisected at right angles or to be parallel includes the error range acceptable in the art to which the present invention belongs. For example, it means that it is within a range of less than ±10° from the exact angle to be parallel or to be bisected at right angles, and the difference from the exact angle is preferably 5° or less and more preferably 3° or less.

That the transmission axis of the polarizer and the slow axis of the cellulose acylate film are parallel to each other, means that the angle formed between the direction of the principal refractive index nx of the cellulose acylate film and the direction of the transmission axis of the polarizer is within a range of ±10°. The range of this angle is preferably ±5°, more preferably ±3°, even more preferably ±1°, and most preferably ±0.5°. Meanwhile, when this angle is 0°, the direction of the principal refractive index nx of the cellulose acylate film and the direction of the transmission axis of the polarizer do not intersect, but they are completely parallel to each other.

Further, that the transmission axis of the polarizer and the slow axis of the cellulose acylate film are bisected at right angles, means that the direction of principal refractive index nx of the cellulose acylate film and the direction of the transmission axis of the polarizer are crossed at the angle of 90°±10°. This angle, in which they are crossed, is preferably 90°±5°, more preferably 90°±3°, further preferably 90°±1°, and most preferably 90°±0.5°.

Setting the angle to the above range in sticking them together, enables further reduction in light leakage under the condition of polarizing plate cross nicol. The measurement of the slow axis can be performed by any various methods, and can be performed, for example, using a birefringence meter (KOBRA DH, manufactured by Oji Scientific Instruments).

The aspect of the polarizing plate of the present invention includes a film piece that is cut to a size capable of being mounted as it is in a liquid crystal display, as well as a film that is manufactured in a long shape by continuous production and wound in a roll shape (for example, an aspect having the roll length of 2,500 m or longer and an aspect having the roll length of 3,900 m or longer). When it is intended for the large-screen liquid crystal display, the width of the polarizing plate is preferably set to 1,470 mm or longer. The specific configuration of the polarizing plate of the present invention is not particularly limited, and any configuration may be used. For example, the configuration shown in FIG. 6 of JP-A-2008-262161 may be used.

### <<Liquid Crystal Display>>

The liquid crystal display of the present invention includes at least a liquid crystal cell and the polarizing plate of the present invention. In the liquid crystal display of the present invention, in the case where the liquid crystal display has the polarizing plate, e.g. a first polarizing plate and a second polarizing plate to be described below, the liquid crystal display is preferably an IPS, OCB, or VA mode in which at least either of the first or second polarizing plate is the polarizing plate of the present invention.

The liquid crystal display of the present invention preferably has a liquid crystal cell, and a polarizing plate, which is layered on both sides of the liquid crystal cell and equipped with an optical film on the surface of the side opposite to the liquid crystal cell side. In other words, it is preferable that the liquid crystal display of the present invention has the first polarizing plate, the liquid crystal cell, and the second polarizing plate, and that it is equipped with the optical film of the present invention on the surface opposite to the polarizing plate surface sandwiched between each of the polarizing plates and the liquid crystal cell. The liquid crystal display having such a configuration is excellent in the suppression of display unevenness and exerts high display performance.

In addition, the liquid crystal display of the present invention preferably has an optical film, particularly a cellulose acylate film, in which the polarizing plate disposed on the visual recognition side has a hard coat layer on the surface of the optical film on the visual recognition side. The liquid crystal display having such a configuration exerts excellent scratch resistance (excoriation resistance) and light resistance, in addition to high display performance excellent in the suppression of display unevenness.

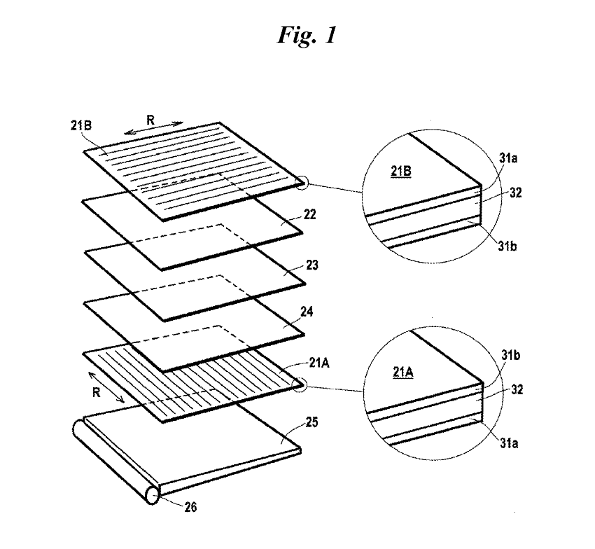

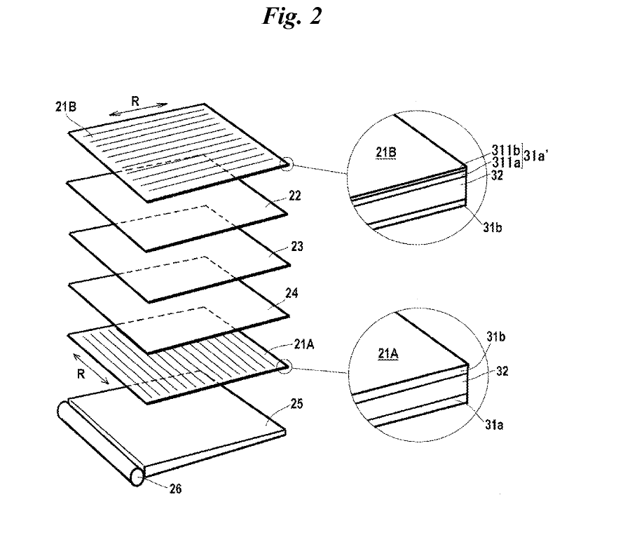

As the liquid crystal display of the present invention, an internal configuration of a typical liquid crystal display is shown in FIG. 1 and FIG. 2. In FIG. 1, a liquid crystal display is illustrated, which has polarizing plates 21A and 21B in which optical films 31a and 31b of the present invention composed of a cellulose acylate film are disposed on both surfaces of a polarizer 32. In addition, in FIG. 2, a liquid crystal display is illustrated, which is equipped with an optical film 31a′ in which a polarizing plate 21B disposed on the visual recognition side has a hard coat layer 311b on the surface on the visual recognition side of the polarizer 32 via a cellulose acylate film 311a.

Meanwhile, the configuration of each example of the liquid crystal display of the present invention is illustrated in FIG. 1 and FIG. 2, but the specific configuration of the liquid crystal display of the present invention is not particularly limited, and any configuration can be adopted. Further, the configuration shown in FIG. 2 of JP-A-2008-262161 may be preferably used.

The present invention will be described in more detail based on the following examples, but the invention is not intended to be limited thereto.

### EXAMPLES

### [Synthesis of Compound Represented by Formula (I)]

The compounds represented by formula (I) according to the present invention were synthesized in the following manner.

Synthetic examples of the representative compounds are presented below.

The exemplified compounds (A-6), (A-7), and (D-3) were synthesized, in accordance with the method described in Journal of Organic Chemistry, vol. 68, pp. 4684 to 4692 (2003).

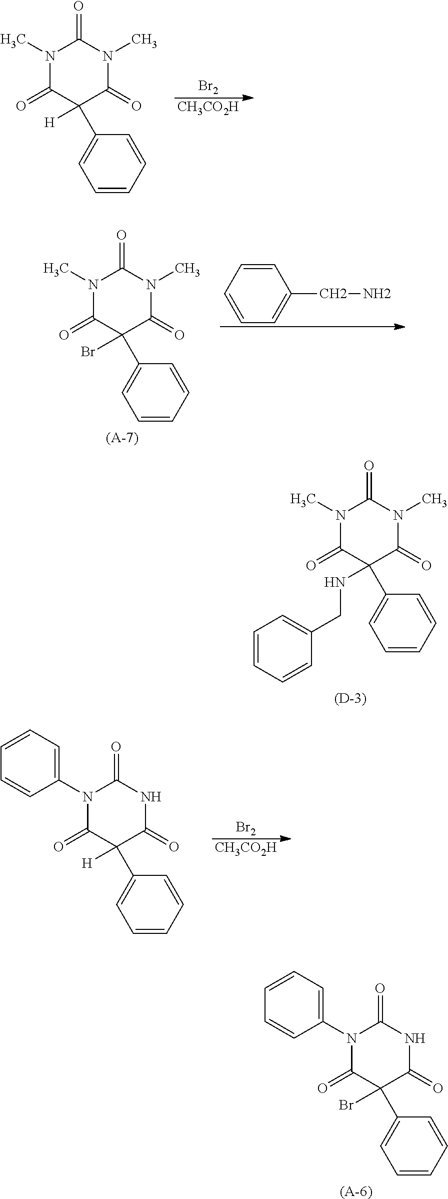

### (i) Synthesis of Exemplified Compound (A-7)

1,3-dimethyl-5-phenylbarbituric acid was subjected to bromination by bromine in acetic acid, to synthesize the Exemplified compound (A-7).

The structure of the thus-obtained compound was identified by ¹H-NMR spectrum.

### (ii) Synthesis of Exemplified Compound (D-3)

The exemplified compound (A-7) was allowed to react with benzylamine, to synthesize the exemplified compound (D-3).

The structure of the thus-obtained compound was identified by ¹H-NMR spectrum.

(iii) Synthesis of Exemplified Compound (A-6)

In the same manner as the exemplified compound (A-7), 1,5-diphenylbarbituric acid was brominated by bromine in acetic acid, to synthesize the exemplified compound (A-6).

### (iv) Synthesis of Exemplified Compound (A-4)

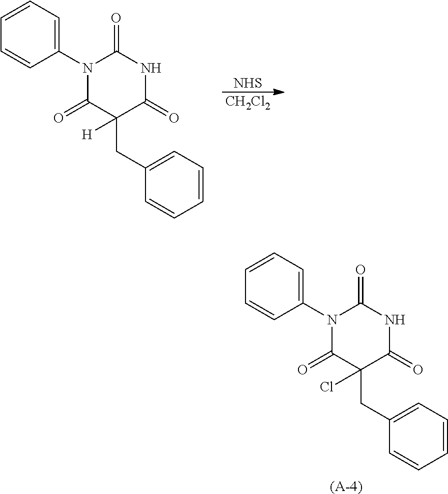

In the similar manner as the exemplified compound (A-6), the exemplified compound (A-4) was synthesized, using N-chlorosuccinic acid.

The structure of the thus-obtained compound was identified by ¹H-NMR spectrum.

### (v) Synthesis of Exemplified Compound (B-1)

By the method described in Organic Chemistry, Vol. 68, p. 4684 (2003), 1,3-dimethyl-5-benzylbarbituric acid was oxidized in acetic acid, using manganese acetate as a catalyst, to synthesize the exemplified compound (B-1).

The structure of the thus-obtained compound was identified by ¹H-NMR spectrum.

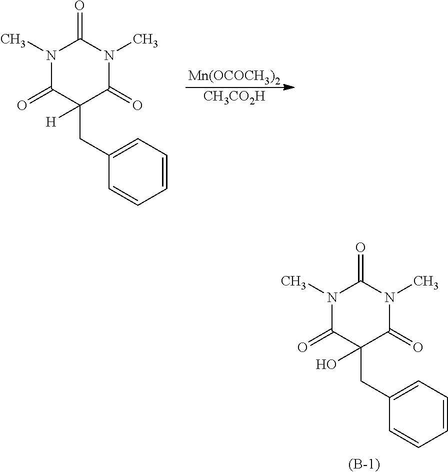

Exemplified compound (B-1)

¹H-NMR (300 MHz, DMSO-d6): δ 2.50 (s, 6H), 3.12 (s, 2H), 6.51 (s, 1H), 6.92 (m, 2H), 7.24 (m, 3H)

### (vi) Synthesis of Exemplified Compound (B-3)

The exemplified compound (B-3) was synthesized by allowing 1-benzyl-5-phenylbarbituric acid to react with meta-chloroperbenzoic acid. The structure of the thus-obtained compound was identified by ¹H-NMR spectrum.

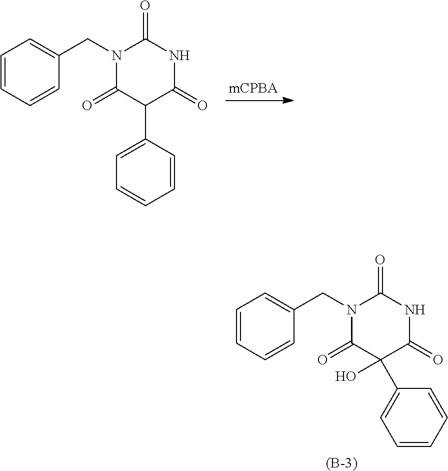 [SMILES: O=C1NC(=O)N(Cc2ccccc2)C(=O)C1c1ccccc1 ; O=C1NC(=O)C(O)(c2ccccc2)C(=O)N1Cc1ccccc1 ; "(B-3)"]

(vii) to (xv) Synthesis of Exemplified Compounds (B-4), (B-5), (B-6), (B-7), (B-13), (G-7), (G-8), (G-9), and (G-10)

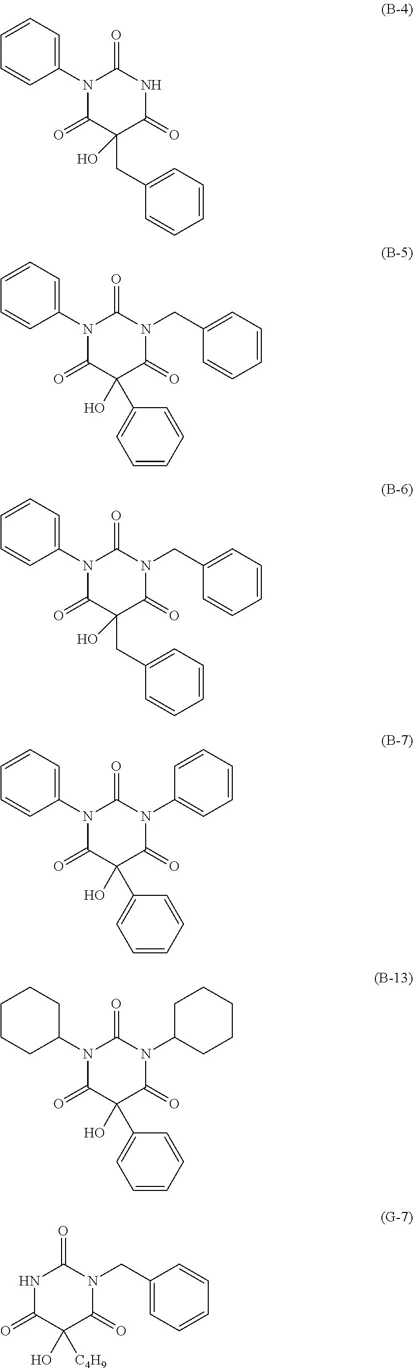

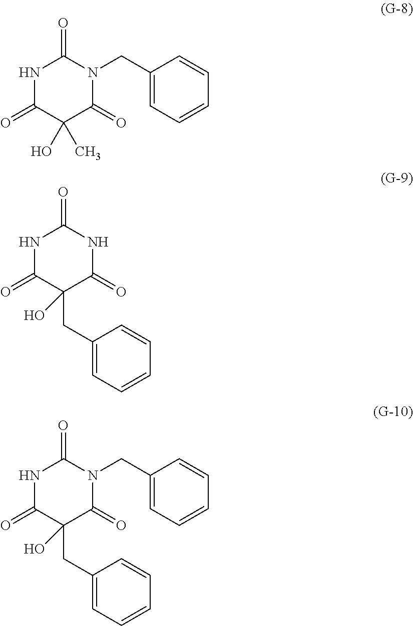

The exemplified compounds (B-4), (B-5), (B-6), (B-7), (B-13), (G-7), (G-8), and (G-10) each were synthesized in the same manner as the exemplified compound (B-3), except that 1-benzyl-5-phenylbarbituric acid the raw material was changed to 1-phenyl-5-benzylbarbituric acid, 1-benzyl-3,5-diphenylbarbituric acid, 1,5-dibenzyl-3-phenylbarbituric acid, 1,3,5-triphenylbarbituric acid, 1,3-dicyclohexyl-5-phenylbarbituric acid, 1-benzyl-5-butylbarbituric acid, 1-benzyl-5-methylbarbituric acid, and 1,5-dibenzylbarbituric acid, respectively. Further, the exemplified compound (G-9) was synthesized in the same manner as the exemplified compound (B-1), except that the raw material was changed to 5-butylbarbituric acid.

The structure of the thus-obtained compounds were identified by ¹H-NMR spectrum.

¹H-NMR data of the exemplified compounds (B-5), (B-6), (G-7), (G-8), and (G-9) are presented, as representations.

Exemplified compound (B-5)

¹H-NMR (300 MHz, CDCl₃): δ 4.13 (br, 1H), 5.12 (m, 2H), 7.08 (s, 2H), 7.26 to 7.46 (m, 13H)

Exemplified compound (B-6)

¹H-NMR (300 MHz, CDCl₃): δ 3.33 (dd, 2H), 3.65 (br, 1H), 4.94 (m, 2H), 6.88 (m, 2H), 7.14 to 7.48 (m, 13H)

Exemplified compound (G-7)

¹H-NMR (300 MHz, DMSO-d6): δ 0.75 (t, 3H), 1.04 to 1.16 (m, 4H), 1.76 (m, 2H), 4.87 (s, 2H), 6.11 (s, 1H), 7.26 to 7.33 (m, 5H), 11.59 (s, 1H)

Exemplified compound (G-8)

¹H-NMR (300 MHz, DMSO-d6): δ 1.49 (s, 3H), 4.87 (s, 2H), 6.32 (s, 1H), 7.29 (m, 5H), 11.50 (s, 1H)

Exemplified compound (G-9)

¹H-NMR (300 MHz, DMSO-d6): δ 3.12 (s, 2H), 6.40 (s, 1H), 7.03 (m, 2H), 7.28 (m, 3H), 11.21 (s, 2H)

(xvi) Synthesis of Exemplified Compound (B-9)

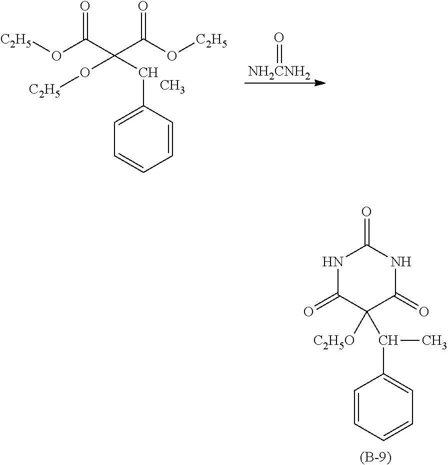

By the method described in Journal of Medicinal Chemistry, Volume 17, p. 1194 (1974), the exemplified compound (B-9) was synthesized by the cyclization reaction of diethyl ethoxy- 1 -phenylethylmalonate and urea.

The structure of the thus-obtained compound was identified by ¹H-NMR spectrum.

(xvii) Synthesis of Exemplified Compound (B-11)

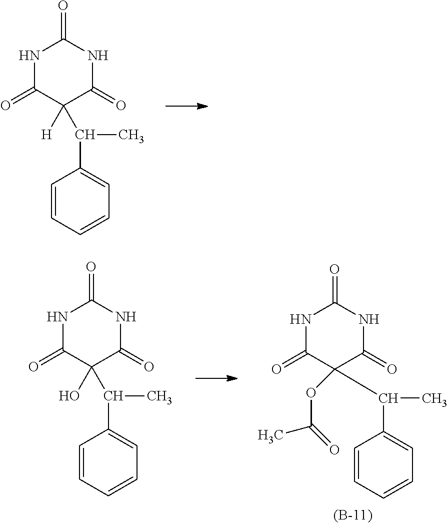

By the method described in Journal of Medicinal Chemistry, Volume 17, p. 1194 (1974), 5-(1-phenylethyl)barbituric acid was acetylated via the 5-position hydroxy form, to synthesize the exemplified compound (B-11).

The structure of the thus-obtained compound was identified by ¹H-NMR spectrum.

### EXAMPLE 1

### (A) Preparation and Evaluation of Cellulose Acylate Film (Optical Film)—1—

### (Preparation of Cellulose Acetate)

Cellulose acylate having 2.87 of the total acetyl substitution degree (B) was prepared in the following manner.

Sulfuric acid (7.8 parts by mass with respect to 100 parts by mass of cellulose) was added as a catalyst, and an acetic acid was added, and the cellulose was acetylated by the reaction at 40° C. Further, after acetylation, ripening (aging) was conducted at 40° C. Further, a low molecular component part of the cellulose acetate was washed with acetone and removed.

### (Optical Film: Preparation of Single-Layer Cellulose Acylate Film)

The following composition was poured into a mixing tank, and each of components was dissolved by stirring, to prepare a cellulose acetate solution.

Composition of cellulose acetate solution

| | | |
|---|---|---|
| Cellulose acetate with the total acetyl substitu- | 100.0 | parts by mass |
| tion degree (B) 2.87 and the polymerization | | |
| degree 370 | | |
| Compound represented by formula (I) shown in | 10.0 | parts by mass |
| Table 2 | | |
| Methylene chloride (the first solvent) | 402.0 | parts by mass |
| Methanol (the second solvent) | 60.0 | parts by mass |

After casting the cellulose acetate solution using a band casting machine, followed by drying at 100° C. so as to have a residual solvent content of 40%, the film was peeled off The film thus peeled-off was further dried for 20 minutes at an ambient temperature of 140° C. In this manner, the optical film Nos. 101 to 103 of the present invention and the optical film Nos. c01 to c03 for comparison were produced, respectively. The film thickness of each of the thus-obtained optical films (cellulose acylate films) was 60 μm.

Meanwhile, the optical film is also referred to as the polarizing plate protective film, hereinafter.

### (Measurement of Film Hardness)

The film hardness (surface hardness) of the thus-produced optical film Nos. 101 to 103 of the present invention and the optical film Nos. c01 to c03 for comparison was measured.

A surface of a sample fixed on a glass substrate was measured, using a FISCHERSCOPE H100Vp-type hardness tester, manufactured by Fischer Instruments, under conditions of loading time period 10 seconds, creep time period 5 seconds, unloading time period 10 seconds, and maximum load 50 mN, by a Knoop indenter, in which the minor axis direction of the Knoop indenter was disposed in parallel to the transporting direction (MD direction: the test direction in the pencil hardness test) in the film formation of the cellulose acylate film. The hardness was calculated from the relationship of the maximum load with the contact area between the indenter and the sample obtained from the indentation depth, and the average value of those five points were defined as the surface hardness, respectively.

Also, a surface of the sample fixed on a glass substrate was measured, in accordance with the method of JIS Z 2251, using a FISCHERSCOPE H100Vp-type hardness tester, manufactured by Fischer Instruments, under the conditions of loading time period 10 seconds, creep time period 5 seconds, unloading time period 10 seconds, and indentation load 50 mN, and the Knoop hardness was calculated from the relationship of the maximum load with the contact area between the indenter and the sample obtained from the indentation depth, respectively. JIS Z 2251 is the Japanese Industrial Standard, which is defined based on ISO 4545-1 and ISO 4545-4.

Further, measurements for Knoop hardness were carried out in the total 18 directions, when rotating the Knoop indenter by 10° from 0° to 180°, the each measurement was carried out at the same press position, to obtain the minimum value of Knoop hardness. The minimum value corresponded to the surface hardness (the Knoop hardness) obtained where the minor axis direction of the Knoop indentor was disposed in parallel to the conveying direction (MD direction: the test direction in the pencil hardness test) when forming the cellulose acylate film. The unit was expressed in N/mm²

The value was calculated as the hardness improving effect by dividing the value of Knoop hardness in the case of adding an additive by the value of Knoop hardness in the case of not adding the additive, and the evaluation was performed according to the following criteria.

Meanwhile, the hardness as a cellulose acetate film is high when the evaluation is “C” or higher (i.e. A, B or C), and the cellulose acetate film is sufficiently practical from the viewpoint of workability. Evaluation criteria of the surface hardness
A: The value was 1.15 times or more than the value of Knoop hardness in the case of not adding the additive.
B: The value was 1.10 times or more but less than 1.15 times than the value of

Knoop hardness in the case of not adding the additive.
C: The value was 1.03 times or more but less than 1.10 times than the value of Knoop hardness in the case of not adding the additive.
D: The value was less than 1.03 times than the value of Knoop hardness in the case of not adding the additive.

### (Evaluation of Coloration of Film)

To each optical film of the present invention produced above, light irradiation was performed for 120 hours, under the conditions of irradiance 150 W/m², black panel temperature 63° C., and relative humidity 50%, using a super xenon weathermeter (SX75, manufactured by Suga Test Instruments Co., Ltd.). After that, the hue b* was measured, using a spectrophotometer UV3150, manufactured by Shimadzu Corporation. As the hue b*value increases in the negative direction, a blue color of the transmitted light increases, while on the other hand, as the hue b*value increases in the positive direction, a yellow color increases. The change in the value of b* before and after the light irradiation was denoted as Ab*, and this was used as an index for coloration by light. The results of this measurement were evaluated according to the following criterion.
A: Δb* was less than 0.04.
B: Δb* was 0.04 or more, and less than 0.06.
C: Δb* was 0.06 or more, and less than 0.08.
D: Δb* was 0.08 or more.

These results are shown together in Table 2.

TABLE 2

| | Additive | | | Coloration of | |
|---|---|---|---|---|---|
| | | Addition | | film (with | |
| | | amount ¹⁾ | Film | the lapse | |
| Film | Compound | (mass | hard- | of time | |
| No. | No. | parts) | ness | period, aging) | Remarks |
| 101 | A-4 | 10 | B | A | This invention |
| 102 | B-3 | 10 | A | A | This invention |
| 103 | B-4 | 10 | A | A | This invention |
| c01 | Compound | 10 | A | C | Comparative |
| | (R-1) for | | | | example |
| | comparison | | | | |
| c02 | Compound | 10 | C | A | Comparative |
| | (R-2) for | | | | example |
| | comparison | | | | |
| c03 | None | 0 | — | A | Reference |
| | | | | | example |

Note:
¹⁾ Addition amount with respect to 100 parts by mass of the cellulose acylate

Herein, the compounds (R-1) and (R-2) for comparison are the compounds (2) and (19) described in JP-A-2011-126968, respectively.

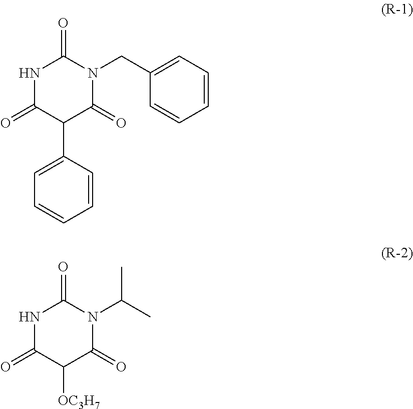

As can be seen from Table 2, it has been found that all of the optical film Nos. 101 to 103 of the present invention are excellent in the effects of suppressing the coloration of the film by light, and further exhibit a favorable hardness.

All of the optical film Nos. 101 to 103 of the present invention exhibit a higher surface hardness, as compared to Compound (R-2) for comparison (the optical film No. c02 for comparison). It is considered that this is because the compound represented by formula (I) according to the present invention has a ring structure or a polar group and thus the interaction thereof with the cellulose acetate is further strengthened.

In addition, it shows that the coloration of the film by light is suppressed in all of the optical film Nos. 101 to 103 of the present invention, as compared to Compound (R-1) for comparison (the optical film No. c01 for comparison).

### EXAMPLE 2

### (B) Preparation and Evaluation of Cellulose Acylate Film (Optical Film)—2—

The optical film Nos. 111 to 120 of the present invention were produced in the same manner as in Example 1, except that the kind of the compound represented by formula (I) was changed to those presented in Table 3. The evaluation on each property was performed in the same manner as in Example 1.

TABLE 3

| | Additive | | | | |
|---|---|---|---|---|---|
| | | Addition | Film | | |
| Film | Compound | amount ¹⁾ | hard- | Coloration of | |
| No. | No. | (mass parts) | ness | film (aging) | Remarks |
| 111 | A-6 | 10 | B | A | This invention |
| 112 | A-7 | 10 | B | A | This invention |
| 113 | B-1 | 10 | B | A | This invention |
| 114 | B-5 | 10 | A | A | This invention |
| 115 | B-6 | 10 | A | A | This invention |
| 116 | B-7 | 10 | A | A | This invention |
| 117 | B-9 | 10 | A | A | This invention |
| 118 | B-11 | 10 | A | A | This invention |
| 119 | B-13 | 10 | A | A | This invention |
| 120 | D-3 | 10 | A | B | This invention |

Note:
¹⁾ Addition amount with respect to 100 parts by mass of the cellulose acylate

As can be seen from Table 3, in all of the optical film Nos. 111 to 120 of the present invention, the surface hardness was high and the film coloration by light was suppressed. Thus, it has been found that all of the compounds represented by formula (I) contribute to the hardness exhibiting property and suppression of the film coloration.

### EXAMPLE 3

### (C) Preparation and Evaluation of Cellulose Acylate Film (Optical Film)—3—

The optical film Nos. 131 to 137 of the present invention were produced in the same manner as in Example 1, except that the degree of substitution of cellulose acylate, the kind of the compound represented by formula (I), and the film thickness of the cellulose acylate film, each were changed to those shown in Table 4.

The evaluation on each property was performed in the same manner as in Example 1.

TABLE 4

| | Cellulose acylate | Additive | | Film | | Coloration | |
|---|---|---|---|---|---|---|---|
| Film | Acetyl substitution | Compound | Addition | thickness | Film | of film | |
| No. | degree (B) | No. | amount¹⁾ | μm | hardness | (aging) | Remarks |
| 131 | 2.42 | B-1 | 10 | 63 | B | A | This invention |
| 132 | 2.42 | B-3 | 10 | 55 | A | A | This invention |
| 133 | 2.42 | B-6 | 10 | 57 | A | A | This invention |
| 134 | 2.77 | B-6 | 10 | 62 | A | A | This invention |
| 135 | 2.93 | B-1 | 10 | 61 | B | A | This invention |
| 136 | 2.93 | B-3 | 10 | 63 | A | A | This invention |
| 137 | 2.93 | B-6 | 10 | 61 | A | A | This invention |

Note:
¹⁾Addition amount with respect to 100 parts by mass of the cellulose acylate

As can be seen from Table 4, it has been found that the compounds according to the present invention are effective in the hardness and the suppressing of the film coloration, regardless of the degree of substitution of the cellulose acylate

### Example 4

### (D) Preparation and Evaluation of Cellulose Acylate Film (Optical Film)—4—

The optical film Nos. 141 to 145 of the present invention and the optical film Nos. c41 to c43 for comparison were, respectively, produced in the same manner as in Example 1, except that the kind of the cellulose acylate, the kind of the respective additive, and the film thickness of the cellulose acylate film, were changed to those shown in Table 5.

The evaluation on each property was performed in the same manner as in Example 1. However, upon the evaluation on the surface hardness, the indentation load was changed depending on the film thickness as described below.

### (Evaluation on Surface Hardness)

The film hardness (surface hardness) was measured in the same manner as in the method described in Example 1, except that the indentation load of the cellulose acylate film obtained above was set to 20 mN.

The value of Knoop hardness of each of the films was compared to the value of Knoop hardness of the films produced without adding any additive, and the evaluation was performed according to the following criteria.
A: The value was 1.15 times or more than the value of Knoop hardness in the case of not adding the additive.
B: The value was 1.10 times or more but less than 1.15 times than the value of Knoop hardness in the case of not adding the additive.
C: The value was 1.03 times or more but less than 1.10 times than the value of Knoop hardness in the case of not adding the additive.
D: The value was less than 1.03 times than the value of Knoop hardness in the case of not adding the additive.

TABLE 5

| | Cellulose acylate | Additive | | Film | | Coloration | |
|---|---|---|---|---|---|---|---|
| Film | Acetyl substitution | Compound | Addition | thickness | Film | of film | |
| No. | degree (B) | No. | amount¹⁾ | μm | hardness | (aging) | Remarks |
| 141 | 2.86 | B-1 | 10 | 41 | B | A | This invention |
| 142 | 2.86 | B-4 | 10 | 30 | A | A | This invention |
| 143 | 2.86 | B-5 | 10 | 24 | A | A | This invention |
| 144 | 2.86 | B-6 | 10 | 26 | A | A | This invention |
| 145 | 2.86 | B-7 | 10 | 25 | A | A | This invention |
| c41 | 2.86 | Compound (R-1) | 10 | 28 | A | C | Comparative |
| | | for comparison | | | | | example |
| c42 | 2.86 | Compound (R-2) | 10 | 26 | C | A | Comparative |
| | | | | | | | example |
| c43 | 2.86 | None for | 10 | 24 | — | A | Reference |
| | | comparison | | | | | example |

Note:
¹⁾Addition amount with respect to 100 parts by mass of the cellulose acylate

As can be seen from Table 5, it has been found that in the film containing the compound according to the present invention, a preferred surface hardness improving effect and a preferred effect of suppressing of the coloration by light can be exhibited, even when the film is thinned.

### Example 5

### (Preparation of Optical Film with Hard Coat Layer)

The hard coat layer solution of the composition described below was applied on the surface of each of the single-layer optical films produced in Examples 1 and 2, followed by irradiation with an ultraviolet ray to cure, thereby to produce optical films with hard coat layers, with the hard coat layers each being formed at thickness 6 μm.

Composition of hard coat layer solution

| | | |
|---|---|---|
| Monomer, pentaerythritol | 53.5 | parts by mass |
| triacrylate/pentaerythritol | | |
| tetraacrylate (mixing mass ratio 3/2)) | | |
| UV initiator, Irgacure ™907 (manufactured | 1.5 | parts by mass |
| by BASF) | | |
| Ethyl acetate | 45 | parts by mass |

### <Evaluation of Pencil Hardness>

Each of the optical films provided with the hard coat layers was conditioned at 25° C. and relative humidity 60% for 2 hours, and then tested according to the pencil hardness test method stipulated in JIS-K5400, using a test pencil defined in JIS-S6006. Using a weight of 500 g, the surface of the hard coat layer was scratched repeatedly for a total of five times with a pencil having a different hardness, and the hardness of the tested pencil with which the sample had been given one scratch was determined. The scratch defined in JIS-K5400 is a breakage of the coating film or the scratch of the coating film and does not include any dent of the coating film. However, in this test, the judge was made that the scratch included a dent of the coating layer. As a result, it has been found that all of the optical films with hard coat layers based on the optical film Nos. 101 to 103 and 111 to 120 of the present invention exhibit a favorable value of 3H.

### Example 6

### <Performance Evaluation as Polarizing Plate>

### (Saponification Treatment of Polarizing Plate Protective Film)

The polarizing plate protective film, which is composed of the optical film No. 101 of the present invention produced in Example 1, was soaked in a 2.3 mol/L sodium hydroxide aqueous solution, at 55° C. for 3 minutes. The film was then washed in a water-washing bath at room temperature and neutralized with 0.05 mol/L sulfuric acid at 30° C. The film was, again, washed in a water-washing bath at room temperature and further dried by warm air at 100° C. In this manner, the surface of the polarizing plate protective film composed of each of the optical films produced in Examples 1 to 4 was subjected to the saponification treatment. Meanwhile, the polarizer to be used was a commonly used polarizer as described in the section of <<Polarizing plate>> above. (Preparation of polarizing plate)

A polarizer was prepared by adsorbing iodine onto a stretched polyvinyl alcohol film.

The polarizing plate protective film No. 101, which was produced in Example 1 and had been subjected to the saponification treatment, was stuck to one side of the polarizer, with a polyvinyl alcohol-based adhesive. A commercially-available cellulose triacetate film (FUJITAC TD8OUF, manufactured by Fujifilm Corporation) was also subjected to the saponification treatment. With a polyvinyl alcohol-based adhesive, the thus-obtained commercially-available cellulose triacetate film subjecting to the saponification treatment was stuck to the other side of the polarizer, which was opposite to the side to which the polarizing plate protective film No. 101 after the saponification treatment was stuck.

At that time, the transmission axis of the polarizer and the slow axis of the polarizing plate protective film No. 101, which was produced in Example 1 and had been subjected to the saponification treatment, were disposed so that they were parallel to one another. Further, the transmission axis of the polarizer and the slow axis of the commercially-available cellulose triacetate film having been subjected to the saponification treatment were also disposed so that they were perpendicular to one another.

Thus, the polarizing plate 101 of the present invention was produced.

Also with respect to each of the polarizing plate protective film Nos. 102 to 103, 111 to 120, 131 to 137, and 141 to 145 and the polarizing plate protective film Nos. c01 to c03, and c41 to c43 for comparison, the saponification treatment and the preparation of polarizing plate were conducted in the same manner as above, to produce each of polarizing plate Nos. 102 to 103, 111 to 120, 131 to 137, and 141 to 145 of the present invention and polarizing plate Nos. c01 to c03, and c41 to c43 for comparison, respectively.

The polarizing plate of the present invention reflected the performance of the contained optical film of the present invention, and exhibited excellent performance.

As a result, it is possible to produce a liquid crystal display exhibiting excellent performance as described above, by using the optical film of the present invention, and the polarizing plate using the same.

### Example 7

(Combination with Plasticizer)

In the following manner, a polarizing plate was produced, and the polarizing plate durability was evaluated.

### (Preparation of Cellulose Acylate Solution 71)

The following composition was poured into a mixing tank, and each of components was dissolved by stirring, to prepare a cellulose acylate solution 71.

Composition of cellulose acylate solution 71

| | | |
|---|---|---|
| Cellulose acetate with the acetyl substitution | 100.0 | parts by mass |
| degree 2.87 and the polymerization degree 370 | | |
| Plasticizer: Hydrophobizing agent (1) | 10.0 | parts by mass |
| Polycondensate of phthalic acid and ethanediol, | | |
| with the terminal an acetic ester group and | | |
| the number-average molecular weight 800 | | |
| Methyl chloride (the first solvent) | 398.8 | parts by mass |
| Methanol (the second solvent) | 58.2 | parts by mass |

### (Preparation of Matting Agent Solution 72)

The following composition was poured into a dispersing machine, and each of components was dissolved by stirring, to prepare a matting agent solution 72.

Composition of matting agent solution 72

| | | |
|---|---|---|
| Silica particles with the average particle size | 2.0 | parts by mass |
| 20 nm (AEROSIL R972, manufactured by | | |
| Nippon Aerosil) | | |
| Methylene chloride (the first solvent) | 75.5 | parts by mass |
| Methanol (the second solvent) | 11.3 | parts by mass |
| Cellulose acylate solution 71 | 0.9 | part by mass |

### (Preparation of Polarizer Durability Enhancer Solution 73)

The following composition was poured into a mixing tank, and each of components was dissolved by stirring while heating, to prepare a polarizer durability enhancer solution 73.

Composition of polarizer durability enhancer solution 73

| | | |
|---|---|---|
| Exemplified compound (B-3) | 20.0 | parts by mass |
| Methylene chloride (the first solvent) | 73.5 | parts by mass |
| Methanol (the second solvent) | 6.4 | parts by mass |

(Preparation of Ultraviolet Absorber Solution 74)

The following composition was poured into a mixing tank, and each of components was dissolved by stirring while heating, to prepare an ultraviolet absorber solution 74.

Composition of ultraviolet absorber solution 74

| | |
|---|---|
| Ultraviolet absorber (UV-1) | 10.0 parts by mass |
| Methylene chloride (first solvent) | 78.3 parts by mass |
| Methanol (second solvent) | 11.7 parts by mass |

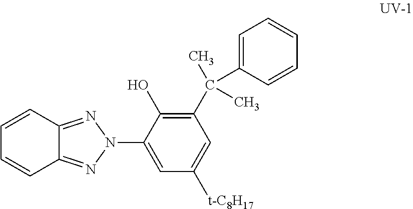

### <Casting>

Each of 1.3 parts by mass of the matting agent solution 72, 3.3 parts by mass of the polarizer durability enhancer solution 73, and 4.0 parts by mass of the ultraviolet absorber solution 74 were filtered, followed by mixing by using an in-line mixer, and 91.4 parts by mass of the cellulose acylate solution 71 was added thereto, followed by mixing the resultant mixture by using an in-line mixer. A band casting device was utilized, to cast the thus-prepared dope on a casting support made of stainless steel (support temperature 22° C.). The resultant film was peeled-off in a state where the amount of the remaining solvent in the dope was approximately 20% by mass, followed by drying while being stretched by 1.10 times (10%) in the width direction at a temperature of 120° C., in a state where the amount of the remaining solvent was 5% by mass to 10% by mass, while gripping both ends of the film in the width direction with a tenter. After that, the film was further dried, by letting it transport between rolls of the heat treatment apparatus, to prepare the optical film No. 701. The thickness of the thus-obtained optical film was 23 μm, and the width thereof was 1,480 mm.

Moreover, the optical film Nos. 702 to 705, and 711 to 715 of the present invention and the optical film Nos. c71 and c72 for comparison were produced in the same manner as the optical film of No. 701, except that the kind and the addition amount of the exemplified compound to be added, and the kind and the addition amount of the plasticizer in the optical film of No. 701 were changed to those shown in Table 6.

In addition, the optical film of No. c73 for comparison was produced in the same manner as the optical film of No. 701, except that the polarizer durability enhancer solution 73 was not mixed in the optical film of No. 701.

### (Description of Additives)

Hydrophobizing agent 1: a polycondensate of phthalic acid and ethanediol (the end thereof is a acetic ester group, and the number-average molecular weight is 800)
Polycondensation polymer (A): a polyester obtained from adipic acid and ethanediol (the end thereof is a hydroxy group) (the number-average molecular weight of 1,000)
MONOPET (registered trademark) SB (a plasticizer): manufactured by Dai-Ichi Kogyo Seiyaku Co., Ltd.
SAIB-100 (a plasticizer): manufactured by Eastman Chemical Company

In the same manner as the optical film of No. 701, the optical film of No. 801 of the present invention was produced, by casting and drying such that the film thickness of the optical film to be obtained would be 40 μm and the width thereof would be 1,480 mm. The optical film Nos. 802 to 805 of the present invention and the optical film Nos. c81 to c83 for comparison were, respectively, produced in the same manner as the optical film of No. 801, except that the kind and the addition amount of the additive were changed to those presented in Table 6 instead of the exemplified compound (B-3) in the optical film of No. 801. Further, in the same manner as the optical film of No. 711, the optical film of No. 811 of the present invention was produced, by casting and drying such that the film thickness of the optical film to be obtained would be 40 μm and the width thereof would be 1,480 mm. The optical film Nos. 812 to 815 of the present invention were produced in the same manner as the optical film of No. 811, except that the kind and the addition amount of the additive were changed to those presented in Table 6.

[Evaluation on Film Hardness and Durability (Coloration with the Lapse of Time Period) to Film Coloration by Light]

Each of the optical films produced in this manner was evaluated by the same evaluation method and evaluation criteria as in Example 4.

### <Preparation of Polarizing Plate>

The polarizing plate protective film composed of the optical film Nos. 701 to 705, 711 to 715, 801 to 805, and 811 to 815 of the prevent invention and the optical film Nos. c71 to c73, and c81 to c83 for comparison produced above, respectively, was soaked in a 2.3 mol/L sodium hydroxide aqueous solution, at 55° C. for 3 minutes. The film was then washed in a water-washing bath at room temperature, followed by neutralization with 0.05 mol/L sulfuric acid at 30° C. The film was, again, washed in a water-washing bath at room temperature, followed by further drying by warm air at 100° C. Thus, each saponified polarizing plate protective film was obtained.

### (Preparation of Polarizing Plate)

The polarizing plate, corresponding to the optical film Nos. 701 to 705, 711 to 715, 801 to 805, and 811 to 815 of the present invention and the optical film Nos. c71 to c73, and c81 to c83 for comparison, were produced, respectively, in the same manner as in Example 2, except for using the respective thus-saponified polarizing plate protective film having been subjected to the saponification treatment.

[Evaluation of Polarizing Plate Durability]

The polarizing plate durability test was conducted in below, in the form in which the polarizing plate and a glass were stuck together via an adhesive.

Two samples (about 5 cm x 5 cm) were prepared, in which the polarizing plate was stuck on the glass such that the optical film of the present invention would be on the air interface side. The single-plate perpendicular transmittance was measured, by setting the optical film side of the film of this sample to direct toward a light source. The two samples were measured, and the average of the thus-measured values was used as a perpendicular transmittance of the polarizing plate. The perpendicular transmittance of the polarizing plate was measured in the range of from 380 nm to 780 nm, using the automatic polarizing film measuring device VAP-7070, manufactured by JASCO Corporation, and the value measured at 410 nm was adopted. Thereafter, the perpendicular transmittance was measured in the same manner, after storing with the lapse of time period (aging), under the following conditions. A change of the perpendicular transmittance before and after aging was measured. By taking the change as the polarizing plate durability, evaluation was conducted in accordance with the following criteria.

It is noted that the relative humidity, under the environment without humidity conditioning, was in the range of from 0% RH to 20% RH.

The lapse of time period conditions and the evaluation conditions of the polarizing plates corresponding to the optical film Nos. 701 to 705, 711 to 715, and c71 to c73

### —The Lapse of Time Period Conditions—

These samples were stored for 500 hours under the environment of 60° C. and relative humidity 95%RH.
A+: The change of the perpendicular transmittance before and after the lapse of time period (aging) was less than 0.8%.
A: The change of the perpendicular transmittance before and after aging was 0.8% or more and less than 1.0%.
B: The change of the perpendicular transmittance before and after aging was 1.0% or more and less than 1.2%.
C: The change of the perpendicular transmittance before and after aging was 1.2% or more.

The lapse of time period conditions and the evaluation conditions of the polarizing plates corresponding to the optical film Nos. 801 to 805, 811 to 815, and c.81 to c.83 —The Lapse of Time Period Conditions—

These samples were stored for 500 hours, under the environment of 60° C. and relative humidity 95%RH.
A+: The change of the perpendicular transmittance before and after aging was less than 0.6%.
A: The change of the perpendicular transmittance before and after aging was 0.6% or more and less than 0.7%.
B: The change of the perpendicular transmittance before and after aging was 0.7% or more and less than 0.8%.
C: The change of the perpendicular transmittance before and after aging was 0.8% or more.

The obtained results are shown together in Table 6.

TABLE 6

| | Additive | | Plasticizer | | | Coloration | Polarizing | |
|---|---|---|---|---|---|---|---|---|
| Film | Compound | Addition | Compound | Addition | Film | of film | plate | |
| No. | No. | amount¹⁾ | No. | amount¹⁾ | hardness | (aging) | durability | Remarks |
| 701 | B-3 | 4 | Hydrophobizing agent (1) | 10 | A | A | A+ | This invention |
| 702 | B-4 | 4 | Hydrophobizing agent (1) | 10 | A | A | A+ | This invention |
| 703 | B-5 | 4 | Hydrophobizing agent (1) | 10 | A | A | A+ | This invention |
| 704 | B-6 | 4 | Hydrophobizing agent (1) | 10 | A | A | A+ | This invention |
| 705 | A-4 | 4 | Hydrophobizing agent (1) | 10 | A | A | A | This invention |
| 711 | B-3 | 4 | MONOPET SB/SAIB-100 | 9/3 | A | A | A | This invention |
| 712 | B-4 | 2 | MONOPET SB/SAIB-100 | 9/3 | A | A | A | This invention |
| 713 | B-5 | 2 | MONOPET SB/SAIB-100 | 9/3 | A | A | B | This invention |
| 714 | B-6 | 1 | MONOPET SB/SAIB-100 | 9/3 | A | A | B | This invention |
| 715 | A-4 | 1 | MONOPET SB/SAIB-100 | 9/3 | A | A | B | This invention |
| c71 | Compound (R-1) | 4 | Hydrophobizing agent (1) | 10 | A | C | A | Comparative |
| | for comparison | | | | | | | example |
| c72 | Compound (R-2) | 4 | Hydrophobizing agent (1) | 10 | C | A | C | Comparative |
| | for comparison | | | | | | | example |
| c73 | None | 0 | Hydrophobizing agent (1) | 10 | — | A | C | Comparative |
| | | | | | | | | example |
| 801 | B-3 | 4 | Hydrophobizing agent (1) | 10 | A | A | A+ | This invention |
| 802 | B-4 | 4 | Hydrophobizing agent (1) | 10 | A | A | A+ | This invention |
| 803 | B-5 | 4 | Hydrophobizing agent (1) | 10 | A | A | A+ | This invention |
| 804 | B-6 | 4 | Hydrophobizing agent (1) | 10 | A | A | A+ | This invention |
| 805 | A-4 | 4 | Hydrophobizing agent (1) | 10 | A | A | A+ | This invention |
| 811 | B-5 | 4 | MONOPET SB/SAIB-100 | 9/3 | A | A | A | This invention |
| 812 | B-6 | 4 | MONOPET SB/SAIB-100 | 9/3 | A | A | A | This invention |
| 813 | B-5 | 4 | Polycondensation polymer (A) | 12 | A | A | A | This invention |
| 814 | B-6 | 4 | Polycondensation polymer (A) | 12 | A | A | A | This invention |
| 815 | A-4 | 4 | Polycondensation polymer (A) | 12 | A | A | A | This invention |
| c81 | Comparative | 4 | Hydrophobizing agent (1) | 9/3 | A | C | A | Comparative |
| | compound (R-1) | | | | | | | example |
| c82 | Comparative | 4 | Hydrophobizing agent (1) | 9/3 | C | A | C | Comparative |
| | compound (R-2) | | | | | | | example |
| c83 | None | 0 | Hydrophobizing agent (1) | 9/3 | — | A | C | Comparative |
| | | | | | | | | example |

Note:
¹⁾Addition amount with respect to 100 parts by mass of the cellulose acylate

As can be seen from Table 6, it has been found that all of the polarizing plate protective films, which are the optical films of the present invention containing the compounds represented by formula (I) according to the present invention, are excellent in the polarizing plate durability with the lapse of time period, as compared to the optical film Nos. c73 and c83 for comparison, which were produced without adding any additive, and thus it is possible to effectively suppress the degradation of polarizer. In addition to this, all of the polarizing plate protective films, which are the optical films of the present invention, are less coloration by light with the lapse of time period.

In contrast to this, the effects of improving the polarizing plate durability or the suppression of coloration with the lapse of time period by light are not obtained, in the polarizing plate protective films, which were the optical films containing Compound (R-1) or (R-2) for comparison.

Both of the polarizing plate protective film Nos. c73 and c83, which were the optical films for comparison containing neither compound represented by formula (I) according to the present invention nor compound for comparison, were poor in the polarizing plate durability, as compared to the polarizing plate protective films, which were the optical films of the present invention.

As a result, it is possible to produce a liquid crystal display exhibiting excellent performance as described above, by using the polarizing plate of the present invention.

### EXAMPLE 8

(Combination use with Compound Represented by Formula (A))

The optical film Nos. 901 to 917 of the present invention were produced in the same manner as in Example 7, except that the kind and addition amount of the compound represented by formula (I) were changed to those presented in Table 7, and further that the compound represented by formula (A) or another additive was added as presented in Table 7. Then, the polarizing plate durability was evaluated under the following conditions.

### —The Lapse of Time Period Conditions—

These samples were stored for 500 hours under the environment of 60° C. and relative humidity 95% RH.
A++: The change of the perpendicular transmittance before and after aging was less than 0.7%.
A+: The change of the perpendicular transmittance before and after aging was 0.7% or more and less than 0.8%.
A: The change of the perpendicular transmittance before and after aging was 0.8% or more and less than 1.0%.
B: The change of the perpendicular transmittance before and after aging was 1.0% or more and less than 1.2%.
C: The change of the perpendicular transmittance before and after aging was 1.2% or more.

The thus-obtained results are shown together in Table 7.

TABLE 7

| | Additive | | Other additive | | Plasticizer | | Polarizing | |
|---|---|---|---|---|---|---|---|---|
| Film | Compound | Addition | Compound | Addition | Compound | Addition | plate | |
| No. | No. | amount 1) | No. | amount¹⁾ | No. | amount¹⁾ | durability | Remarks |
| 901 | B-3 | 2 | BA-10 | 2 | Hydrophobizing agent (1) | 10 | A+ | This invention |
| 902 | A-4 | 2 | BA-11 | 4 | Hydrophobizing agent (1) | 10 | A+ | This invention |
| 903 | B-5 | 2 | BA-22 | 2 | Hydrophobizing agent (1) | 10 | A+ | This invention |
| 904 | B-6 | 1 | BA-23 | 4 | Hydrophobizing agent (1) | 10 | A++ | This invention |
| 905 | B-6 | 0.5 | BA-23 | 3.5 | Hydrophobizing agent (1) | 10 | A++ | This invention |
| 906 | B-6 | 0.5 | BA-23 | 3.5 | MONOPET SB/SAIB-100 | 9/3 | A++ | This invention |
| 907 | B-5 | 0.5 | BA-22 | 3.5 | Hydrophobizing agent (1) | 10 | A++ | This invention |
| | | | IRGANOX1010 | 0 .1 | | | | |
| 908 | B-6 | 0.25 | BA-23 | 4 | Hydrophobizing agent (1) | 10 | A++ | This invention |
| | | | Reductone L | 0.2 | | | | |
| 909 | B-3 | 0.5 | BA-10 | 3.5 | MONOPET SB/SAIB-100 | 9/3 | A++ | This invention |
| | | | TINUVIN123 | 0.2 | | | | |
| 910 | B-6 | 0.5 | BA-23 | 3.5 | Hydrophobizing agent (1) | 10 | A++ | This invention |
| | | | TINUVIN123 | 0.18 | | | | |
| | | | Tekuran DO | 0.02 | | | | |
| 911 | B-6 | 0.2 | BA-23 | 3.8 | MONOPET SB/SAIB-100 | 9/3 | A++ | This invention |
| | | | TINUVIN123 | 0.18 | | | | |
| | | | Tekuran DO | 0.02 | | | | |
| 912 | G-7 | 0.5 | BA-36 | 3.5 | MONOPET SB/SAIB-100 | 9/3 | A++ | This invention |
| | | | Poe Bm K-37V | 0.1 | | | | |
| 913 | B-6 | 0.5 | BA-23 | 3.5 | | | | |
| | | | TINUVIN123 | 0. | Hydrophobizingagent (1) | 10 | A++ | This invention |
| | | | CHELEST PH-540 | 0.003 | | | | |
| | | | EPOMIN PP-061 | 0.024 | | | | |
| 914 | B-6 | 0.5 | BA-23 | 5.5 | MONOPETSB/SAIB-100 | 6/2 | A++ | This invention |
| | | | TINUVIN123 | 0. | | | | |
| | | | CHELEST PH-540 | 0.005 | | | | |
| | | | EPOMIN PP-061 | 0.036 | | | | |
| 915 | G-8 | 0.5 | BA-37 | 3.5 | Hydrophobizing agent (1) | 10 | A++ | This invention |
| | | | Poe Bm K-37V | 0.2 | | | | |
| 916 | G-9 | 0.1 | BA-2 | 1.9 | Hydrophobizing agent (1) | 10 | A++ | This invention |
| 917 | G-10 | 0.2 | BA-12 | 3.8 | Hydrophobizing agent (1) | 10 | A++ | This invention |

Note:
¹⁾Addition amount with respect to 100 parts by mass of the cellulose acylate

Meanwhile, the materials newly used in Table 7 are as follows.
IRGANOX (registered trademark) 1010: manufactured by BASF Japan Ltd. Reductone L: 6-O-palmitoyl-L-ascorbic acid (manufactured by Tokyo Chemical Industry Co., Ltd.)
TINUVIN (registered trademark) 123: manufactured by BASF Japan Ltd.
CHELEST PH-540: manufactured by Chelest Corporation
Tekuran DO: manufactured by Nagase ChemteX Corporation
Poem K-37V: manufactured by Riken Vitamin Co., Ltd.
EPOMIN PP-061: manufactured by Nippon Shokubai Co., Ltd.

Meanwhile, in Table 7, IRGANOX (registered trademark) 1010 is described as IRGANOX1010, and TINUVIN (registered trademark) 123 is described as TINUVIN123.

As can be seen from Table 7, it has been found that the durability of polarizer is further improved by the combination use of the compound represented by formula (I) and the compound represented by formula (A), each according to the present invention.

As a result, it is possible to produce a liquid crystal display exhibiting excellent performance as described above, by using the polarizing plate of the present invention.

Having described our invention as related to the present embodiments, it is our intention that the invention not be limited by any of the details of the description, unless otherwise specified, but rather be construed broadly within its spirit and scope as set out in the accompanying claims.

### REFERENCE SIGNS LIST

21A, 21B Polarizing plate
22 Color filter substrate
23 Liquid crystal layer (liquid crystal sell)
24 Array substrate
25 Light-guide plate
26 Light source
31a, 31a′, 31b Optical film (polarizing plate protective film)
311a Cellulose acylate film
311b Hard coat layer
32 Polarizer

R Polarization direction

1. An optical film, comprising:
a cellulose acylate; and
at least one compound represented by formula (I): 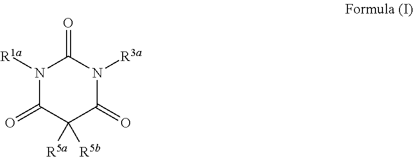
wherein R^{1a} and R^{3a} each independently represent a hydrogen atom, an alkyl group, an alkenyl group, a cycloalkyl group, a cycloalkenyl group, an aryl group, or a heterocyclic group; R^{5a} represents a halogen atom, a cyano group, or a substituent bonded to the barbituric acid skeleton via a heteroatom, —C(═)—, —C(R^{5x})═Y—, or an ethynyl group; X represents an oxygen atom, a sulfur atom, or N(Ra); Ra represents a hydrogen atom, an alkyl group, an aryl group, a heterocyclic group, a hydroxy group, an acyloxy group, an alkoxy group, an aryloxy group, an alkylthio group, an arylthio group, an amino group, an alkylamino group, an arylamino group, or a heterocyclic amino group; R^{5x} represents a hydrogen atom or a substituent; Y represents a nitrogen atom; R^{5b} represents a substituent; and each group of R^{1a}, R^{3a} and R^{5b} may be substituted with a substituent.

2. The optical film according to claim 1, wherein R^{5a} is a halogen atom, or a substituent bonded to the barbituric acid skeleton via a heteroatom or —C(═)—.

3. The optical film according to claim 1, wherein R^{5a} is a substituent bonded to the barbituric acid skeleton via an oxygen atom, a sulfur atom or a nitrogen atom.

4. The optical film according to claim 1, wherein R^{5a} is a hydroxy group, an alkoxy group, an aryloxy group or an acyloxy group.

5. The optical film according to claim 1, wherein R^{5a} is a hydroxy group.

6. The optical film according to claim 1, wherein R^{5b} is an alkyl group, an alkenyl group, a cycloalkyl group, a cycloalkenyl group or an aryl group.

7. The optical film according to claim 1, wherein a sum of ring structures presented in R^{1a}, R^{3a}, R^{5a} and R^{5b} is 2 or more.

8. The optical film according to claim 1, wherein at least one of R^{1a} and R^{3a} is an alkyl group, an alkenyl group, a cycloalkyl group, a cycloalkenyl group, an aryl group, or a heterocyclic group.

9. The optical film according to claim 1, wherein at least one of R^{1a} and R^{3a} is an aryl group, or an alkyl group substituted with an aryl group.

10. The optical film according to claim 1, further comprising a compound represented by formula (A): 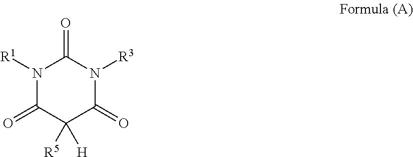
wherein R¹ and R³ each independently represent a hydrogen atom, an alkyl group, an alkenyl group, a cycloalkyl group, a cycloalkenyl group, an aryl group, or a heterocyclic group; R⁵ represents a hydrogen atom or a substituent; and each group of R¹, R³ and R⁵ may be substituted with a substituent.

11. The optical film according to claim 10, wherein a sum of ring structures presented in R¹, R³and R⁵ is 2 or more.

12. The optical film according to claim 10, wherein R¹, R³ and R⁵ each are a group having a ring structure.

13. The optical film according to claim 12, wherein structures of R¹ and R^{1a} and structures of R³ and R^{3a} are respectively the same as each other, in the compound represented by formula (I) and the compound represented by formula (A) contained in the optical film.

14. The optical film according to claim 13, wherein structures of R⁵ and R^{5b} are further the same as each other, in the compound represented by formula (I) and the compound represented by formula (A) contained in the optical film.

15. The optical film according to claim 1, wherein the total acyl substitution degree (A) of the cellulose acylate satisfies the following formula.
1.5≦A≦3.0

16. The optical film according to claim 1, wherein the acyl group of the cellulose acylate is an acetyl group, and the total acetyl substitution degree (B) of the cellulose acylate satisfies the following formula.
2.0≦B≦3.0

17. A polarizing plate, comprising:
a polarizer; and
the optical film according to claim 1, provided on at least one face of the polarizer.

18. A liquid crystal display, at least comprising:
the polarizing plate according to claim 17; and
a liquid crystal cell.

19. A compound represented by formula (II): 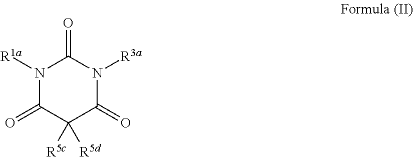
wherein R^{1a} and R^{3a} each independently represent a hydrogen atom, an alkyl group, an alkenyl group, a cycloalkyl group, a cycloalkenyl group, an aryl group, or a heterocyclic group; R^{5c} represents a hydroxy group; R^{5d} represents an alkyl group, an alkenyl group, a cycloalkyl group, a cycloalkenyl group, or an aryl group; each group of R^{1a}, R^{3a} and R^{5d} may be substituted with a substituent; and a sum of ring structures presented in R^{1a}, R^{3a}, and R^{5d} is 2 or more.
